SEQUENCE LISTING (38 sequences)

SEQ ID NO: 1 - protein, 9 aa, Artificial Sequence
  HPV 16E7
RAHYNIVTF

SEQ ID NO: 2 - protein, 9 aa, Artificial Sequence
  HPV Y9T
YMLNLGPET

SEQ ID NO: 3 - protein, 10 aa, Artificial Sequence
  HIV RGP10
RGPGRAFVTI

SEQ ID NO: 4 - protein, 9 aa, Artificial Sequence
  HIV AMQ9
AMQMLKETI

SEQ ID NO: 5 - protein, 64 aa, Artificial Sequence
  RSV Subtype A
MENTSITIEFSSKFWPYFTLIHMITTIISLLIIISIMIAILNKLCEYNVFHNKTFELPRARVNT

SEQ ID NO: 6 - protein, 65 aa, Artificial Sequence
  RSV Subtype B
MGNTSITIEFTSKFWPYFTLIHMILTLISLLIIITIMIAILNKLSEHKTFCNKTLEQGQMYQINT

SEQ ID NO: 7 - protein, 23 aa, Artificial Sequence
  RSV SHeA
NKLCEYNVFHNKTFELPRARVNT

SEQ ID NO: 8 - protein, 24 aa, Artificial Sequence
  RSV SHeB
NKLSEHKTFCNKTLEQGQMYQINT

SEQ ID NO: 9 - protein, 23 aa, Artificial Sequence
  RSV SHeA C45S
NKLSEYNVFHNKTFELPRARVNT

SEQ ID NO: 10 - protein, 40 aa, Artificial Sequence
  RSV bSHeA
NKLCDLNDHHTNSLDIRTRLRNDTQLITRAHEGSINQSSN

SEQ ID NO: 11 - protein, 40 aa, Artificial Sequence
  RSV bSHeA C45S
NKLSDLNDHHTNSLDIRTRLRNDTQLITRAHEGSINQSSN

SEQ ID NO: 12 - protein, 24 aa, Artificial Sequence
  RSV SHeB C51S
NKLSEHKTFSNKTLEQGQMYQINT

SEQ ID NO: 13 - protein, 24 aa, Artificial Sequence
  RSV SHeB C45S
NKLCEHKTFSNKTLEQGQMYQINT

SEQ ID NO: 14 - protein, 29 aa, Artificial Sequence
  RSV L-SHeB C51S
CGGGSNKLSEHKTFSNKTLEQGQMYQINT

SEQ ID NO: 15 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 16 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 17 - protein, 9 aa, Artificial Sequence
  Survivin HLA-A1
FEELTLGEF

SEQ ID NO: 18 - protein, 9 aa, Artificial Sequence
  Survivin HLA-A1 (modified)
FTELTLGEF

SEQ ID NO: 19 - protein, 9 aa, Artificial Sequence
  Survivin HLA-A2
LTLGEFLKL

SEQ ID NO: 20 - protein, 9 aa, Artificial Sequence
  Survivin HLA-A2 (modified)
LMLGEFLKL

SEQ ID NO: 21 - protein, 10 aa, Artificial Sequence
  Survivin HLA-A3
RISTFKNWPF

SEQ ID NO: 22 - protein, 10 aa, Artificial Sequence
  Survivin HLA-A3 (modified)
RISTFKNWPK

SEQ ID NO: 23 - protein, 9 aa, Artificial Sequence
  Survivin HLA-A24
STFKNWPFL

SEQ ID NO: 24 - protein, 9 aa, Artificial Sequence
  Survivin HLA-B7
LPPAWQPFL

SEQ ID NO: 25 - protein, 27 aa, Artificial Sequence
  Mut25
STANYNTSHLNNDVWQIFENPVDWKEK

SEQ ID NO: 26 - protein, 27 aa, Artificial Sequence
  Mut30
PSKPSFQEFVDWENVSPELNSTDQPFL

SEQ ID NO: 27 - protein, 27 aa, Artificial Sequence
  Mut44
EFKHIKAFDRTFANNPGPMVVFATPGM

SEQ ID NO: 28 - protein, 16 aa, Artificial Sequence
  A16Y T-helper epitope
AQYIKANSKFIGITEL

SEQ ID NO: 29 - protein, 13 aa, Artificial Sequence
  PADRE T-helper epitope
  MISC_FEATURE (3)..(3): Xaa may be cyclohexylalanyl
AKXVAAWTLKAAA

SEQ ID NO: 30 - protein, 21 aa, Artificial Sequence
  F21E T-helper epitope
FNNFTVSFWLRVPKVSASHLE

SEQ ID NO: 31 - DNA, 20 bp, Artificial Sequence
  CpG oligonucleotide
tccatgacgttcctgacgtt

SEQ ID NO: 32 - DNA, 26 bp, Artificial Sequence
  PolyIC oligonucleotide (dIdC)
  modified_base (1)..(1): Inosine
  misc_feature (1)..(1): n is a, c, g, or t
  modified_base (3)..(3): Inosine
  misc_feature (3)..(3): n is a, c, g, or t
  modified_base (5)..(5): Inosine
  misc_feature (5)..(5)
  misc_feature (5)..(5): n is a, c, g, or t
  modified_base (7)..(7): Inosine
  misc_feature (7)..(7)
  misc_feature (7)..(7): n is a, c, g, or t
  modified_base (9)..(9): Inosine
  misc_feature (9)..(9)
  misc_feature (9)..(9): n is a, c, g, or t
  modified_base (11)..(11): Inosine
  misc_feature (11)..(11): n is a, c, g, or t
  modified_base (13)..(13): Inosine
  misc_feature (13)..(13): n is a, c, g, or t
  modified_base (15)..(15): Inosine
  misc_feature (15)..(15): n is a, c, g, or t
  modified_base (17)..(17): Inosine
  misc_feature (17)..(17): n is a, c, g, or t
  modified_base (19)..(19): Inosine
  misc_feature (19)..(19): n is a, c, g, or t
  modified_base (21)..(21): Inosine
  misc_feature (21)..(21): n is a, c, g, or t
  modified_base (23)..(23): Inosine
  misc_feature (23)..(23): n is a, c, g, or t
  modified_base (25)..(25): Inosine
  misc_feature (25)..(25): n is a, c, g, or t
ncncncncncncncncncncncncnc

SEQ ID NO: 33 - protein, 6 aa, Artificial Sequence
  Palmitic Acid Adjuvant
CSKKKK

SEQ ID NO: 34 - protein, 22 aa, Artificial Sequence
  Fusion Peptide PADRE and R9F
  MISC_FEATURE (3)..(3): Xaa may be cyclohexylalanyl
AKXVAAWTLKAAARAHYNIVTF

SEQ ID NO: 35 - protein, 9 aa, Artificial Sequence
  MAGE-A9 111
KVAELVHFL

SEQ ID NO: 36 - protein, 9 aa, Artificial Sequence
  MAGE-A9 24
GLMGAQEPT

SEQ ID NO: 37 - protein, 9 aa, Artificial Sequence
  MAGE-A9 223
ALSVMGVYV

SEQ ID NO: 38 - protein, 9 aa, Artificial Sequence
  MAGE-A9 270
FLWGSKAHA

(19) United States
(12) Patent Application Publication
(10) Pub. No.: US 2023/0000769 A1
(43) Pub. Date: Jan. 5, 2023

(54) OIL-IN-WATER EMULSION FORMULATIONS FOR DELIVERY OF ACTIVE OR THERAPEUTIC AGENTS

(71) Applicant: IMMUNOVACCINE TECHNOLOGIES INC., Dartmouth (CA)

(72) Inventors: Rajkannan RAJAGOPALAN, Dartmouth (CA); Marianne STANFORD, Upper Tantallon (CA); Heather TORREY, Halifax (CA)

(73) Assignee: IMMUNOVACCINE TECHNOLOGIES INC., Dartmouth (CA)

(21) Appl. No.: 17/768,335

(22) PCT Filed: Oct. 15, 2020
(86) PCT No.: PCT/CA2020/051377

§ 371 Date: Apr. 12, 2022

Related U.S. Application Data
(60) Provisional application No. 62/915,696, filed on Oct. 16, 2019.

(51) Int. Cl. A61K 9/107; A61K 47/44; A61K 47/26; A61K 47/24

(52) U.S. Cl. CPC A61K 9/107; A61K 47/44; A61K 47/26; A61K 47/24

(57) ABSTRACT

Provided herein are methods and compositions for the delivery of at least two active, therapeutic, or pharmaceutical agents in an oil-in-water emulsion, wherein at least one agent is delivered in the hydrophobic phase of the emulsion and at least one agent is delivered in the aqueous phase of the emulsion.

### CROSS-REFERENCE TO RELATED APPLICATIONS

This application claims the benefit of and priority from U.S. Provisional Patent Application No. 62/915,696, filed Oct. 16, 2019.

### FIELD

The present invention relates to methods and compositions for the delivery of at least two active, therapeutic, or pharmaceutical agents in an oil-in-water emulsion, wherein at least one agent is delivered in the hydrophobic phase of the emulsion and at least one agent is delivered in the aqueous phase of the emulsion.

### BACKGROUND

In the pharmaceutical field, the effective delivery of active, therapeutic, and pharmaceutical agents often poses difficulties and challenges. Although a particular agent may be effective in vitro, the in vivo effectiveness of an agent in a subject will further depend on the method of delivery, route of administration, and pharmacokinetics in the body. For example, an agent's in vivo effectiveness may depend on its ability to be targeted to a specific tissue or cell type, or instead on its ability to be delivered systemically throughout the body. Furthermore, an agent's effectiveness in vivo may also depend on its rate of release from the site of administration: either a sustained release or an immediate release. The considerations will depend on the nature of a particular agent (e.g. size, stability, solubility, charge) and its desired effects and/or targets.

The targeting of an agent is influenced by the route of administration. An agent that needs to be delivered systemically can be injected intravenously for immediate circulation through the blood or delivered orally for absorption into the blood through the digestive system. Conversely, an agent can be targeted to a particular tissue by injection directly into a specific organ or tissue, or by injection into a site that drains into a specific organ or tissue. For example, an agent can be injected sub-cutaneously for targeting of the agent into a draining lymph node. Alternatively, an agent can be modified by linking it to a targeting molecule and then delivering the agent systemically, with the agent then accumulating in the target tissue or on target cells via the targeting molecule. However, this requires chemical modification of the agent, possibly changing its properties, and adds cost and complexity to the therapy.

The rate of release of an agent can be controlled by its formulation in a pharmaceutical composition. For example, an agent may be provided in an oral tablet with a chemical coating that ensures a sustained release of the agent during digestion. An injected agent may be formulated in an aqueous solution that disperses quickly after injection by dissolving into interstitial fluid or blood, or draining into the lymphatic system, thereby providing an immediate release of the agent. Conversely, an injected agent may be delivered in an oil-based composition that provides a depot-effect, thereby providing a sustained release of the agent.

Often, a therapy for a disease, disorder, or infection requires the administration of more than one agent. However, challenges arise when the agents have different properties and/or desired targets. For example, it may be desired than a first agent in a therapy has a sustained release while a second agent has a faster or immediate release. In another example, it may be desired that a first agent in a therapy is targeted to a specific tissue, such as a lymphatic tissue, while a second agent is released systemically. A common solution is to provide the agents separately. This approach holds several disadvantages. The administration of multiple agents by different methods and with different regimens complicates the therapeutic protocol and may lead to mistakes in administration. Furthermore, if the approach requires multiple injections to separately deliver the agents there is an increase in patient discomfort and possibly increased risk of unwanted injection-site reactions.

There is therefore a need for new and effective means of delivering multiple agents to a subject in a single composition or administration that accommodates the differing properties of the multiple agents and their desired targets and rates of release. Such a composition or administration could simplify therapies that require multiple agents and improve patient comfort. Furthermore, such a composition or administration could also improve the effectiveness of the therapy by improving the efficacy of the agents and, therefore, the treatment of the subject.

Accordingly, there is provided herein methods and compositions for delivering multiple agents in separate phases of an oil-in-water emulsion to a subject. The methods and compositions of the present invention are able to co-deliver aqueous phase agents and hydrophobic phase agents, improve the efficacy of the delivered agents, and generate lower titers of unwanted anti-drug antibody in a subject. As demonstrated in Examples 7 and 8, treatment of tumour-challenged mice with an emulsion composition according to the present invention (comprising a DPX anti-cancer composition in the hydrophobic phase and immunomodulatory anti-CTLA-4 antibody in the aqueous phase) improved survival and tumour control and generated lower titers of unwanted anti-drug antibody (ADA) against anti-CTLA4 antibody compared to other compositions.

### SUMMARY

In an embodiment, the present disclosure relates to a composition for delivering at least two agents to a subject comprising: i) a hydrophobic phase; and ii) an aqueous phase; wherein the composition is an emulsion of the hydrophobic phase in the aqueous phase, wherein the hydrophobic phase comprises at least one hydrophobic phase agent, and wherein the aqueous phase comprises at least one aqueous phase agent.

In an embodiment, the present disclosure relates to a composition for delivering at least two agents to a subject comprising: i) a hydrophobic phase comprising mannide oleate in mineral oil, DOPC, cholesterol, peptide antigen of SEQ ID NO: 1, T-helper epitope of SEQ ID NO: 30, and DNA based polyL:C; and ii) an aqueous phase comprising water and/or an aqueous solution, polysorbate 20, and an antibody that binds to CTLA-4; wherein the composition is an emulsion of the hydrophobic phase in the aqueous phase.

In an embodiment, the present disclosure relates to a composition for delivering at least two agents to a subject comprising: i) a hydrophobic phase comprising mannide oleate in mineral oil, DOPC, cholesterol, peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C; and ii) an aqueous phase comprising water and/or an aqueous solution, polysorbate 20, and an antibody that binds to CTLA-4; wherein the composition is an emulsion of the hydrophobic phase in the aqueous phase.

In an embodiment, the present disclosure relates to a composition for delivering at least two agents to a subject comprising: i) a hydrophobic phase comprising mannide oleate in mineral oil, DOPC, cholesterol, fusion peptide of SEQ ID NO: 34, and DNA based polyL:C; and ii) an aqueous phase comprising water and/or an aqueous solution, polysorbate 20, and an antibody that binds to CTLA-4; wherein the composition is an emulsion of the hydrophobic phase in the aqueous phase.

In an embodiment, the present disclosure relates to a composition for delivering at least two agents to a subject comprising: i) a hydrophobic phase comprising mannide oleate in mineral oil, DOPC, cholesterol, peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C; and ii) an aqueous phase comprising water and/or an aqueous solution, polysorbate 20, and an antibody that binds to CTLA-4; wherein the composition is an emulsion of the hydrophobic phase in the aqueous phase.

In an embodiment, the present disclosure relates to a method for making a composition for delivering at least two agents to a subject, said method comprising: i) providing a hydrophobic phase comprising at least one hydrophobic phase agent; ii) providing an aqueous phase comprising at least one aqueous phase agent; iii) mixing the hydrophobic phase and the aqueous phase to produce an emulsion of the hydrophobic phase in the aqueous phase. In an embodiment, the present disclosure relates to a composition produced by the methods described herein.

In an embodiment, the present disclosure relates to a method for delivering at least two agents to a subject, said method comprising administering to the subject a composition as described herein.

In an embodiment, the present disclosure relates to a method for inducing an immune response in a subject, comprising administering to the subject a composition as described herein.

In an embodiment, the present disclosure relates to a method for treating, preventing or diagnosing a disease, disorder or condition in a subject, comprising administering to the subject a composition as described herein.

In an embodiment, the present disclosure relates to a method for modulating an immune response in a subject, comprising administering to the subject a composition as described herein.

In an embodiment, the present disclosure relates to a method for treating or preventing diseases and/or disorders ameliorated by a cell-mediated immune response or a humoral immune response in a subject, comprising administering to the subject a composition as described herein.

In an embodiment, the present disclosure relates to a method for treating and/or preventing an infectious disease caused by a virus, bacteria, or protozoa in a subject, comprising administering to the subject a composition as described herein.

In an embodiment, the present disclosure relates to a method for treating and/or preventing cancer in a subject, comprising administering to the subject a composition as described herein.

In an embodiment, the present disclosure relates to a method for neutralizing a toxin, virus, bacterium, or allergen with an antibody in a subject, said method comprising administering to the subject a composition as described herein.

In an embodiment, the present disclosure relates to a kit comprising: a) a first container comprising a dried preparation of at least one hydrophobic phase agent; b) a second container comprising one or more hydrophobic substances; and c) a third container comprising an aqueous solution comprising at least one aqueous phase agent.

In an embodiment, the present disclosure relates to a kit comprising: a) a first container comprising a dried preparation of at least one hydrophobic phase agent; b) a second container comprising one or more hydrophobic substances; c) a third container comprising a dried preparation of at least one aqueous phase agent; and d) a fourth container comprising water, an aqueous solution, or a combination thereof.

Other aspects and features of the present invention will become apparent to those of ordinary skill in the art upon review of the following description in conjunction with the accompanying figures.

### BRIEF DESCRIPTION OF THE FIGURES

The accompanying figures, which constitute a part of this specification, illustrate embodiments of the invention by way of example only.

FIG. 1 shows a study timeline in days and the study days on which treatments were administered or samples taken.

FIG. 2 shows the percent survival of mice from the treatment groups 1-9 (A) and groups 5-8 (B) over time. mCPA=metronomic cyclophosphamide (administered orally); IP=intraperitoneal (injection). Survival statistical analysis was performed using the Mantel-Cox test, ***p<0.001, *p<0.05.

FIG. 3 shows the tumour volume in mice from the treatment groups 1-9 (A) and groups 5-8 (B) over time. mCPA=metronomic cyclophosphamide (administered orally); IP=intraperitoneal (injection). Tumour volume statistical analysis was performed by linear regression comparison, ***p<0.0001.

FIG. 4 shows a schematic representation of an exemplary oil-in-water emulsion formulation. The hydrophobic phase comprising a hydrophobic phase agent (Syringe 1) is mixed with the aqueous phase comprising an aqueous phase agent (Syringe 2) using a connector to form an O/W emulsion.

FIG. 5 shows the titers of anti-drug antibody (ADA) against anti-CTLA4 antibody in mice treated with various compositions. ADA formation was detected by bridging ELISA with anti-CTLA-4 coating and detection antibody (A), IgG2b isotype control coating antibody and anti-CLA-4 detection antibody (B), and IgG1 isotype control coating antibody and anti-CTLA-4 detection antibody (C). Statistical significance was assessed by one-way ANOVA using Tukey's multiple comparisons test, *p<0.05.

FIG. 6 shows HPLC chromatograms of an exemplary oil-in-water emulsion formulation with a hydrophobic phase comprising DPX-empty in oil and an aqueous phase comprising an oligonucleotide aqueous phase agent. (A) chromatogram of an oligonucleotide standard, (B) chromatogram of the hydrophobic phase (top layer-oil), (C) chromatogram of the aqueous phase (bottom layer-water).

FIG. 7 shows HPLC chromatograms of an exemplary oil-in-water emulsion formulation with a hydrophobic phase comprising DPX-empty in oil and an aqueous phase comprising a cyclophosphamide aqueous phase agent. (A) chromatogram of a cyclophosphamide standard, (B) chromatogram of the aqueous phase (bottom layer-water).

FIG. 8 shows HPLC chromatograms of an exemplary oil-in-water emulsion formulation with a hydrophobic phase comprising DPX-empty in oil and an aqueous phase comprising an anti-CTLA4 antibody aqueous phase agent. (A) chromatogram of an anti-CTLA4 standard, (B) chromatogram of the hydrophobic phase (top layer-oil), (C) chromatogram of the aqueous phase (bottom layer-water).

### DETAILED DESCRIPTION

The present invention relates to methods and compositions for the delivery of at least two active, therapeutic, and/or pharmaceutical agents in an emulsion of a hydrophobic phase in an aqueous phase, wherein at least one agent is delivered in the hydrophobic phase of the emulsion (a hydrophobic phase agent) and at least one agent is delivered in the aqueous phase of the emulsion (an aqueous phase agent). The emulsion comprises a hydrophobic phase that provides the sustained release of at least one hydrophobic phase agent and provides targeted delivery to immune cells, lymph nodes, or lymphoid cells in a lymphatic tissue. The emulsion further comprises an aqueous phase of that provides the faster release of at least one aqueous phase agent, compared to the rate of release of the at least one hydrophobic phase agent, and a wider dispersal from the site of administration.

In order to provide a therapy for treating a disease, disorder, or infection in a subject, it may be necessary to provide more than one active, therapeutic, or pharmaceutical agent. In such a situation, it may be advantageous to provide the more than one agent in the same composition, for a single administration to the subject. The agents may have different desired rates of release and/or desired in vivo targets to one another. The present invention therefore provides methods and compositions for the delivery of at least two agents in an oil-in-water emulsion, consisting of a hydrophobic phase dispersed in an aqueous phase. Agents to be delivered to the subject can be incorporated into either the hydrophobic phase for a sustained release and targeted delivery, or into the aqueous phase for a faster release and wider dispersal within the subject, while also being incorporated into a single composition that can be provided in a single administration.

The emulsion comprises a hydrophobic phase that provides a sustained release of at least one hydrophobic phase agent, with the at least one hydrophobic phase agent targeted to immune cells, lymph nodes, or lymphoid cells in a lymphatic tissue.

As used herein, by “sustained release”, it is meant that the agent is available for a prolonged period of time for uptake by immune cells from the site of administration. In an embodiment, the term “sustained release” means that a substantial proportion (e.g. at least 50%, at least 60%, at least 70%, at least 80%, at least 90%, at least 95%, or 100%) of the agent remains localized at the site of administration for at least about 36 hours, 48 hours, 72 hours, 96 hours, 120 hours, 144 hours, 168 hours, 192 hours, or 384 hours. In an embodiment, less than 1%, 2%, 3%, 4% or 5% of the agent is taken by immune cells from the site of administration within 24 hours after administration. In an embodiment, at least 80% of the agent remains localized at the site of injection for at least about 48 hours after administration. In an embodiment, at least 60% of the agent remains localized at the site of administration for at least about 72 hours after administration.

As used herein, by “targeted” or “targeting”, it is meant that the at least one hydrophobic phase agent is preferentially delivered to immune cells, lymph nodes, or lymphoid cells in a lymphatic tissue. As used herein, “preferentially delivered” refers to the fact that the at least one hydrophobic phase agent is delivered to immune cells, lymph nodes, or lymphoid cells in a lymphatic tissue as opposed to being delivered to other regions of the body or being delivered systemically. In an embodiment, by “preferentially delivered”, it is meant that the concentration or amount of the at least one hydrophobic phase agent is increased in the immune cells, lymph nodes, or lymphoid cells in a lymphatic tissue relative to the concentration or amount of the at least one hydrophobic phase agent in other parts of the body.

As used herein, and without being bound by theory, the term “targeted delivery” encompasses embodiments whereby the targeting to immune cells, lymph nodes, or lymphoid cells in a lymphatic tissue is accomplished by upstream events whereby the at least one hydrophobic phase agent is more effectively taken up by immune cells (such as by phagocytosis or endocytosis) or delivered to antigen presenting cells that are capable of trafficking the at least one hydrophobic phase agent to lymph nodes or lymphoid cells in a lymphatic tissue. In an embodiment, the hydrophobic phase agent is taken up by immune cells, such as for example and without limitation, monocytes, macrophages, dendritic cells, T cells and/or B cells. In an embodiment, the hydrophobic phase agent is delivered to antigen presenting cells such as for example and without limitation, monocytes, macrophages, dendritic cells, and/or B cells, and the antigen presenting cells traffic the at least one hydrophobic phase agent to lymph nodes, or lymphoid cells in a lymphatic tissue. Thus, in an embodiment, “targeted delivery to lymph nodes or lymphoid cells in a lymphatic tissue” includes preferential delivery of the at least one hydrophobic phase agent to cells in a non-lymphatic fluid or tissue in the body whereby the cells then traffic the at least one hydrophobic phase agent to lymph nodes or lymphoid cells in a lymphatic tissue.

As used herein, “lymph nodes” refers to any one or more lymph nodes that are present throughout the body of an animal, such as for example a human. In an embodiment, the lymph nodes are any one or more of the following types, based on anatomical location: inguinal (groin), femoral (upper inner thigh), mesentery (lower body below rib cage), mediastinal (upper body behind the sternum); supraclavicular (collar bone); axillary (armpits); and cervical (neck). The lymph node to which the at least one hydrophobic phase agent preferentially targets may depend on the route of administration (e.g. injection) and location of administration. In an embodiment, the lymph nodes are the lymph nodes draining the injection site.

As used herein, the term “lymphatic tissue” refers to the cells and organs that make up the lymphatic system. It includes, without limitation, the lymph nodes, spleen, thymus and mucosal-associated lymphoid tissue (e.g., in the lung, lamina propria of the of the intestinal wall, Peyer's patches of the small intestine, or lingual, palatine and pharyngeal tonsils, or Waldeyer's neck ring). The lymphoid cells of the lymphatic tissue include, for example, leukocytes (white blood cells), T cells (T-lymphocytes), B cells (B-lymphocytes), macrophages, dendritic cells and reticular cells. In an embodiment, the targeted delivery of the at least one hydrophobic phase agent disclosed herein is to T-lymphocytes and/or B-lymphocytes in the lymph nodes or lymphatic tissue.

Without being bound by theory, it is believed that the hydrophobic phase of the composition according to the invention provides for targeted delivery of the at least one hydrophobic phase agent to immune cells, lymph nodes, or lymphoid cells in a lymphatic tissues by one or more of: (i) promoting effective uptake of the at least one hydrophobic phase agent by immune cells (e.g. monocytes, macrophages, dendritic cells, T cells and/or B cells) at or near the site of administration due to the separation of the emulsion at the site of administration, leaving the hydrophobic phase at the site of administration that attracts immune cells and provides extended exposure to the at least one hydrophobic phase agent; (ii) promoting migration of such immune cells to lymph nodes; and (iii) promoting uptake of the at least one hydrophobic phase agent by cells in the lymph nodes or lymphoid cells in a lymphatic tissue.

In some embodiments, the at least one hydrophobic phase agent comprises antigens and/or adjuvants for eliciting an immune response. In some embodiments, the hydrophobic phase is a composition comprising antigens and/or adjuvants for eliciting an immune response.

In embodiments in which the hydrophobic phase comprises antigens and/or adjuvants for eliciting an immune response, targeting of the hydrophobic phase agent to immune cells, lymph nodes, or lymphoid cells in a lymphatic tissue allows for the activation of immune cells in order to elicit the immune response. Prior to encountering foreign antigen, immune cells (e.g. monocytes, macrophages, dendritic cells, T cells and/or B cells) exist in an immature state. Upon phagocytosis of a presentable antigen, antigen-presenting immune cells (e.g. monocytes, macrophages, B cells, and dendritic cells) become activated resulting in an upregulated expression of MHC class I/II molecules and maturation into mature antigen-presenting cells that migrate to the lymph nodes where they interact with lymphocytes (e.g. T cells and B cells) by receptor-mediated interactions. This leads to the activation of the lymphocytes themselves and the induction of an adaptive immune response. In the case of immunotherapy, appropriate activation of immune cells typically also requires administration of an adjuvant to improve routing and adaptive immune responses.

In some embodiments, the at least one hydrophobic phase agent comprises agents that are not antigens and/or adjuvants, but rather other agents (e.g. small molecule drugs, antibodies, immunomodulatory agents, allergens, or polynucleotides) that are targeted to lymph nodes or lymphoid cells in a lymphatic tissue. In some embodiments, the hydrophobic phase is a pharmaceutical composition comprising agents for modulating an immune response. In some embodiments, the hydrophobic phase is a pharmaceutical composition comprising an antibody.

In embodiments in which the hydrophobic phase comprises agents for the purpose of modulating an immune response, targeting of the hydrophobic phase agent to immune cells, lymph nodes, or lymphoid cells in a lymphatic tissue allows for the modulation of immune cells and/or an immune response. Even in the absence of presentable antigen and activation of immune cells, agents provided in a hydrophobic phase can be taken up by immune cells and/or trafficked to lymph nodes or lymphoid cells in a lymphatic tissue for targeted delivery, as disclosed in e.g. PCT/CA2019/050328.

The emulsion comprises an aqueous phase that provides a faster release of at least one aqueous phase agent, compared to the rate of release of at least one hydrophobic phase agent, and a wider dispersal from the site of administration. As used herein, by “wider dispersal”, it is meant that the aqueous phase and the at least one aqueous phase agent disperse from the site of administration rather than form a deposit at the site of administration that does not significantly disperse. For example, the aqueous phase and/or the at least one aqueous phase agent may dissolve into the surrounding interstitial fluid and spread throughout a tissue or organ. In another example, the aqueous phase and/or the at least one aqueous phase agent may dissolve into lymphatic fluid or blood and enter circulation to provide systemic delivery. As used herein, by “systemic delivery”, it is meant that the at least one aqueous phase agent is delivered throughout the body so that multiple tissues, multiple organs, or the entire body are exposed to therapeutically effective amounts of the agent. Agents that are delivered systemically typically enter the circulatory system, either directly or indirectly, where they are circulated throughout the body via the bloodstream. As used herein, “systemic delivery” encompasses embodiments wherein the at least one aqueous phase agent disperses from the site of administration and enters circulation, either directly or indirectly, to provide a therapeutically effective amount of the agent to multiple tissues, multiple organs, or the entire body. The aqueous phase provides for the faster release of at least one aqueous phase agent as compared to the rate of release of at least one hydrophobic phase agent. In some embodiments, an emulsion of the present invention provides a slower release of at least one aqueous phase agent compared to a conventional aqueous formulation.

Without being bound by theory, it is believed that the aqueous phase of the composition according to the invention provides for the faster release of at least one aqueous phase agent, compared to the rate of release of at least one hydrophobic phase agent, and the wider dispersal from the site of administration by dissolving into the interstitial fluid, allowing the at least one aqueous phase agent to dissolve into the interstitial fluid whereby it can: (i) disperse into the surrounding tissue or organ; (ii) diffuse into circulation through capillary walls; (iii) and/or enter lymphatic circulation and, subsequently, enter the bloodstream through lymphatic vessels.

The methods and compositions of the present invention are advantageous in providing a single composition for the delivery of at least two agents with differing targets, properties and release rates. The present invention can be used for the delivery of at least 2 agents to a subject when it is necessary that at least one agent be targeted to immune cells, lymph nodes, or lymphoid cells in a lymphatic tissue in a sustained release while at least one other agent be dispersed more widely and rapidly in the subject. By way of non-limiting example, an emulsion composition of the present invention can provide an antigen in the hydrophobic phase and an immunomodulatory agent in the aqueous phase in order to induce an improved immune response in a subject. In this manner, the emulsion composition provides a sustained, targeted release of the antigen to immune cells while simultaneously providing a faster release of an immunomodulatory agent to improve the immune response to the antigen. Furthermore, methods and compositions of the present invention can also improve the efficacy of the delivered agents. As demonstrated in Example 7, treatment of tumour-challenged mice with an emulsion composition according to the present invention (comprising a DPX anti-cancer composition in the hydrophobic phase and immunomodulatory anti-CTLA-4 antibody in the aqueous phase) improved survival and tumour control compared to mice receiving the DPX anti-cancer composition and anti-CTLA-4 antibody either separately, or together in a composition lacking an O/W emulsion. A further advantage of the present invention is demonstrated in Example 8, showing that an emulsion composition according to the present invention generated lower titers of unwanted anti-drug antibody (ADA) against anti-CTLA4 antibody compared to different compositions lacking the O/W emulsion of the present invention.

Emulsions

As used herein, an “emulsion” refers to a mixture of two or more liquids that are normally immiscible wherein droplets of one liquid are dispersed in the other. As an example, a hydrophobic substance (e.g. oil) and an aqueous substance (e.g. water) are immiscible liquids that may form an emulsion when droplets of one are dispersed in the other. A dispersion of water droplets in oil is a water-in-oil (W/O) emulsion in which the water (aqueous phase) forms a discontinuous phase and the oil (hydrophobic phase) forms a continuous phase. “Water-in-oil emulsion” or “W/O”, as used herein, refers to an emulsion of a hydrophobic phase in an aqueous phase. A dispersion of oil droplets in water is an oil-in-water (O/W) emulsion in which the oil (hydrophobic phase) forms a discontinuous phase and the water (aqueous phase) forms a continuous phase. “Oil-in-water emulsion” or “O/W”, as used herein, refers to an emulsion of a hydrophobic phase in an aqueous phase. A phase or substance that is hydrophobic may also be called lipophilic. A phase or substance that is aqueous may also be called hydrophilic or lipophobic.

An emulsion may be described as stable if the discontinuous phase remains dispersed in the continuous phase for a prolonged period of time. As described herein, an emulsion may be described as stable if the discontinuous phase remains dispersed in the continuous phase for 1 hour, 2 hours, 3 hours, 4 hours, or greater than 4 hours after the formation of the emulsion. The phases of an emulsion may separate over time. Phase separation may be due to the buoyancy of the dispersed phase, causing the droplets to sink or float in the continuous phase; due to the coalescence of the droplets into gradually larger droplets; or due to the flocculation of mutually-attracted droplets. Phase separation of an emulsion can be determined visually by seeing if the emulsion appears homogenous or if there is a detectable separation of one phase from the other.

In some embodiments, emulsifiers may be included in one or more of the phases of the emulsion. As used herein, an “emulsifier” refers to a substance or compound that enables the formation of an emulsion and/or improves the stability of the emulsion. An emulsifier may be, for example, a lipid, a surfactant, detergent, or emulsifying salt. Emulsifiers may enable to formation of an emulsion and/or improve the stability of an emulsion by dispersing and/or stabilizing the droplets of the discontinuous phase, thereby preventing phase separation. Emulsifiers may be amphiphilic, possessing both a polar or hydrophilic region and a non-polar or hydrophobic (i.e. lipophilic) region enabling them to interact with both hydrophobic and aqueous phases. The affinity of an emulsifier for water or oil is measured by its hydrophile-lipophile balance (HLB). HLB is a 0-20 scale: HLB values below 10 indicate a greater affinity for oil and for forming W/O emulsions; HLB values above 10 indicate a greater affinity for water and for forming O/W emulsions. Emulsifiers may further be classified as ionic or non-ionic depending on the presence of an ionic group. The choice of emulsifier(s) for use in stabilizing an emulsion will depend on the desired properties of the emulsion such as O/W vs W/O, density, viscosity, dispersion rate in water or oil, and stability. Emulsifiers that may be used to formulate an O/W emulsion according to the present invention include, but are not limited to polysorbate 20 (e.g. Tween™ 20), polysorbate 40 (e.g. Tween™ 40), polysorbate 60 (e.g. Tween™ 60), polysorbate 80 (e.g. Tween™ 80), lecithin, mannide oleate, sorbitan monolaurate (e.g. Span™ 20), sorbitan tristearate (e.g. Span™ 65), sorbitan monooleate (e.g. Span™ 80), sorbitan trioleate (e.g. Span™ 85), Nonoxynols Triton™ X-100, Octaethylene glycol monododecyl ether, Pentaethylene glycol monododecyl ether, Poloxamers, Glycerol monostearate, Glycerol monolaurate, Decyl glucoside, Lauryl glucoside, Octyl glucoside, Lauryldimethylamine oxide, Dimethyl sulfoxide, Phosphine oxide, Polyethoxylated tallow amine, Cocamide monoethanolamine, Cocamide diethanolamine MONTANE™ 20, 80 and 85 PPI emulsifiers and MONTANOX™ 20, 80 PPI and MONTANOX™ 80 API solubilizers, Anionic surfactants such as ammonium lauryl sulfate, sodium lauryl sulfate, sodium dodecyl sulfate, sodium lauryl ether sulfate, sodium myreth sulfate, dioctyl sodium sulfosuccinate, Perfluorooctanesulfonate, Perfluorobutanesulfonate, Alkyl-aryl ether phosphates and Alkyl ether phosphates, Carboxylates surfactants such as sodium stearate, sodium lauroyl sarcosinate, perfluorononanoate and perfluorooctanoate, Cationic Surfactants such as octenidine dihydrochloride, cetrimonium bromide, cetylpyridinium chloride, benzalkonium chloride, benzethonium chloride, dimethyldioctadecylammonium chloride, and dioctadecyldimethylammonium bromide, Zwitterionic surfactants such as lauryl-N,N-(dimethylammonio)butyrate, lauryl-N,N-(dimethyl)-glycinebetaine, Cocamidopropyl betaine, 3-[(3-cholamidopropyl)dimethylammonio]-1-propanesulfonate, 3-([3-cholamidopropyl]dimethylammonio)-2-hydroxy-1-propanesulfonate, 3-[(3-cholamidopropyl)dimethylammo-nio]-1-propanesulfonate, lauryl-N,N-(dimethylammonio)butyrate, lauryl-N,N-(dimethyl)-propanesulfonate, 3-(4-tert-butyl-1-pyridinio)-1-propanesulfonate, 3-(1-pyridinio)-1-propanesulfonate, 3-(benzyl-dimethylammonio)propanesulfonate and Dipalmitoylphosphatidylcholine.

Compositions according to the present invention comprise an O/W emulsion of a hydrophobic phase (e.g. oil) in an aqueous phase (e.g. water). Emulsions according to the present invention may be formed using a range of hydrophobic phase to aqueous phase ratios, defined as a volume to volume ratio (v/v). In some embodiments, the hydrophobic phase to aqueous phase ratio may be 90:10, 80:20, 70:30: 60:40, 50:50, 40:60, 30:70, 20:80, or 10:90. The necessary ratio to form an O/W emulsion will depend on the respective compositions of the hydrophobic and aqueous phases (e.g. the presence of amphiphilic compounds) as well as the presence or absence of emulsifiers. A skilled person may determine a suitable ratio by emulsifying the desired hydrophobic and aqueous phases using techniques disclosed herein and then determining whether the emulsion is O/W by performing a water drop test or an oil drop test as disclosed herein. Within the range of ratios capable of forming an O/W emulsion, the ratio may further be adjusted to achieve desired properties such as viscosity, density, and dispersion rate in water.

An emulsion according to the present invention may be formed by a number of techniques known in the art. For example, an emulsion may be formed by mixing the aqueous and hydrophobic phases in a vessel and then agitating the vessel to disperse the hydrophobic phase as droplets in the aqueous phase. The vessel can be agitated by any physical means such as, for example, vortex mixing with a vortex mixer. Alternatively, an emulsion may be formed by repeatedly passing the phases through an aperture. For example, the hydrophobic phase may be placed in one vessel, the aqueous phase may be placed in another vessel, the two vessels are connected via a connector with an aperture, and pressure is applied to pass the phases back and forth between the vessels through the aperture. In a more specific example, the hydrophobic phase is placed in a first syringe, the aqueous phase is placed in a second syringe, the two syringes are connected with a connector, and alternating pressure is applied to the syringes to repeatedly pass the phases through the connector. Syringes should be selected based on the desired volume of the emulsion (e.g. syringes with volumes of 0.5 mL, 1 mL, 2 mL, 5 mL, or more) and their ability to attach to connectors (e.g. Luer Lock syringes or threaded syringes). Suitable connectors for connecting syringes include, but are not limited to, Luer-to-Luer connectors, Luer-to-threaded connectors, threaded connectors, 3-way stopcocks, and Vygon™ connectors/adaptors.

Hydrophobic Phases

An O/W emulsion according to the present invention comprises a discontinuous hydrophobic phase. The hydrophobic phase is immiscible in an aqueous phase. The hydrophobic phase forms an emulsion in an aqueous phase by forming a dispersion of droplets in the aqueous phase. The hydrophobic phase may be dispersed in the aqueous phase to form an emulsion using the techniques disclosed herein. As used herein, a “hydrophobic phase” refers to a mixture comprising one or more hydrophobic substances and at least one agent (a hydrophobic phase agent). A hydrophobic phase may further comprise other ingredients including, but not limited to, lipids, cholesterol, polymers, glycosides, cellulose, buffer salts, cryoprotectants, surfactants, and emulsifiers as described herein.

Hydrophobic Substances

The hydrophobic phase may comprise an essentially pure hydrophobic substance or a mixture of hydrophobic substances. Hydrophobic substances that are useful in the hydrophobic phase are those that are pharmaceutically acceptable. The hydrophobic substances are typically a liquid at room temperature (e.g. about 18-25° C.), but certain hydrophobic substances that are not liquids at room temperature may be liquefied, for example by warming, and may also be useful.

Oil or a mixture of oils is a particularly suitable hydrophobic substance for use in forming the hydrophobic phase. Oils should be pharmaceutically acceptable. Suitable oils include, for example, mineral oils (especially light or low viscosity mineral oil such as Drakeol® 6VR), vegetable oils (e.g., soybean oil, sunflower oil, corn oil), nut oils (e.g., peanut oil, castor oil, coconut oil), or mixtures thereof. Thus, in an embodiment the hydrophobic substance is vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic substance is Incomplete Freund's Adjuvant (IFA) or Modified Freund's Adjuvant (MFA), a mineral oil-based hydrophobic carrier. In another embodiment, the hydrophobic substance is mannide oleate in mineral oil, such as commercially available Montanide™ ISA 51 (SEPPIC, France). Montanide™ ISA 51 is a mixture of highly purified mineral oil (Drakeol® 6VR) and mannide monooleate that forms a water-in oil (W/O) emulsion when mixed with an aqueous phase in a 1:1 ratio (van Doom 2016). In another embodiment, the hydrophobic substance is mannide oleate in non-mineral oil, such as commercially available Montanide™ ISA 720 (SEPPIC, France). In another embodiment, the hydrophobic phase is MS80 oil which is a mixture of mineral oil (Sigma Aldrich) and sorbitan monooleate (e.g. Span™ 80) (Fluka), the components of which can be purchased separately and mixed prior to use.

The hydrophobic substance may comprise a mixture of an oil with one or more lipids. The term “lipid” has its common meaning in the art in that it is any organic substance or compound that is soluble in nonpolar solvents, but generally insoluble in polar solvents (e.g. water). Lipids are a diverse group of compounds including, without limitation, fats, waxes, sterols, fat-soluble vitamins, monoglycerides, diglycerides, triglycerides and phospholipids. Lipids may be membrane-forming lipids. By “membrane-forming lipids” it is meant that the lipids, alone or together with other lipids and/or stabilizing molecules, are capable of forming a lipid membrane. The lipid membranes may form closed lipid vesicles or any other structure, such as for example lipid sheets. Lipids may be amphiphilic. By “amphiphilic lipids” it is meant that the lipids possess both hydrophilic and hydrophobic (lipophilic) properties. Amphiphilic lipids may act as emulsifiers. Particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids.

In some embodiments, the lipid is a phospholipid or a mixture of phospholipids. Broadly defined, a “phospholipid” is a member of a group of lipid compounds that yield on hydrolysis phosphoric acid, an alcohol, fatty acid, and nitrogenous base. Phospholipids that may be used include, for example and without limitation, those with at least one head group selected from the group consisting of phosphoglycerol, phosphoethanolamine, phosphoserine, phosphocholine (e.g. DOPC; 1,2-Dioleoyl-sn-glycero-3-phosphocholine) and phosphoinositol. In an embodiment, the phospholipid may be phosphatidylcholine or a mixture of lipids comprising phosphatidylcholine. In an embodiment, the lipid may be DOPC (Lipoid GmbH, Germany) or Lipoid S100 lecithin. Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphiphilic. Lecithin, which also may be used, is a natural mixture of phospholipids typically derived from chicken eggs, sheep's wool, soybean and other vegetable sources. All of these and other phospholipids may be used in the practice of the invention. Phospholipids can be purchased, for example, from Avanti lipids (Alabastar, Ala., USA), Lipoid LLC (Newark, N.J., USA) and Lipoid GmbH (Germany), among various other suppliers. Membrane-forming lipids, amphiphilic lipids, and phospholipids may be used in the hydrophobic phase to enhance the solubility or the suspension of an agent in the hydrophobic phase

In some embodiments, a mixture of lipid and cholesterol is mixed with a hydrophobic substance to form the hydrophobic phase. In some embodiments, a mixture of DOPC and unesterined cholesterol is mixed with a hydrophobic substance to form a hydrophobic phase. In other embodiments, a mixture of Lipoid S100 lecithin and unesterified cholesterol is mixed with a hydrophobic substance to form a hydrophobic phase. In some embodiments, the cholesterol is used in an amount equivalent to about 10% of the weight of phospholipid (e.g. in a DOPC:cholesterol ratio of 10:1 w/w). The cholesterol may stabilize the formation of phospholipid vesicle particles.

In some embodiments, the hydrophobic phase comprises a mixture of DOPC and cholesterol that was lyophilized and then reconstituted in mineral oil, mannide oleate in mineral oil (e.g. Montanide™ ISA 51), or MS80 oil. In some embodiments, the hydrophobic phase comprises a mixture of at least one hydrophobic phase agent, DOPC and cholesterol that was lyophilized and then reconstituted in mineral oil, mannide oleate in mineral oil (e.g. Montanide™ ISA 51), or MS80 oil.

Lipid-Based Structures

Within a hydrophobic phase that comprises lipids, there are various lipid-based structures which may form, and the hydrophobic phases disclosed herein may comprise a single type of lipid-based structure or comprise a mixture of different types of lipid-based structures. A lipid-based structure may be a lipid vesicle particle.

In an embodiment, the lipid-based structures may be closed vesicular structures. They are typically spherical or substantially spherical in shape, but other shapes and conformations may be formed and are not excluded. By “substantially spherical” it is meant that the lipid-based structures are close to spherical, but may not be a perfect sphere. Other shapes of the closed vesicular structures include, without limitation, oval, oblong, square, rectangular, triangular, cuboid, crescent, diamond, cylinder or hemisphere shapes. Any regular or irregular shape may be formed. Exemplary embodiments of closed vesicular structures include, without limitation, single layer vesicular structures (e.g. micelles or reverse micelles) and bilayer vesicular structures (e.g. unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, such as with the hydrophobic part of each layer internally oriented toward the center of the bilayer with the hydrophilic part externally oriented. It is expected that in a hydrophobic substance, the opposite configuration is formed, i.e. with the hydrophilic part of each layer internally oriented toward the center of the bilayer with the hydrophobic part externally oriented. The term “multilayer” is meant to encompass any combination of single and bilayer structures. The form adopted may depend upon the specific lipid that is used, and whether the composition is or is not water-free.

The closed vesicular structures may be formed from single layer lipid membranes, bilayer lipid membranes and/or multilayer lipid membranes. The lipid membranes are predominantly comprised of and formed by lipids, but may also comprise additional components. For example, and without limitation, the lipid membrane may include stabilizing molecules to aid in maintaining the integrity of the structure. Any available stabilizing molecule may be used.

In an embodiment, the one or more lipid-based structures are comprised of a single layer lipid assembly. There are various types of these lipid-based structures which may form, and the hydrophobic phases disclosed herein may comprise a single type of lipid-based structure having a single layer lipid assembly or comprise a mixture of different such lipid-based structures.

In an embodiment, the lipid-based structure having a single layer lipid assembly partially or completely surrounds the hydrophobic phase agent. As an example, the lipid-based structure may be a closed vesicular structure surrounding the hydrophobic phase agent. In an embodiment, the hydrophobic part of the lipids in the vesicular structure is oriented outwards toward the hydrophobic substance.

As another example, the one or more lipid-based structures having a single layer lipid assembly may comprise aggregates of lipids with the hydrophobic part of the lipids oriented outwards toward the hydrophobic substance and the hydrophilic part of the lipids aggregating as a core or surrounding the hydrophobic phase agent. These structures do not necessarily form a continuous lipid layer membrane. In an embodiment, they are an aggregate of monomeric lipids.

In an embodiment, the one or more lipid-based structures having a single layer lipid assembly comprise reverse micelles. A typical micelle in a hydrophobic substance forms an inverse/reverse micelle with the hydrophobic parts in contact with the surrounding hydrophobic substance, sequestering the hydrophilic parts in the micelle center. A reverse micelle can package hydrophobic phase agents with hydrophilic affinity within its core (i.e. internal environment).

Without limitation, the size of the lipid-based structures having a single layer lipid assembly is in the range of from 2 nm (20 A) to 20 nm (200 A) in diameter. In an embodiment, the size of the lipid-based structures having a single layer lipid assembly is between about 2 nm to about 10 nm in diameter. In an embodiment, the size of the lipid-based structures having a single layer lipid assembly is about 2 nm, 3 nm, 4 nm, 5 nm, 6 nm, about 7 nm, about 8 nm, about 9 nm, or about 10 nm in diameter. In an embodiment, the maximum diameter of the lipid-based structures is about 4 nm or about 6 nm. In an embodiment, the lipid-based structures of these sizes are reverse micelles.

In an embodiment, the one or more hydrophobic phase agents are inside the lipid-based structures after solubilization in the hydrophobic substance. By “inside the lipid-based structure” it is meant that the hydrophobic phase agent is substantially surrounded by the lipids such that the hydrophilic components of the hydrophobic phase agent are not exposed to the hydrophobic substance. In an embodiment, the hydrophobic phase agent inside the lipid-based structure is predominantly hydrophilic.

In an embodiment, the one or more hydrophobic phase agents are outside the lipid-based structures after solubilization in the hydrophobic substance. By “outside the lipid-based structure”, it is meant that the hydrophobic phase agent is not sequestered within the environment internal to the lipid membrane or assembly. In an embodiment, the hydrophobic phase agent outside the lipid-based structure is predominantly hydrophobic.

Preparing a Hydrophobic Phase

The hydrophobic phase may be composed of at least one hydrophobic phase agent dissolved or suspended in a hydrophobic substance. In some embodiments wherein the at least one hydrophobic phase agent is hydrophobic, the agent may simply be mixed with the hydrophobic substance to form a solution. In some embodiments wherein the at least one hydrophobic phase agent is hydrophilic, the agent may be solubilized or suspended in the hydrophobic substance with an organic solvent and/or a lipid. For example, the at least one hydrophobic phase agent may be mixed with lipids dissolved in an organic solvent prior to mixing with a hydrophobic substance. In another example, the at least one hydrophobic phase agent may be mixed with lipids dissolved in an organic solvent, dried by lyophilization, and then reconstituted in a hydrophobic substance.

The hydrophobic phase may be composed of a composition reconstituted in a hydrophobic substance wherein the composition comprises a mixture of antigen(s) and/or adjuvant(s) in a hydrophobic substance for the purpose of activating an immune response. The composition may further comprise lipids and/or cholesterol to stabilize the antigen(s) and/or adjuvant(s), to promote their solubilisation/suspension in a hydrophobic substance, and/or to promote their uptake by immune cells. The lipids and/or cholesterol in the composition may form lipid structures as described herein to promote the solubility and/or suspension of the antigen(s) and/or adjuvant(s) in the hydrophobic substance. Preferably, the composition is soluble in a hydrophobic substance or is readily suspended in a hydrophobic substance. In some embodiments, the composition for use as a hydrophobic phase in an O/W emulsion according to the present invention comprises a dried mixture of one or more antigen, one or more adjuvant, one or more lipid, and cholesterol that is then reconstituted in a hydrophobic substance or hydrophobic carrier to form the hydrophobic phase. Such compositions and methods for preparing them have been described in WO/2009/146523 and WO/2013/049941.

The hydrophobic phase may be composed of a composition reconstituted in a hydrophobic substance wherein the composition comprises one or more pharmaceutical, therapeutic, or immunomodulatory agent(s) in a hydrophobic substance. The composition may further comprise lipids and/or cholesterol to stabilize the one or more agent(s) and/or to promote its solubilisation/suspension in a hydrophobic substance. The lipids and/or cholesterol in the composition may form lipid structures as described herein to promote the solubility and/or suspension of the agent(s) in the hydrophobic substance. Preferably, the composition is soluble in a hydrophobic substance or is readily suspended in a hydrophobic substance. In some embodiments, the composition for use as a hydrophobic phase in an O/W emulsion according to the present invention comprises a dried mixture of one or more hydrophobic phase agent, one or more lipid, and cholesterol that is then reconstituted in a hydrophobic substance or hydrophobic carrier to form the hydrophobic phase. Such compositions and methods for preparing them have been described in PCT/CA2019/050328.

In some embodiments, the hydrophobic phase is a composition comprising at least one hydrophobic phase agent, lipids, and cholesterol reconstituted in a hydrophobic substance. For preparing the hydrophobic phase in such embodiments, a lipid preparation is prepared by dissolving or hydrating lipids, or a lipid-mixture, in a suitable solvent with gently shaking. The at least one hydrophobic phase agent may then be added to the lipid preparation, either directly (e.g. adding dry hydrophobic phase agent) or by first preparing a stock of the at least one hydrophobic phase agent dissolved in a suitable solvent. Typically, the at least one hydrophobic phase agent is added to, or combined with, the lipid preparation while gently shaking. The hydrophobic agent/lipid composition is then dried to form a dried composition, and the dried composition is reconstituted in a hydrophobic substance. The “suitable solvent” is one that is capable of dissolving the respective component (e.g. lipids, hydrophobic phase agent, or both), and can be determined by the skilled person. In respect of the at least one hydrophobic phase agent, the suitable solvent may be, for example, sodium phosphate solution, sodium acetate solution, sodium hydroxide solution, dimethyl sulfoxide (DMSO), or water. The skilled person can determine other suitable solvents depending on the hydrophobic phase agent to be used. In respect of the lipids, the suitable solvent may be, for example, a polar protic solvent such as an alcohol (e.g. tert-butanol, n-butanol, isopropanol, n-propanol, ethanol or methanol), water, acetate buffer, phosphate buffer, formic acid or chloroform. In an embodiment, the suitable solvent is 40% tertiary-butanol. The skilled person can determine other suitable solvents depending on the lipids to be used.

In another embodiment, for preparing the hydrophobic phase, a lipid-mixture containing DOPC and cholesterol in a 10:1 ratio (w:w) (Lipoid GmBH, Germany) can be dissolved in 40% tertiary-butanol by shaking at 300 RPM at room temperature until dissolved. A stock solution of at least one hydrophobic phase agent can be prepared in DMSO or water and diluted with 40% tertiary-butanol prior to mixing with the dissolved lipid-mixture. Hydrophobic phase agent stock can then be added to the dissolved lipid-mixture with shaking at 300 RPM for about 5 minutes to prepare a composition. The composition can then be freeze-dried to produce a dried composition for storage and later reconstitution with a hydrophobic substance to produce a hydrophobic phase. Optionally, the composition can be freeze-dried with cryoprotectants/bulking agents. Cryoprotectants/bulking agents that can be used include, but are not limited to sugars/polysaccharides such as trehalose, sucrose, mannitol, sorbitol, lactose, maltose, raffinose, maltodextrin, pullulan, inulin, ficoll, carboxymethylcellulose, and hydroxyethyl starch; amino acids such as arginine, histidine, phenylalanine, leucine, and isoleucine; bovine serum albumin; buffer salts such as sodium acetate, sodium phosphate, Tris HCl, HEPES, sodium carbonate, sodium citrate, Tris acetate; and polymers such as poly vinyl pyrrolidone, poly vinyl alcohol, hydroxypropyl-β-cyclodextrin, polyacrylamide, and Carbopol®. The dried composition can then be reconstituted in a hydrophobic substance such as Montanide® ISA 51 VG (SEPPIC, France) to obtain a clear solution. Typically, the dried composition is stored (e.g. at −20° C.) until the time of administration, when the dried composition is reconstituted in the hydrophobic substance to produce the hydrophobic phase for use in forming an emulsion composition as described herein.

In another embodiment, to prepare the hydrophobic phase, the at least one hydrophobic phase agent is dissolved in sodium phosphate buffer with S100 lipids and cholesterol (Lipoid, Germany). These components are then lyophilized to form a dried composition. Prior to use, the dried composition is reconstituted in ISA51 VG oil (SEPPIC, France) to prepare a hydrophobic phase for use in preparing an emulsion composition as described herein.

In another embodiment, to prepare the hydrophobic phase, the at least one hydrophobic phase agent is dissolved in sodium phosphate buffer with DOPC and cholesterol (Lipoid, Germany). These components are then lyophilized to form a dried composition. Prior to use, the dried composition is reconstituted in ISA51 VG oil (SEPPIC, France) to prepare a hydrophobic phase for use in preparing an emulsion composition as described herein.

In some embodiments, the hydrophobic phase is prepared from a composition formed using sized lipid vesicle particles. Methods for preparing such compositions have been described in WO/2019/090411 and WO/2019/010560. As used herein the term “lipid vesicle particle” may be used interchangeably with “lipid vesicle” and refers to a lipid-based structure as described herein.

In some embodiments, the hydrophobic phase is prepared from a freeze-dried composition formed using sized lipid vesicle particles, wherein: (a) lipid vesicle particles having a mean particle size of 120 nm and a polydispersity index (PDI) of ≤0.1 are provided; (b) the lipid vesicle particles are mixed with at least one solubilized hydrophobic phase agent to form a mixture; and (c) the mixture is dried to form a dried composition.

In some embodiments, the hydrophobic phase is prepared from a dried composition formed using sized lipid vesicle particles, wherein: (a) a lipid vesicle particle preparation comprising lipid vesicle particles and at least one solubilized hydrophobic phase agent; (b) the lipid vesicle particle preparation is sized to form a sized lipid vesicle particle preparation comprising sized lipid vesicle particles and the at least one solubilized hydrophobic phase agent, wherein the sized lipid vesicle particles have a mean particle size of 120 nm and a polydispersity index (PDI) of ≤0.1; and (c) the mixture is dried to form a dried composition.

In some embodiments, the hydrophobic phase is prepared from a dried composition formed using sized lipid vesicle particles, wherein: (a) a lipid vesicle particle preparation comprising lipid vesicle particles and at least one solubilized first hydrophobic phase agent; (b) the lipid vesicle particle preparation is sized to form a sized lipid vesicle particle preparation comprising sized lipid vesicle particles and the at least one solubilized first hydrophobic phase agent, wherein the sized lipid vesicle particles have a mean particle size of 120 nm and a polydispersity index (PDI) of ≤0.1; (c) the sized lipid vesicle preparation is mixed with at least one second hydrophobic phase agent, wherein the at least one second hydrophobic phase agent is solubilized in the mixture; and (d) the mixture is dried to form a dried composition.

In embodiments where the hydrophobic phase is prepared from a composition formed using sized lipid vesicle particles, by “solubilized hydrophobic phase agent”, it is meant that the at least one hydrophobic phase agent is dissolved in a solvent. The solvents used in the preparation of the lipid vesicle particle/hydrophobic phase agent mixture must not only be suitable for solubilizing the at least one hydrophobic phase agent in an aqueous environment with the lipids, but must also be suitable for forming a dried lipid/hydrophobic phase agent composition that will be compatible with a hydrophobic substance (e.g. any salts and/or non-volatile solvents should preferably be compatible with the hydrophobic substance). Exemplary solvents that may be used for solubilizing the at least one hydrophobic phase agent include zwitterionic solvents. Non-limiting examples of zwitterionic solvents include HEPES (4-(2-hydroxyethyl)-1-piperazineethanesulfonic acid), MOPS (3-(N-Morpholino) propanesulfonic acid) and MES (2-(N-morpholino)ethanesulfonic acid). Further exemplary solvents for solubilizing the at least one hydrophobic phase agent are aqueous salt solutions. Salts provide useful properties in solubilizing hydrophobic phase agents, and it has also been recognized that certain salts provide stability to the dried lipid/hydrophobic phase agent composition. Non-limiting examples of such solvents include sodium acetate, sodium phosphate, sodium carbonate, sodium bicarbonate, potassium acetate, potassium phosphate, potassium carbonate, and potassium bicarbonate. In an embodiment, the solvent is aqueous sodium acetate. In an embodiment, the sodium acetate may be 25-250 mM sodium acetate having a pH in the range of 6.0-10.5. In an embodiment, the solvent is aqueous sodium phosphate. In an embodiment, the sodium phosphate may be 25-250 mM sodium phosphate having a pH in the range of 6.0-8.0. Depending on the characteristics of the at least one hydrophobic agent it may be advantageous to initially solubilize the at least one hydrophobic phase agent in a mild/weak acidic solvent (e.g. for basic agents) or a mild/weak basic solvent (e.g. for acidic agents). Exemplary acidic solvents that may be used include, without limitation, hydrochloric acid, acetic acid. Exemplary basic solvents that may be used include, without limitation, sodium hydroxide, sodium bicarbonate, sodium acetate and sodium carbonate. For neutral hydrophobic phase agents, an exemplary solvent may be dimethyl sulfoxide (DMSO). In an embodiment, one or more of the hydrophobic phase agents are initially solubilized in a mild/weak basic solvent. In an embodiment, the at least one hydrophobic phase agent is initially solubilized in 50-250 mM sodium hydroxide. Based on the present disclosure, the skilled person could also identify other solvents that may be used that exhibit similar characteristics to those described herein.

In embodiments where the hydrophobic phase is prepared from a composition formed using sized lipid vesicle particles, the sized lipid vesicle particles are prepared by sizing non-sized lipid vesicle particles. To provide a non-sized lipid vesicle particle preparation, lipids in dry powder form may be added to a solution containing at least one solubilized hydrophobic phase agents. In such embodiments, the non-sized lipid vesicle particles are formed in the presence of the at least one hydrophobic phase agent to provide the non-sized lipid vesicle particle preparation. In another embodiment, lipids in dry powder form may be combined with one or more dry hydrophobic phase agent, and the dry combination may be solubilized together in an appropriate solvent. These embodiments may be performed with shaking and/or mixing (e.g. at 300 RPM for about 1 hour). In another embodiment, to provide a non-sized lipid vesicle particle preparation, lipids may first be dissolved and mixed in an organic solvent. In embodiments where different types of lipid are used, this step will allow a homogenous mixture of the lipids to be formed. In an embodiment, these steps may be carried out in chloroform, chloroform:methanol mixtures, tertiary butanol or cyclohexane. In an embodiment, the lipids are prepared at 10-20 mg lipid/mL organic solvent; however, higher or lower concentrations may also be used. After mixing, the organic solvent is removed (e.g. by evaporation) to yield a lipid film. The lipid film may then be frozen and lyophilized to yield a dry lipid film. The dry lipid film may then be hydrated with an aqueous solution containing at least one solubilized hydrophobic phase agent to provide the non-sized lipid vesicle particle preparation. The step of hydration may be performed with shaking and/or mixing (e.g. at 300 RPM for about 1 hour). In yet another embodiment, to provide a non-sized lipid vesicle particle preparation, an aqueous solution of lipids may be combined with a solution containing at least one solubilized hydrophobic phase agents In another embodiment, one or more dry hydrophobic phase agent may be added to, and solubilized in, the aqueous solution of lipids to provide a non-sized lipid vesicle preparation. These embodiments may be performed with shaking and/or mixing (e.g. at 300 RPM for about 1 hour).Various methods may be used to dry the sized lipid vesicle particle preparation which are known in the art. In an embodiment, the drying is performed by lyophilization, spray freeze-drying, or spray drying. The skilled person is well-aware of these drying techniques and how they may be performed.

In embodiments where the hydrophobic phase is prepared from a composition formed using sized lipid vesicle particles, standard procedures for preparing lipid vesicle particles of any size may be employed. For example, conventional liposome forming processes may be used, such as the hydration of solvent-solubilized lipids. Exemplary methods of preparing liposomes are discussed, for example, in Gregoriadis 1990; and Frezard 1999. After the lipid vesicle particles are prepared, the non-sized lipid vesicle particle preparation is subjected to a sizing procedure to obtain lipid vesicle particles having a mean particle size of ≤120 nm and a PDI of ≤0.1. There are various techniques available for sizing lipid vesicle particles (see e.g. Akbarzadeh 2013). For example, in an embodiment, the non-sized lipid vesicle particle preparation may be sized by high pressure homogenization (microfluidizers), sonication or membrane based extrusion. For example, the sized lipid vesicle particles may be prepared by adding the lipids to a suitable solvent (e.g. sodium phosphate, 50 mM, pH 7.0), shaking and/or stirring the lipid mixture (e.g. at 300 RPM for about 1 hour) and using membrane based extrusion to obtain the sized lipid vesicle particles.

In embodiments where the hydrophobic phase is prepared from a composition formed using sized lipid vesicle particles, the sizing of lipid vesicle particles is performed using membrane based extrusion of lipid vesicle particles to obtain the sized lipid vesicle particles having a mean particle size of 120 nm and a PDI of ≤0.1. Exemplary, non-limiting embodiments of membrane based extrusion include passing the non-sized lipid vesicle particle preparation through a 0.2 m polycarbonate membrane and then through a 0.1 m polycarbonate membrane, and then optionally through a 0.08 m polycarbonate membrane. Exemplary, non-limiting protocols may include: (i) passing the non-sized lipid vesicle particle preparation 20-40 times through a 0.2 m polycarbonate membrane, and then 10-20 times through a 0.1 m polycarbonate membrane; or (ii) passing the non-sized lipid vesicle particle preparation 20-40 times through a 0.2 m polycarbonate membrane, then 10-20 times through a 0.1 m polycarbonate membrane, and then 10-20 times through a 0.08 m polycarbonate membrane. The skilled person would be well aware of different membranes and different protocols which may be used to attain the required mean particle size of 120 nm and PDI of ≤0.1. In a particular embodiment, the sizing may be performed by passing a non-sized lipid vesicle particle preparation 25 times through a 0.2 m polycarbonate membrane, and then 10 times through a 0.1 m polycarbonate membrane. In another particular embodiment, the sizing may be performed by passing a non-sized lipid vesicle particle preparation 25 times through a 0.2 m polycarbonate membrane, then 10 times through a 0.1 m polycarbonate membrane, and then 15 times through a 0.08 m polycarbonate membrane.

In embodiments where the hydrophobic phase prepared from a composition formed using sized lipid vesicle particles, the sized lipid vesicle particles may be prepared from a lipid precursor that naturally forms lipid vesicle particles of the required size. For example, and without limitation, the sized lipid vesicle particles may be prepared using Presome® (Nippon Fine Chemical, Japan). Presome® is a dry powder precursor made up of different lipid combinations. Presome® is supplied ready to be wetted in a suitable buffer to prepare liposomes. The liposomes formed from Presome® have an average particle size of about 93 nm, and sizing procedures (e.g. membrane extrusion, high pressure homogenization, etc.) can be used to achieve the required mean particle size of 120 nm and PDI of ≤0.1. In an embodiment, Presome® may for example be wetted in sodium acetate, pH 9.0±0.5 to form liposomes. In an embodiment, the Presome® bulk dry powder may be made from DOPC/cholesterol (10:1 (w/w)) or DOPC alone.

As used herein, polydispersity index (PDI) is a measure of the size distribution of the lipid vesicle particles. It is known in the art that the term “polydispersity” may be used interchangeably with “dispersity”. The PDI can be calculated by determining the mean particle size of the lipid vesicle particles and the standard deviation from that size. There are techniques and instruments available for measuring the PDI of lipid vesicle particles. For example, DLS is a well-established technique for measuring the particle size and size distribution of particles in the submicron size range, with available technology to measure particle sizes of less than 1 nm (LS Instruments, CH; Malvern Instruments, UK).

In embodiments where the hydrophobic phase is prepared from a composition formed using sized lipid vesicle particles, the at least one hydrophobic phase agent is either solubilized in a solvent prior to mixing with the sized lipid vesicle particles or the at least one hydrophobic phase agent is solubilized upon being mixed with the sized lipid vesicle particles. In this latter embodiment, the at least one hydrophobic phase agent may be added as a dry powder to a solution containing the sized lipid vesicle particles or both the sized lipid vesicle particles and dry hydrophobic phase agent may be mixed together in a fresh solvent. When the at least one hydrophobic phase agent is solubilized prior to mixing with the sized lipid vesicle particles, in embodiments where more than one hydrophobic phase agent is used, the individual hydrophobic phase agents may be solubilized together in the same solvent or separate from each other in different solvents. When multiple hydrophobic phase agents are used, some of the agents may be solubilized together and others may be solubilized individually.

In some embodiments, the hydrophobic phases disclosed herein are water-free. As used herein, “water-free” means completely or substantially free of water, i.e. the hydrophobic phases are not emulsions themselves. By “completely free of water” it is meant that the hydrophobic phases contain no water at all. In contrast, the term “substantially free of water” is intended to encompass embodiments where the hydrophobic phases may still contain small quantities of water. For example, individual components of the hydrophobic phase (e.g. lipids and/or agents as described herein) may have small quantities of bound water that may not be completely removed by processes such as lyophilization or evaporation and certain hydrophobic substances may contain small amounts of water dissolved therein. Generally, compositions as disclosed herein that are “substantially free of water” contain, for example, less than about 5%, 4%, 3%, 2%, 1%, 0.5%, 0.1%, 0.05% or 0.01% water on a weight/weight basis of the total weight of the carrier component of the composition.

The hydrophobic phase may further include one or more emulsifiers, such as a surfactant. In a hydrophobic phase, a surfactant may be used to assist in stabilizing lipid-based structures and/or hydrophobic phase agents in the hydrophobic phase. The use of a surfactant may, for example, promote more even distribution of hydrophobic phase agents by reducing surface tensions. In an embodiment, a surfactant may be used when the hydrophobic phase contains several different hydrophobic phase agents (e.g. five or more different peptide antigens) or a relatively high concentration of a hydrophobic phase agent (e.g. ≥5 mg/mg total of agent). The surfactant may be amphiphilic and therefore, the surfactant may include a broad range of compounds. Examples of surfactants which may be used include polysorbates, which are oily liquids derived from polyethylene glycolyated sorbital, and sorbitan esters. Polysorbates may include, for example, sorbitan monooleate. Typical surfactants are well-known in the art and include, without limitation, mannide oleate (Arlacel™ A), lecithin, Tweens™ 20 and 80 (polysorbate 20 and 80), and Spans™ 20, 80, 83 and 85 (sorbitan monolaurate, sorbitan monooleate, sorbitan sesquioleate, and sorbitan trioleate). In an embodiment, the surfactant for use in the hydrophobic phase may be mannide oleate. In an embodiment, the surfactant for use in the hydrophobic phase may be sorbitan monooleate (Span™ 80).

The surfactant is generally pre-mixed with the one or more hydrophobic substance used to form the hydrophobic phase. In some embodiments, a hydrophobic substance which already contains a surfactant may be used. For example, a hydrophobic substance such Montanide™ ISA 51 already contains the surfactant mannide oleate. In other embodiments, the hydrophobic substance may be mixed with a surfactant before combining with the other components of the hydrophobic phase.

Aqueous Phases

An O/W emulsion according to the present invention comprises a continuous aqueous phase. The aqueous phase is immiscible with a hydrophobic phase. The aqueous phase forms an emulsion comprising a dispersion of hydrophobic phase droplets in the aqueous phase. The hydrophobic phase may be dispersed in the aqueous phase to form an emulsion using the techniques disclosed herein, and may further be dispersed using an emulsifier. As used herein, an “aqueous phase” refers to a mixture comprising water and/or one or more aqueous solutions and at least one agent (an aqueous phase agent). An aqueous phase may further comprise other ingredients including, but not limited to organic solvents, emulsifiers, surfactants, lipids, polymers, sugars, buffer salts and amphiphilic substances.

The aqueous phase is composed of water or an aqueous solution. The term “aqueous solution”, as used herein, refers to a solution in which the solvent is water, or in which water is the primary solvent. The aqueous phase may be composed of water, sterile water, de-ionized water, an aqueous solution, or a combination thereof. In some embodiments, the aqueous phase comprises an aqueous solution such as phosphate buffered saline (PBS); glucose solution; saline solution; or buffer solutions containing sodium acetate, sodium carbonate, sodium bicarbonate, potassium acetate, potassium phosphate, potassium carbonate, calcium carbonate potassium bicarbonate, HEPES (4-(2-hydroxyethyl)-1-piperazineethanesulfonic acid), MOPS (3-(N-Morpholino)propanesulfonic acid), MES (2-(N-morpholino)ethanesulfonic acid), bovine serum albumin, sugar alcohols and/or poly ethylene glycols.

In some embodiments, the aqueous phase may further include one of more emulsifiers as described herein. Emulsifiers are added to the aqueous phase (prior to mixing with the hydrophobic phase). In an embodiment, the aqueous phase includes polysorbate 20 (e.g. Tween™ 20) and/or polysorbate 80 (e.g. Tween™ 80) as an emulsifier. In an embodiment, the aqueous phase comprises polysorbate 20 (e.g. Tween™ 20) and/or polysorbate 80 (e.g. Tween™ 80) at concentrations of 0.1%, 0.15%, 0.2%, 0.25%, 0.3%, 0.35%, 0.4%, 0.45%, 0.5%, or more, by weight. In an embodiment, the aqueous phase comprises 0.25% or 0.5% polysorbate (e.g. Tween™ 20), by weight. In another embodiment, the aqueous phase comprises 0.25% or 0.5% polysorbate 80 (e.g. Tween™ 80), by weight.

In some embodiments, the aqueous phase may comprise one or more organic solvents. The organic solvents may be included in the aqueous phase to promote the solubility of one or more aqueous phase agents that are hydrophobic or otherwise poorly soluble in an aqueous solution. For example, one or more aqueous phase agents may be dissolved in an organic solvent, and the organic solvent containing the one or more aqueous phase agents is then mixed with a larger volume of water and/or aqueous solution to form an aqueous phase.

Within an aqueous phase that comprises lipids, there are various lipid-based structures which may form, and the aqueous phases disclosed herein may comprise a single type of lipid-based structure or comprise a mixture of different types of lipid-based structures. In some embodiments, the aqueous phase comprises lipids and/or lipid-based structures in order to promote the solubilisation/suspension of one or more aqueous phase agents in the aqueous phase.

In an embodiment, the lipid-based structure is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). An aqueous phase agent may be contained in the internal environment of the liposome or within the bilayer of the liposome to promote its solubilisation/suspension in the aqueous phase. A general discussion of liposomes can be found in Gregoriadis 1990; and Frezard 1999.

In an embodiment, the lipid-based structure is a single layer lipid assembly. There are various types of these lipid-based structures which may form, and the aqueous phases disclosed herein may comprise a single type of lipid-based structure having a single layer lipid assembly or comprise a mixture of different such lipid-based structures. In an embodiment, the lipid-based structure having a single layer lipid assembly partially or completely surrounds the aqueous phase agent. As an example, the lipid-based structure may be a closed vesicular structure surrounding the aqueous phase agent. In an embodiment, the hydrophilic part of the lipids in the vesicular structure is oriented outwards toward the aqueous phase.

In an embodiment, the one or more lipid-based structures having a single layer lipid assembly comprise micelles. A typical micelle in an aqueous solution forms a micelle with the hydrophilic parts in contact with the surrounding aqueous solution, sequestering the hydrophobic parts in the micelle center. A micelle can package aqueous phase agents with hydrophobic affinity within its core (i.e. internal environment).

In some embodiments, the aqueous phase may comprise a dried preparation of at least one aqueous phase agent that is resuspended in water or an aqueous solution. In some embodiments, the aqueous phase may comprise a dried composition of at least one aqueous phase agent, lipid, and cholesterol, that is resuspended in water or an aqueous solution.

Agents

The compositions according to the present invention are for the delivery of at least two agents to a subject; at least one agent in the hydrophobic phase of the composition (the hydrophobic phase agent) and at least one agent in the aqueous phase of the composition (the aqueous phase agent).

As used herein, “hydrophobic phase agent” refers to an agent that is dissolved or suspended within the hydrophobic phase of an emulsion. A hydrophobic phase agent may itself be hydrophobic (i.e. lipophilic) in which case the hydrophobic phase agent may be soluble in a hydrophobic substance. A hydrophobic phase agent that is hydrophobic may be dissolved or suspended within a hydrophobic substance without the use of a lipid, emulsifier, or amphiphilic substance. A hydrophobic phase agent that is hydrophobic may be incorporated into a hydrophobic phase by mixing said agent with a hydrophobic substance. Alternatively, a hydrophobic phase agent may be hydrophilic (i.e. lipophobic) in which case the hydrophobic phase agent will not be soluble in a hydrophobic substance. A hydrophobic phase agent that is hydrophilic may require the use of a lipid, emulsifier, or amphiphilic substance to solubilize or suspend said agent in a hydrophobic substance. By way of non-limiting example, one or more hydrophobic phase agents that are hydrophilic may be mixed with a phospholipid and cholesterol in an organic solvent, the mixture is then lyophilized, and the lyophilized mixture is then mixed with a hydrophobic substance such that the phospholipid and cholesterol form lipid-based structures as described herein that promote the suspension of the agent in the hydrophobic substance.

As used herein, “aqueous phase agent” refers to an agent that is dissolved or suspended within the aqueous phase of an emulsion. An aqueous phase agent may be hydrophilic (i.e. lipophobic) in which case the aqueous phase agent may be soluble in water or an aqueous solution. An aqueous phase agent that is hydrophilic may be dissolved or suspended within water or an aqueous solution without the use of a lipid, emulsifier, or amphiphilic substance. An aqueous phase agent that is hydrophilic may be incorporated into an aqueous phase by mixing said agent with water or an aqueous solution. Alternatively, an aqueous phase agent may be hydrophobic (i.e. lipophilic) in which case the aqueous phase agent will not be soluble in water or an aqueous solution. An aqueous phase agent that is hydrophobic may require the use of a lipid, emulsifier, organic solvent, or amphiphilic substance to solubilize or suspend said agent in an aqueous solution. By way of non-limiting example, one or more aqueous phase agents that are hydrophobic may be mixed with an aqueous solution containing phospholipid and cholesterol, and the solution is agitated to form lipid-based structures as described herein such that the lipid-based structures conceal the hydrophobic regions of the aqueous phase agent and promote the suspension of the agent in the aqueous solution. Alternatively, one or more aqueous phase agents that are hydrophobic can be solubilized in an organic solvent such as DMSO, ethanol, tert.butanol, DMF, or poly ethylene glycol and then the organic solvent containing the one or more aqueous phase agents can be mixed with water and/or aqueous solution to form an aqueous phase.

Some agents may be amphiphilic, meaning that they possess both a polar or hydrophilic region and a non-polar or hydrophobic region enabling them to interact with both hydrophobic and aqueous phases. Amphiphilic agents may therefore be dissolved or suspended in either a hydrophobic phase or an aqueous phase. An amphiphilic agent that is wholly contained in the hydrophobic phase of an emulsion according to the present invention is a hydrophobic phase agent. An amphiphilic agent that is wholly contained in the aqueous phase of an emulsion according to the present invention is an aqueous phase agent.

The term “agent” includes any substance, drug, molecule, element, compound, or combination thereof that is intended to be delivered to a subject. An agent may be incorporated into a composition of the present invention as a hydrophobic phase agent if it is contained in the hydrophobic phase of the composition, or as an aqueous phase agent is it is contained in the aqueous phase of the composition. An agent can be a natural product, a synthetic compound, or a combination of two or more substances. An agent may be a pharmaceutically or therapeutically active agent or diagnostic agent. An agent may be a small molecule drug; an antibody, an antibody mimetic, or a functional equivalent or functional fragment of any one thereof, an immunomodulatory agent; an antigen; a T helper epitope; an adjuvant; an allergen; a DNA polynucleotide; or an RNA polynucleotide. Particular agents that may be incorporated into a composition according to the present invention in either the hydrophobic phase or the aqueous phase are described in more detail herein.

Small Molecule Drugs

In some embodiments, at least one agent is a small molecule drug. A small molecule drug may be incorporated into a composition according to the present invention as a hydrophobic phase agent and/or an aqueous phase agent. The term “small molecule drug” refers an organic or inorganic compound that may be used to treat, cure, prevent or diagnose a disease, disorder or condition.

As used herein, the term “small molecule” refers to a low molecular weight compound which may be synthetically produced or obtained from natural sources and has a molecular weight of less than 2000 Daltons (Da), less than 1500 Da, less than 1000 Da, less than 900 Da, less than 800 Da, less than 700 Da, less than 600 Da or less than 500 Da.

In an embodiment, the small molecule drug has a molecule weight of between about 100 Da to about 2000 Da; about 100 Da to about 1500 Da; about 100 Da to about 1000 Da; about 100 Da to about 900 Da; about 100 Da to about 800 Da; about 100 Da to about 700 Da; about 100 Da to about 600 Da; or about 100 Da to about 500 Da. In an embodiment, the small molecule drug has a molecular weight of about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da, about 1000 Da, or about 2000 Da. In an embodiment, the small molecule drug may have a size on the order of 1 nm.

In an embodiment, the small molecule drug is a chemically manufactured active substance or compound (i.e. it is not produced by a biological process). Generally, these compounds are synthesized in the classical way by chemical reactions between different organic and/or inorganic compounds. As used herein, the term “small molecule drug” does not encompass larger structures, such as polynucleotides, proteins and polysaccharides, which are made by a biological process.

In an embodiment, as used herein, the term “small molecule” refers to compounds or molecules that selectively bind specific biological macromolecules and act as an effector, altering the activity or function of the target. Thus, in an embodiment, the small molecule drug is a substance or compound that regulates a biological process in the body of a subject, and more particularly within a cell. The small molecule drug may exert its activity in the form in which it is administered, or the small molecule drug may be a prodrug. In this regard, the term “small molecule drug”, as used herein, encompasses both the active form and the prodrug.

The term “prodrug” refers to a compound or substance that, under physiological conditions, is converted into the therapeutically active agent. In an embodiment, a prodrug is a compound or substance that, after administration, is metabolized in the body of a subject into the pharmaceutically active form (e.g. by enzymatic activity in the body of the subject). A common method for making a prodrug is to include selected moieties that are hydrolyzed under physiological conditions to reveal the pharmaceutically active form.

In an embodiment, and without limitation, the small molecule drug is a cytotoxic agent, an anti-cancer agent, an anti-tumor agent, a chemotherapeutic agent, an anti-neoplastic agent, an antiviral agent, an antibacterial agent, an anti-inflammatory agent, an immunomodulatory agent (e.g. an immune enhancer or suppressor), an immune response checkpoint agent, a biological response modifier, a prodrug, a cytokine, a chemokine, a vitamin, a steroid, a ligand, an analgesic, a radiopharmaceutical, a radioisotope or a dye for visual detection.

The small molecule drug may be any of those described herein, or may be a pharmaceutically acceptable salt thereof. As used herein, the term “pharmaceutically acceptable salt(s)” refers to any salt form of an active agent and/or immunomodulatory agent described herein that are safe and effective for administration to a subject of interest, and that possess the desired biological, pharmaceutical and/or therapeutic activity. Pharmaceutically acceptable salts include salts of acidic or basic groups. Pharmaceutically acceptable acid addition salts may include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts may include, but are not limited to, aluminum, calcium, lithium, magnesium, potassium, sodium, zinc, and diethanolamine salts. A review of pharmaceutically acceptable salts can be found, for example, in Berge, 1977.

In an embodiment, the small molecule drug is an agent that interferes with DNA replication. As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The present disclosure encompasses the use of any agent that interferes with DNA replication. Exemplary, non-limiting embodiments of such agents that may be used are described, for example, in WO2014/153636 and in WO2017/190242. In an embodiment, the agent that interferes with DNA replication is an alkylating agent, such as for example a nitrogen mustard alkylating agent such as, for example, cyclophosphamide.

In an embodiment, the small molecule drug is cyclophosphamide, ifosfamide, afosfamide, melphalan, bendamustine, uramustine, palifosfamide, chlorambucil, busulfan, 4-hydroxycyclophosphamide, bis-chloroethylnitrosourea (BCNU), mitomycin C, yondelis, procarbazine, dacarbazine, temozolomide, cisplatin, carboplatin, oxaliplatin, acyclovir, gemcitabine, 5-fluorouracil, cytosine arabinoside, ganciclovir, camptothecin, topotecan, irinotecan, doxorubicin, daunorubicin, epirubicin, idarubicin, etoposide, teniposide, mitoxantrone or pixantrone, or a pharmaceutically acceptable salt of any one thereof.

In an embodiment, the small molecule drug is ifosfamide. Ifosfamide is a nitrogen mustard alkylating agent. The IUPAC name for ifosfamide is N-3-bis(2-chloroethyl)-1,3,2-oxazaphosphinan-2-amide-2-oxide. Ifosfamide is commonly known as Ifex®.

In an embodiment, the small molecule drug is palifosfamide. Palifosfamide is an active metabolite of ifosfamide that is covalently linked to the amino acid lysine for stability. Palifosfamide irreversibly alkylates and cross-links DNA through GC base pairs, resulting in irreparable 7-atom inter-strand cross-links; inhibition of DNA replication and/or cell death. Palifosfamide is also known as Zymafos®.

In an embodiment, the small molecule drug is bendamustine. Bendamustine is another nitrogen mustard alkylating agent. The IUPAC name for Bendamustine is 4-[5-[Bis(2-chloroethyl)amino]-1-methylbenzimidazol-2-yl]butanoic acid, and it is commonly referred to as Treakisym®, Ribomustin®, Levact® and Treanda®.

In an embodiment, the small molecule drug is an immune response checkpoint agent. As used herein, an “immune response checkpoint agent” refers to any compound or molecule that totally or partially modulates (e.g. activates or inhibits) the activity or function of one or more checkpoint molecules (e.g. proteins). Checkpoint molecules are responsible for co-stimulatory or inhibitory interactions of T-cell responses. Checkpoint molecules regulate and maintain self-tolerance and the duration and amplitude of physiological immune responses. Generally, there are two types of checkpoint molecules: stimulatory checkpoint molecules and inhibitory checkpoint molecules.

Stimulatory checkpoint molecules serve a role in enhancing the immune response. Numerous stimulatory checkpoint molecules are known, such as for example and without limitation: CD27, CD28, CD40, CD122, CD137, CD137/4-1BB, ICOS, IL-10, OX40 TGF-beta, TOR receptor, and glucocorticoid-induced TNFR-related protein GITR. In an embodiment, the small molecule drug is an agonist or antagonist of one or more stimulatory checkpoint molecules. In an embodiment, the small molecule drug is an agonist or superagonist of one or more stimulatory checkpoint molecules. The skilled person will be well aware of small molecule drugs that may be used to modulate stimulatory checkpoint molecules.

Inhibitory checkpoint molecules serve a role in reducing or blocking the immune response (e.g. a negative feedback loop). Numerous inhibitory checkpoint proteins are known, such as for example CTLA-4 and its ligands CD80 and CD86; and PD-1 and its ligands PD-L1 and PD-L2. Other inhibitory checkpoint molecules include, without limitation, adenosine A2A receptor (A2AR); B7-H3 (CD276); B7-H4 (VTCN1); BTLA (CD272); killer-cell immunoglobulin-like receptor (KIR); lymphocyte activation gene-3 (LAG3); V-domain Ig suppressor of T cell activation (VISTA) T-cell immunoglobulin domain and mucin domain 3 (TIM-3); and indoleamine 2,3-dioxygenase (IDO), as well as their ligands and/or receptors. In an embodiment, the small molecule drug is an agonist or antagonist of one or more inhibitory checkpoint molecules. In an embodiment, the small molecule drug is an antagonist (i.e. an inhibitor) of one or more inhibitory checkpoint molecules. The skilled person will be well aware of small molecule drugs that may be used to modulate inhibitory checkpoint molecules.

In an embodiment, the small molecule drug is an immune response checkpoint agent that is an inhibitor of Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GAL9, GITR, HVEM, IDO1, IDO2, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-α, VISTA, VTCN1, or any combination thereof.

In an embodiment, the small molecule drug may be epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof.

In an embodiment, the small molecule drug is cyclophosphamide or a pharmaceutically acceptable salt thereof. Cyclophosphamide (N,N-bis(2-chloroethyl)-1,3,2-oxazaphosphinan-2-amine 2-oxide). Cyclophosphamide is also known and referred to under the trade-marks Endoxan®, Cytoxan®, Neosar®, Procytox® and Revimmune®. Cyclophosphamide is a prodrug which is converted to its active metabolites, 4-hydroxy-cyclophosphamide and aldophosphamide, by oxidation by P450 enzymes. Intracellular 4-hydroxy-cyclophosphamide spontaneously decomposes into phosphoramide mustard which is the ultimate active metabolite.

In an embodiment, the small molecule drug is a shuttle, e.g. a molecular shuttle. As used herein, the term “shuttle” refers to a compound or molecule that can transport other molecules or ions from one location to another. Without limitation, the shuttle may be a peptide that is capable of transporting cargo to cells, such as for example a cell-penetrating peptide (CPP), a peptide transduction domain (PTD) and/or a dendritic cell peptide (DCpep). These types of shuttles are described, for example, in Delcroix, 2010; Zhang, 2016; Zahid, 2012; and Curiel, 2004b. The skilled person will be well aware of other shuttles that may be used in the practice of the invention.

The skilled person would be well aware of other small molecule drugs that may be used in the practice of the invention. As an example, and without limitation, reference is made to DrugBank™ (Wishart, 2017). Version 5.0.11 of DrugBank™, released Dec. 20, 2017, contains 10,990 drug entries, including over 2,500 approved small molecule drugs.

Antibodies, Antibody Mimetics or Functional Equivalents or Fragments

In some embodiments, at least one agent is an antibody, a functional equivalent of an antibody or a functional fragment of an antibody. An antibody, a functional equivalent of an antibody or a functional fragment of an antibody may be incorporated into a composition according to the present invention as a hydrophobic phase agent and/or an aqueous phase agent.

Broadly, an “antibody” refers to a polypeptide or protein that consists of or comprises antibody domains, which are understood as constant and/or variable domains of the heavy and/or light chains of immunoglobulins, with or without a linker sequence. In an embodiment, polypeptides are understood as antibody domains if they comprise a beta-barrel sequence consisting of at least two beta-strands of an antibody domain structure connected by a loop sequence. Antibody domains may be of native structure or modified by mutagenesis or derivatization, e.g. to modify binding specificity or any other property.

The term “antibody” refers to an intact antibody. In an embodiment, an “antibody” may comprise a complete (i.e. full-length) immunoglobulin molecule, including e.g. polyclonal, monoclonal, chimeric, humanized and/or human versions having full length heavy and/or light chains. The term “antibody” encompasses any and all isotypes and subclasses, including without limitation the major classes of IgA, IgD, IgE, IgG and IgM, and the subclasses IgG1, IgG2, IgG3, IgG4, IgA1 and IgA2. In an embodiment, the antibody is an IgG. The antibody may be one that is naturally occurring or one that is prepared by any means available to the skilled person, such as for example by using animals or hybridomas, and/or by immunoglobulin gene fragment recombinatorial processes. Antibodies are generally described in, for example, Greenfield, 2014).

In an embodiment, the antibody is in an isolated form, meaning that the antibody is substantially free of other antibodies against a different target antigen and/or comprising a different structural arrangement of antibody domains. In an embodiment, the antibody can be an antibody isolated from the serum sample of mammal. In an embodiment, the antibody is in a purified form, such as provided in a preparation comprising only the isolated and purified antibody as the agent. This preparation may be used in the preparation of a composition of the invention. In an embodiment, the antibody is an affinity purified antibody.

The antibody may be of any origin, including natural, recombinant and/or synthetic sources. In an embodiment, the antibody may be of animal origin. In an embodiment, the antibody may be of mammalian origin, including without limitation human, murine, rabbit and goat. In an embodiment, the antibody may be a recombinant antibody.

In an embodiment, the antibody may be a monoclonal antibody, a polyclonal antibody, a chimeric antibody, a humanized antibody, a human antibody or a fully human antibody. The meaning applied to these terms and the types of antibodies encompassed therein will be well understood by the skilled person.

Briefly, and without limitation, the term “chimeric antibody” as used herein refers to a recombinant protein that contains the variable domains (including the complementarity determining regions (CDRs)) of an antibody derived from one species, such for example a rodent, while the constant domains of the antibody are derived from a different species, such as a human. For veterinary applications, the constant domains of the chimeric antibody may be derived from that of an animal, such as for example a cat or dog.

Without limitation, a “humanized antibody” as used herein refers to a recombinant protein in which the CDRs from an antibody from one species; e.g., a rodent, are transferred from the heavy and light variable chains of the rodent antibody into human heavy and light variable domains, including human framework region (FR) sequences. The constant domains of the humanized antibody are likewise derived from a human antibody.

Without limitation, a “human antibody” as used herein may refer to an antibody obtained from transgenic animals (e.g. mice) that have been genetically engineered to produce specific human antibodies in response to antigenic challenge. In this technique, elements of the human heavy and light chain loci are introduced into strains of mice derived from embryonic stem cell lines that contain targeted disruptions of the endogenous heavy chain and light chain loci. The transgenic animal can synthesize human antibodies specific for human antigens, and the animal can be used to produce human antibody-secreting hybridomas. Methods for obtaining human antibodies from transgenic mice are described e.g. by Green, 1994; Lonberg, 1994; and Taylor, 1994. A fully human antibody also can be constructed by genetic or chromosomal transfection methods, as well as phage display technology, all of which are known in the art. (See, e.g., McCafferty, 1990, for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, 1993. Human antibodies may also be generated by in vitro activated B cells (see, e.g., U.S. Pat. Nos. 5,567,610 and 5,229,275).

As used herein, the term “functional fragment”, with respect to an antibody, refers to an antigen-binding portion of an antibody. In this context, by “functional” it is meant that the fragment maintains its ability to bind to the target antigen. In an embodiment, the binding affinity may be equivalent to, or greater than, that of parent antibody. In an embodiment, the binding affinity may be less than the parent antibody, but nevertheless the functional fragment maintains a specificity and/or selectivity for the target antigen. In an embodiment, in addition to the functional fragment maintaining its ability to bind to the target antigen of the parent antibody, the functional fragment also maintains the effector function of the antibody, if applicable (e.g. activation of the classical complement pathway; antibody dependent cellular cytotoxicity (ADCC); other downstream signalling processes).

Functional fragments of antibodies include, without limitation, a portion of an antibody such as a F(ab′)₂, a F(ab)₂, a Fab′, a Fab, a Fab₂, a Fab₃, a single domain antibody (e.g. a Dab or VHHs) and the like, including half-molecules of IgG4 (van der Neut Kolfschoten, 2007). Regardless of structure, a functional fragment of an antibody binds with the same antigen that is recognized by the intact antibody. The term “functional fragment”, in relation to antibodies, also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”). As used herein, the term “functional fragment” does not include fragments such as Fc fragments that do not contain antigen-binding sites.

Antibody fragments, such as those described herein, can be incorporated into single domain antibodies (e.g. nanobodies), single-chain antibodies, maxibodies, evibodies, minibodies, intrabodies, diabodies, triabodies, tetrabodies, vNAR, bis-scFv and other like structures (see e.g. Hollinger and Hudson, 2005). Antibody polypeptides including fibronectin polypeptide monobodies, also are disclosed in U.S. Pat. No. 6,703,199. Other antibody polypeptides are disclosed in U.S. Patent Publication No. 20050238646.

Another form of a functional fragment is a peptide comprising one or more CDRs of an antibody or one or more portions of the CDRs, provided the resultant peptide retains the ability to bind the target antigen.

A functional fragment may be a synthetic or genetically engineered protein. For example, functional fragments include isolated fragments consisting of the light chain variable region, “Fv” fragments consisting of the variable regions of the heavy and light chains, and recombinant single chain polypeptide molecules which light and heavy regions are connected by a peptide linker (scFv proteins).

As used herein, the terms “antibody” and “functional fragments” of antibodies encompass any derivatives thereof. By “derivatives” it is meant any modification to the antibody or functional fragment, including both modifications that occur naturally (e.g. in vivo) or that are artificially introduced (e.g. by experimental design). Non-limiting examples of such modifications include, for example, sequence modifications (e.g. amino acid substitutions, insertions or deletions), post-translational modifications (e.g. phosphorylation, N-linked glycosylation, O-linked glycosylation, acetylation, hydroxylation, methylation, ubiquitylation, amidation, etc.), or any other covalent attachment or incorporation otherwise of a heterologous molecule (e.g. a polypeptide, a localization signal, a label, a targeting molecule, etc.). In an embodiment, modification of the antibody or functional fragment thereof may be made to generate a bispecific antibody or fragment (i.e. having more than one antigen-binding specificity) or a bifunctional antibody or fragment (i.e. having more than one effector function).

As used herein, a “functional equivalent” in the context of an antibody refers to a polypeptide or other compound or molecule having similar binding characteristics as an antibody to a particular target, but not necessarily being a recognizable “fragment” of an antibody. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody, functional fragment thereof or functional equivalent thereof, is one that is preferentially targeted to lymph nodes or lymphoid cells in a lymphatic tissue to exert its pharmacological and/or therapeutic activity. For example and without limitation, the antibody, functional fragment thereof or functional equivalent thereof may be one that binds to an immune cell in lymph nodes or lymphatic tissue, binds to a desired target expressed or found in lymph nodes or lymphatic tissue (e.g. immune stimulatory or inhibitory molecules) and/or binds to cells, proteins, polypeptides or other targets that may be sequestered or delivered to lymph nodes or lymphatic tissue.

In an embodiment, the antibody, functional fragment thereof or functional equivalent thereof, is one that binds a target on an immune cell, binds a protein or polypeptide produced by an immune cell, or binds a protein or polypeptide that interacts with or exerts a function upon immune cells (e.g. a ligand).

In an embodiment, the antibody, functional fragment thereof or functional equivalent thereof, is one that has an immunomodulatory activity or function. By “immunomodulatory activity or function”, it is meant that the antibody, functional fragment thereof or functional equivalent thereof can enhance (upregulate), suppress (downregulate), direct, redirect or reprogram the immune response.

In an embodiment, the antibody, functional fragment thereof or functional equivalent thereof, is one that binds to a stimulatory checkpoint molecule and/or an inhibitory checkpoint molecule, such has for example, and without limitation, those described herein. In an embodiment, the antibody, functional fragment thereof or functional equivalent thereof, is an agonist or an antagonist of a stimulatory checkpoint molecule and/or an inhibitory checkpoint molecule. In an embodiment, the antibody, functional fragment thereof or functional equivalent thereof, is an antagonist of an inhibitory checkpoint molecule. In an embodiment, the antibody, functional fragment thereof or functional equivalent thereof, is an agonist or super agonist of a stimulatory checkpoint molecule.

In an embodiment, the antibody is an anti-CTLA-4 antibody, a functional fragment thereof or a functional equivalent thereof, or any combination thereof. CTLA-4 (CD152) is a protein receptor that, functioning as an immune checkpoint, downregulates immune responses. In an embodiment, the anti-CTLA-4 antibody inhibits CTLA-4 activity or function, thereby enhancing immune responses. In an embodiment, the anti-CTLA-4 antibody is ipilimumab (Bristol-Myers Squibb), tremelimumab (Pfizer; AstraZeneca) or BN-13 (BioXCell). In another embodiment, the anti-CTLA-4 antibody is UC10-4F10-11, 9D9 or 9H10 (BioXCell) or a human or humanized counterpart thereof.

In an embodiment, the antibody is an anti-PD-1 antibody, a functional fragment thereof or a functional equivalent thereof, or any combination thereof. PD-1 (CD279) is a cell surface receptor that, functioning as an immune checkpoint, downregulates immune responses and promotes self tolerance. In an embodiment, the PD-1 antibody is nivolumab (Opdivo™; Bristol-Myers Squibb). In an embodiment, the PD-1 antibody is pembrolizumab (Keytruda™; Merck). In an embodiment, the PD-1 antibody is pidilizumab (Cure Tech). In an embodiment, the anti-PD-1 antibody is AMP-224 (MedImmune & GSK). In an embodiment, the anti-PD-1 antibody is RMP1-4 or J43 (BioXCell) or a human or humanized counterpart thereof.

In an embodiment, the antibody is an anti-PD-L1 antibody, a functional fragment thereof or a functional equivalent thereof, or any combination thereof. PD-L1 is a ligand of the PD-1 receptor, and binding to its receptor transmits an inhibitory signal that reduces proliferation of CD8+ T cells and can also induce apoptosis. In an embodiment, the PD-L1 antibody is BMS-936559 (Bristol Myers Squibb). In an embodiment, the PD-L1 antibody is atezolizumab (MPDL3280A; Roche). In an embodiment, the PD-L1 antibody is avelumab (Merck & Pfizer). In an embodiment, the PD-L1 antibody is durvalumab (MEDI4736; MedImmune/AstraZeneca).

In other embodiments, and without limitation, the antibody, functional fragment or functional equivalent thereof, may be an anti-PD-1 or anti-PD-L1 antibody, such as for example those disclosed in WO 2015/103602.

In an embodiment, the active agent is an antibody mimetic, a functional equivalent of an antibody mimetic, or a functional fragment of an antibody mimetic.

As used herein, the term “antibody mimetic” refers to compounds which, like antibodies, can specifically and/or selectively bind antigens or other targets, but which are not structurally related to antibodies. Antibody mimetics are usually artificial peptides or proteins, but they are not limited to such embodiments. Typically, antibody mimetics are smaller than antibodies, with a molar mass of about 3-20 kDa (whereas antibodies are generally about 150 kDa). Non-limiting examples of antibody mimetics include peptide aptamers, affimers, affilins, affibodies, affitins, alphabodies, anticalins, avimers, DARPins™, fynomers, Kunitz domain peptides, nanoCLAMPs™, affinity reagents and scaffold proteins. Nucleic acids and small molecules may also be antibody mimetics.

The term “peptide aptamer”, as used herein, refers to peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is a commonly used scaffold protein, the variable peptide loop being inserted within the redox-active site, which is a -Cys-Gly-Pro-Cys- loop in the wild protein, the two cysteins lateral chains being able to form a disulfide bridge. Peptide aptamer selection can be made using different systems, but the most widely used is currently the yeast two-hybrid system.

The term “affimer”, as used herein, represents an evolution of peptide aptamers. An affimer is a small, highly stable protein engineered to display peptide loops which provides a high affinity binding surface for a specific target protein or antigen. Affimers can have the same specificity advantage of antibodies, but are smaller, can be chemically synthesized or chemically modified and have the advantage of being free from cell culture contaminants. Affimers are proteins of low molecular weight, typically 12 to 14 kDa, derived from the cysteine protease inhibitor family of cystatins. The affimer scaffold is a stable protein based on the cystatin protein fold. It displays two peptide loops and an N-terminal sequence that can be randomised to bind different target proteins with high affinity and specificity.

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 kDa (ubiquitin) or 20 kDa (gamma-B crystalline). As used herein, the term affilin also refers to di- or multimerised forms of affilins (Weidle, 2013).

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomization of 13 amino acids located in two alpha-helices involved in the binding activity of the parent protein domain (Feldwisch and Tolmachev, 2012). In an embodiment, it is an Affibody™ sourced from Affibody AB, Stockholm, Sweden.

An “affitin” (also known as nanofitin) is an antibody mimetic protein that is derived from the DNA binding protein Sac7d of Sulfolobus acidocaldarius. Affitins usually have a molecular weight of around 7 kDa and are designed to specifically bind a target molecule by randomising the amino acids on the binding surface (Mouratou, 2012). In an embodiment, the affitin is as described in WO 2012/085861.

The term “alphabody”, as used herein, refers to small 10 kDa proteins engineered to bind to a variety of antigens. Alphabodies are developed as scaffolds with a set of amino acid residues that can be modified to bind protein targets, while maintaining correct folding and thermostability. The alphabody scaffold is computationally designed based on coiled-coil structures, but it has no known counterpart in nature. Initially, the scaffold was made of three peptides that associated non-covalently to form a parallel coiled-coil trimer (US Patent Publication No. 20100305304), but was later redesigned as a single peptide chain containing three α-helices connected by linker regions (Desmet, 2014).

The term “anticalin”, as used herein, refers to an engineered protein derived from a lipocalin (Beste, 1999); Gebauer and Skerra, 2009). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

The term “avimer” (avidity multimers), as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle, 2013).

The term “DARPin™”, as used herein, refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “Fynomer™”, as used herein, refers to a non-immunoglobulin-derived binding polypeptide derived from the human Fyn SH3 domain. Fyn SH3-derived polypeptides are well-known in the art and have been described, e.g. in Grabulovski, 2007; WO 2008/022759; Bertschinger, 2007; Gebauer and Skerra, 2009; and Schlatter, 2012).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6kDA and domains with the required target specificity can be selected by display techniques such as phage display (Weidle, 2013).

The term “monobody” (also referred to as “adnectin”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra, 2009). Monobodies with the desired target specificity can be genetically engineered by introducing modifications in specific loops of the protein. In an embodiment, the monobody is an ADNECTIN™ (Bristol-Myers Squibb, New York, New York).

The term “nanoCLAMP” (CLostridal Antibody Mimetic Proteins), as used herein, refers to affinity reagents that are 15 kDa proteins having tight, selective and gently reversible binding to target molecules. The nanoCLAMP scaffold is based on an IgG-like, thermostable carbohydrate binding module family 32 (CBM32) from a Clostridium perfringens hyaluronidase (Mu toxin). The shape of nanoCLAMPs approximates a cylinder of approximately 4 nm in length and 2.5 nm in diameter, roughly the same size as a nanobody. nanoCLAMPs to specific targets are generated by varying the amino acid sequences and sometimes the length of three solvent exposed, adjacent loops that connect the beta strands making up the beta-sandwich fold, conferring binding affinity and specificity for the target (Suderman, 2017).

The term “affinity reagent”, as used herein, refers to any compound or substance that binds to a larger target molecule to identify, track, capture or influence its activity. Although antibodies and peptide aptamers are common examples, many different types of affinity reagents are available to the skilled person. In an embodiment, the affinity reagent is one that provides a viable scaffold that can be engineered to specifically bind a target (e.g. Top7 is a scaffold engineered specifically to bind CD4; Boschek, 2009).

The term “scaffold proteins”, as used herein, refers polypeptides or proteins that interact and/or bind with multiple members of a signalling pathway. They are regulators of many key signalling pathways. In such pathways, they regulate signal transduction and help localize pathway components. Herein, they are encompassed by the term “antibody mimetics” for their ability to specifically and/or selectively bind target proteins, much like antibodies. In addition to their binding function and specificity, scaffold proteins may also have enzymatic activity. Exemplary scaffold proteins include, without limitation, kinase suppressor of Ras 1 (KNS), MEK kinase 1 (MEKK1), B-cell lymphoma/leukemia 10 (BCL-10), A-kinase-anchoring protein (AKAP), Neuroblast differentiation-associated protein AHNAK, HOMER1, pellino proteins, NLRP family, discs large homolog 1 (DLG1) and spinophillin (PPP1R9B).

Other embodiments of antibody mimetics include, without limitation, Z domain of Protein A, Gamma B crystalline, ubiquitin, cystatin, Sac7D from Sulfolobus acidocaldarius, lipocalin, A domain of a membrane receptor, ankyrin repeat motive, SH3 domain of Fyn, Kunits domain of protease inhibitors, the 10^{th} type III domain of fibronectin, 3- or 4-helix bundle proteins, an armadillo repeat domain, a leucine-rich repeat domain, a PDZ domain, a SUMO or SUMO-like domain, an immunoglobulin-like domain, phosphotyrosine-binding domain, pleckstrin homology domain, or src homology 2 domain.

As used herein, the term “functional fragment”, with respect to an antibody mimetic, refers any portion or fragment of an antibody mimetic that maintains the ability to bind to its target molecule. The functional fragment of an antibody mimetic may be, for example, a portion of any of the antibody mimetics as described herein. In an embodiment, the binding affinity may be equivalent to, or greater than, that of parent antibody mimetic. In an embodiment, the binding affinity may be less than the parent antibody mimetic, but nevertheless the functional fragment maintains a specificity and/or selectivity for the target antigen.

In an embodiment, in addition to the functional fragment of an antibody mimetic maintaining its ability to bind to the target molecule of the parent antibody mimetic, the functional fragment also maintains the effector function of the antibody mimetic, if applicable (e.g. downstream signalling).

As used herein, a “functional equivalent” in the context of an antibody mimetic refers to a polypeptide or other compound or molecule having similar binding characteristics to an antibody mimetic, but not necessarily being a recognizable “fragment” of an antibody mimetic. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹⁰ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody mimetic, functional fragment thereof or functional equivalent thereof, is one that is preferentially targeted to lymph nodes or lymphoid cells in a lymphatic tissue to exert its pharmacological and/or therapeutic activity. For example and without limitation, the antibody mimetic, functional fragment thereof or functional equivalent thereof may be one that binds to an immune cell in lymph nodes or lymphatic tissue, binds to a desired target expressed or found in lymph nodes or lymphatic tissue (e.g. immune stimulatory or inhibitory molecules) and/or binds to cells, proteins, polypeptides or other targets that may be sequestered or delivered to lymph nodes or lymphatic tissue.

In an embodiment, the antibody mimetic, functional fragment thereof or functional equivalent thereof, is one that binds a target on an immune cell, binds a protein or polypeptide produced by an immune cell, or binds a protein or polypeptide that interacts with or exerts a function upon immune cells (e.g. a ligand).

In an embodiment, the antibody mimetic, functional fragment thereof or functional equivalent thereof, is one that has an immunomodulatory activity or function. In an embodiment, the antibody mimetic, functional fragment thereof or functional equivalent thereof, is one that binds to a stimulatory checkpoint molecule and/or an inhibitory checkpoint molecule, such has for example, and without limitation, those described herein. In an embodiment, the antibody mimetic, functional fragment thereof or functional equivalent thereof, is an agonist or an antagonist of a stimulatory checkpoint molecule and/or an inhibitory checkpoint molecule. In an embodiment, the antibody mimetic, functional fragment thereof or functional equivalent thereof, is an antagonist of an inhibitory checkpoint molecule (e.g. CTLA-4, PD-1 or PD-L1). In an embodiment, the antibody mimetic, functional fragment thereof or functional equivalent thereof, is an agonist or super agonist of a stimulatory checkpoint molecule.

Immunomodulatory Agents

In some embodiments, at least one agent is an immunomodulatory agent. An immunomodulatory agent may be incorporated into a composition according to the present invention as a hydrophobic phase agent and/or an aqueous phase agent. As used herein, an “immunomodulatory agent” is a compound or molecule that modulates the activity and/or effectiveness of an immune response. “Modulate”, as used herein, means to enhance (upregulate), suppress (downregulate), direct, redirect or reprogram an immune response. The term “modulate” is not intended to mean activate or induce. By this, it is meant that the immunomodulatory agent modulates (enhances, reduces or directs) an immune response that is activated, initiated or induced by a particular substance (e.g. an antigen), but the immunomodulatory agent is not itself the substance against which the immune response is directed, nor is the immunomodulatory agent derived from that substance.

In an embodiment, the immunomodulatory agent is one that modulates myeloid cells (monocytes, macrophages, dendritic cells, magakaryocytes and granulocytes) or lymphoid cells (T cells, B cells and natural killer (NK) cells). In a particular embodiment, the immunomodulatory agent is one that modulates only lymphoid cells. In an embodiment, the immunomodulatory agent is a therapeutic agent that, when administered, stimulates immune cells to proliferate or become activated.

In an embodiment, the immunomodulatory agent is one that enhances the immune response. The immune response may be one that was previously activated or initiated, but is of insufficient efficacy to provide an appropriate or desired therapeutic benefit. Alternatively, the immunomodulatory agent may be provided in advance to prime the immune system, thereby enhancing a subsequently activated immune response.

In an embodiment, an immunomodulatory agent that enhances the immune response may be selected from cytokines (e.g. certain interleukins and interferons), stem cell growth factors, lymphotoxins, co-stimulatory molecules, hematopoietic factors, colony stimulating factors, erythropoietins, thrombopoietins, and the like, and synthetic analogs of these molecules.

In an embodiment, an immunomodulatory agent that enhances the immune response may be selected from: lymphotoxins, such as tumor necrosis factor (TNF); hematopoietic factors, such as interleukin (IL); colony stimulating factor, such as granulocyte-colony stimulating factor (G-CSF) or granulocyte macrophage-colony stimulating factor (GM-CSF); interferon, such as interferons-alpha, -beta or -lamda; and stem cell growth factor, such as that designated “SI factor”.

Included among the cytokines are growth hormones, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors, such as NGF-beta; platelet-growth factor; transforming growth factors (TGFs), such as TGF-alpha and TGFP; insulin-like growth factor-I and -II; erythropoietin (EPO); osteoinductive factors; interferons, such as interferon-alpha, -beta, and -gamma; colony stimulating factors (CSFs), such as macrophage-CSF (M-CSF); interleukins (ILs), such as IL-1, IL-1alpha, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin and tumor necrosis factor.

In an embodiment, the immunomodulatory agent can be an agent which modulates a checkpoint inhibitor. Immune checkpoint proteins are signaling proteins that play a role in regulating immune response. Some checkpoint inhibitors are receptors located on the surface of a cell that respond to extracellular signaling. For example, many checkpoints are initiated by ligand-receptor interactions. When activated, inhibitory checkpoint proteins produce an anti-inflammatory response that can include activation of regulatory T cells and inhibition of cytotoxic or killer T cells. Cancer cells have been shown to express inhibitory checkpoint proteins as a way to avoid recognition by immune cells. Accordingly, inhibitors of inhibitory checkpoint proteins (i.e. “immune checkpoint inhibitors”) can be used to activate the immune system in an individual to kill cancer cells (see e.g. Pardoll, 2012).

In an embodiment, the immunomodulatory agent is any compound, molecule or substance that is an immune checkpoint inhibitor, including but not limited to, an inhibitor of an immune checkpoint protein selected from Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GAL9, GITR, HVEM, IDO1, IDO2, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-α, VISTA, VTCN1, or any combination thereof.

In an embodiment, the immunomodulatory agent is any compound, molecule or substance that inhibits or blocks CTLA-4. CTLA-4 signaling inhibits T-cell activation, particularly during strong T-cell responses. CTLA-4 blockade using CTLA-4 inhibitors, such as anti-CTLA-4 monoclonal antibodies, has great appeal because suppression of inhibitory signals results in the generation of an antitumor T-cell response. Both clinical and preclinical data indicate that CTLA-4 blockade results in direct activation of CD4+ and CD8+ effector cells, and anti-CTLA-4 monoclonal antibody therapy has shown promise in a number of cancers.

In an embodiment, the immunomodulatory agent is any compound, molecule or substance that inhibits or blocks PD-1. Like CTLA-4 signaling, PD-1/PD-L1 modulates T-cell response. Tregs that express PD-1 have been shown to have an immune inhibitor response and PD-1/PD-L1 expression is thus thought to play a role in self-tolerance. In the context of cancer, tumor cells over express PD-1 and PD-L1 in order to evade recognition by the immune system. Anti-cancer therapy that blocks the PD-L1/PD-1 increases effector T cell activity and decreases suppressive Treg activity which allows recognition and destruction of the tumor by an individual's immune system.

In an embodiment, the immunomodulatory agent is a checkpoint inhibitor. For example, the checkpoint inhibitor may be an antibody that binds to and antagonizes an inhibitory checkpoint protein. Exemplary antibodies include anti-PD1 antibodies (pembrolizumab, nivolumab, pidilizumab, AMP-224, RMP1-4 or J43), anti-PD-L1 antibodies (atezolizumab, avelumab, BMS-936559 or durvalumab), anti-CTLA-4 antibodies (ipilimumab, tremelimumab, BN-13, UC10-4F10-11, 9D9 or 9H10) and the like. In some embodiments, the checkpoint inhibitor may be a small molecule or an RNAi that targets an inhibitory checkpoint protein. In some embodiments, the checkpoint inhibitor may be a peptidomimetic or a polypeptide.

In an embodiment, the immunomodulatory agent may be an immune costimulatory molecule agonist. Immune costimulatory molecules are signaling proteins that play a role in regulating immune response. Some immune costimulatory molecules are receptors located on the surface of a cell that respond to extracellular signaling. When activated, immune costimulatory molecules produce a pro-inflammatory response that can include suppression of regulatory T cells and activation of cytotoxic or killer T cells. Accordingly, immune costimulatory molecule agonists can be used to activate the immune system in an individual to kill cancer cells. Exemplary immune costimulatory molecules include any of CD27, CD28, CD40, CD122, CD137, CD137/4-1BB, ICOS, IL-10, OX40 TGF-beta, TOR receptor, and glucocorticoid-induced TNFR-related protein GITR. For example OX40 stimulation suppresses Treg cell function while enhancing effector T cell survival and activity, thereby increasing anti-tumor immunity. In an embodiment, the immunomodulatory agent is any compound, molecule or substance that is an agonist of a costimulatory immune molecule, including, but not limited to, a costimulatory immune molecule selected from CD27, CD28, CD40, CD122, CD137, CD137/4-1BB, ICOS, IL-10, OX40 TGF-beta, TOR receptor, and glucocorticoid-induced TNFR-related protein GITR. Various immune costimulatory molecule agonists may be used. For example, the immune costimulatory molecule agonist may be an antibody that binds to and activates an immune costimulatory molecule. In further embodiments, the immune costimulatory molecule agonist may be a small molecule that targets and activates an immune costimulatory molecule.

In an embodiment, the immunomodulatory agent is any compound, molecule or substance that is an immunosuppressive agent. By “immunosuppressive agent”, it is meant that the compound, molecule or substance reduces (downregulates) the activity and/or efficacy of the immune response, or directs, redirects or reprograms the immune response in a manner that alleviates an undesired result (e.g. an autoimmune response or allergy). There are many different types of immunosuppressive agent, including, without limitation, calcineurin inhibitors, interleukin inhibitors, selective immunosuppressants and THF-alpha inhibitors.

In an embodiment, and without limitation, the immunomodulatory agent may be an immunosuppressant selected from 5-fluorouracil, 6-thioguanine, adalimumab, anakinra, Atgam, abatacept, alefacept, azathioprine, basiliximab, belatacept, belimumab, benralizumab, brodalumab, canakinumab, certolizumab, chlorambucil, cyclosporine, daclizumab, dimethyl fumerate, dupilumab, eculizumab, efalizumab, ethanercept, everolimus, fingolimod, golimumab, guselkumab, imiquimod, infliximab, ixekizumab, leflunomide, lenlidomide, mechlorethamine, mepolizumab, methotrexate, muromonab-cd3, mycophenolate mofetil, mycophenolic acid, natallizumab, omalizumab, pomalidomide, pimecrolimus, reslizumab, rilonacept, sarilumab, secukinumab, siltuximab, sirolimus, tacrolimus, teriflunomide, thalidomide, Thymoglobulin, tocilizumab, ustekinumab and vedolizumab.

In an embodiment, the immunomodulatory agent is any compound, molecule or substance that is an immunosuppressive cytotoxic drug. In an embodiment, the immunosuppressive cytotoxic drug is a glucocorticoid, a cytostatic (e.g. alkylating agents, antimetabolites), an antibody, a drug acting on immunophilins, an interferon, an opioid, or a TNF binding protein. Immunosuppressive cytotoxic drugs include, without limitation, nitrogen mustards (e.g. cyclophosphamide), nitrosoureas, platinum compounds, folic acid analogs (e.g. methotrexate), purine analogs (e.g. azathioprine and mercaptopurine), pyrimidine analogs (e.g. fluorouracil), protein synthesis inhibitors, cytotoxic antibiotics (e.g. dactinomycin, anthracyclines, mitomycin C, bleomycin and mithramycin), cyclosporine, tacrolimus, sirolimus/rapamycin, everolimus, prednisone, dexamethasone, hydrocortisone, mechlorethamine, clorambucil, mycopholic acid, fingolimod, myriocin, infliximab, etanercept, or adalimumab.

In an embodiment, the immunomodulatory agent is an anti-inflammatory agent. In one embodiment, the anti-inflammatory agent is a non-steroidal anti-inflammatory agent. In an embodiment, the non-steroidal anti-inflammatory agent is a Cox-1 and/or Cox-2 inhibitor. In an embodiment, anti-inflammatory agent includes, without limitation, aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In an embodiment, the anti-inflammatory agent is a steroidal anti-inflammatory agent. In an embodiment, the steroidal anti-inflammatory agent is a corticosteroid.

In an embodiment, the immunomodulatory agent is an anti-rheumatic agent. In an embodiment, the anti-rheumatic agent is a non-steroidal anti-inflammatory agent. In an embodiment, the anti-rheumatic agent is a corticosteroid. In an embodiment, the corticosteroid is prednisone or dexamethasone. In an embodiment, the anti-rheumatic agent is a disease modifying anti-rheumatic drug. In an embodiment, disease modifying anti-rheumatic drugs include but are not limited to chloroquine, hydroxychloroquine, methotrexate, sulfasalazine, cyclosporine, azathioprine, cyclophosphamide, azathioprine, sulfasalazine, penicillamine, aurothioglucose, gold sodium thiomalate, or auranofin. In an embodiment, the anti-rheumatic agent is an immunosuppressive cytotoxic drug. In one embodiment, immunosuppressive cytotoxic drugs include but are not limited to methotrexate, mechlorethamine, cyclophosphamide, chlorambucil or azathioprine.

The skilled person will be well aware of other immunomodulatory agents encompassed within the above. Notably, the term “immunomodulatory agent”, as used herein, does not encompass compounds or compositions that function to enhance the immunogenicity of an antigen by prolonging the exposure of the antigen to immune cells (i.e. by a delivery platform, such as Freund's™ complete or incomplete adjuvant, Montanide™ ISA, or other oil-based substances).

Antigens

In some embodiments, at least one agent is an antigen. An antigen may be incorporated into a composition according to the present invention as a hydrophobic phase agent and/or an aqueous phase agent. As used herein, the term “antigen” refers to any substance or molecule that can bind specifically to components of the immune system. In some embodiments, suitable antigens are those that are capable of inducing or generating an immune response in a subject. An antigen that is capable of inducing an immune response is said to be immunogenic, and may also be called an immunogen. Thus, as used herein, the term “antigen” includes immunogens and the terms may be used interchangeably unless specifically stated otherwise.

As used herein, the term “peptide antigen” is an antigen as defined above that is a protein or a polypeptide. In an embodiment, the peptide antigen may be derived from a microorganism, such as for example a live, attenuated, inactivated or killed bacterium, virus or protozoan, or part thereof. In an embodiment, the peptide antigen may be derived from an animal, such as for example a human, or an antigen that is substantially related thereto.

As used herein, the term “derived from” encompasses, without limitation: a peptide antigen that is isolated or obtained directly from an originating source (e.g. a subject); a synthetic or recombinantly generated peptide antigen that is identical or substantially related to a peptide antigen from an originating source; or a peptide antigen which is made from a peptide antigen of an originating source or a fragment thereof. When it is stated that a peptide antigen is “from” a source, the term “from” may be equated with “derived from”. The term “substantially related”, in this context, means that the peptide antigen may have been modified by chemical, physical or other means (e.g. sequence modification), but that the resultant product remains capable of generating an immune response to the original peptide antigen and/or to the disease or disorder associated with the original antigen. “Substantially related” includes variants and/or derivatives of the native peptide antigen. An antigen that is “derived from” an organism may also be said to be “associated with” said organism.

In an embodiment, the peptide antigen can be isolated from a natural source. In some embodiments, the peptide antigen may be purified to be from about 90% to about 95% pure, from about 95% to about 98% pure, from about 98% to about 99% pure, or greater than 99% pure.

In an embodiment, the peptide antigen can be recombinantly generated, such as for example by expression in vitro or in vivo.

In an embodiment, the peptide antigen is a synthetically produced polypeptide based on a sequence of amino acids of a native target protein. The peptide antigen can be synthesized, in whole or in part, using chemical methods well known in the art (see e.g., Caruthers 1980, Horn 1980, Banga 1995). For example, peptide synthesis can be performed using various solid-phase techniques (see e.g., Roberge 1995, Merrifield 1997) and automated synthesis may be achieved, e.g., using the ABI 431A Peptide Synthesizer (Perkin Elmer) in accordance with the instructions provided by the manufacturer.

In the context of peptide antigens, many different types of peptide modifications are known in the art and may be used in the practice of the present invention. For example, and without limitation, the peptide antigen may be modified to improve its solubility, stability and/or immunogenicity. Non-limiting examples of modifications that may be made include N-terminal modifications, C-terminal modifications, amidation, acetylation, peptide cyclization by creating disulfide bridges, phosphorylation, methylation, conjugation to other molecules (e.g. BSA, KLH, OVA), PEGylation and the inclusion of unnatural amino acids.

In an embodiment, the modification may be an amino acid sequence modification, e.g. deletion, substitution or insertion. The substitution may be a conservative amino acid substitution or a non-conservative amino acid substitution. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

In an embodiment, the peptide antigen may be 5 to 120 amino acids in length, 5 to 100 amino acids in length, 5 to 75 amino acids in length, 5 to 50 amino acids in length, 5 to 40 amino acids in length, 5 to 30 amino acids in length, 5 to 20 amino acids in length or 5 to 10 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. In an embodiment, the peptide antigen is 8 to 40 amino acids in length. In an embodiment, the peptide antigen is 9 or 10 amino acids in length.

In an embodiment, the peptide antigen comprises at least one B cell epitope, at least one CTL epitope, or any combination thereof.

B cell epitopes are epitopes recognized by B cells and by antibodies. B cell peptide epitopes are typically at least five amino acids, more often at least six amino acids, still more often at least seven or eight amino acids in length, and may be continuous (“linear”) or discontinuous (“conformational”); the latter being formed, for example, by the folding of a protein to bring non-contiguous parts of the primary amino acid sequence into physical proximity.

CTL epitopes are molecules recognized by cytotoxic T lymphocytes. CTL epitopes are typically presented on the surface of an antigen-presenting cell, complexed with MHC molecules. As used herein, the term “CTL epitope” refers to a peptide which is substantially the same as a natural CTL epitope of an antigen. The CTL epitope may be modified as compared to its natural counterpart, such as by one or two amino acids. Unless otherwise stated, reference herein to a CTL epitope is to an unbound molecule that is capable of being taken up by cells and presented on the surface of an antigen-presenting cell.

The CTL epitope should typically be one that is amendable to recognition by T cell receptors so that a cell-mediated immune response can occur. For peptides, CTL epitopes may interact with class I or class II MHC molecules. CTL epitopes presented by MHC class I molecules are typically peptides between 8 and 15 amino acids in length, and more often between 9 and 11 amino acids in length. CTL epitopes presented by MHC class II molecules are typically peptides between 5 and 24 amino acids in length, and more often between 13 and 17 amino acids in length. If the antigen is larger than these sizes, it will be processed by the immune system into fragments of a size more suitable for interaction with MHC class I or II molecules. Therefore, CTL epitopes may be part of larger peptide antigen than those mentioned above.

Many CTL epitopes are known. Several techniques of identifying additional CTL epitopes are recognized in the art. In general, these involve preparing a molecule which potentially provides a CTL epitope and characterizing the immune response to that molecule.

In an embodiment, the peptide antigen may be one that is associated with cancer, an infectious disease, an addiction disease, or any other disease or disorder.

Viruses, or parts thereof, from which a peptide antigen may be derived include for example, and without limitation, Cowpoxvirus, Vaccinia virus, Pseudocowpox virus, herpes virus, Human herpesvirus 1, Human herpesvirus 2, Cytomegalovirus, Human adenovirus A-F, Polyomavirus, human papillomavirus (HPV), Parvovirus, Hepatitis A virus, Hepatitis B virus, Hepatitis C virus, human immunodeficiency virus (HIV), Seneca Valley virus (SVV), Orthoreovirus, Rotavirus, Ebola virus, parainfluenza virus, influenza virus (e.g. H5N1 influenza virus, influenza A virus, influenza B virus, influenza C virus), Measles virus, Mumps virus, Rubella virus, Pneumovirus, respiratory syncytial virus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus, varicella, severe acute respiratory syndrome coronavirus (SARS-CoV), severe acute respiratory syndrome coronavirus 2 (SARS-CoV-2), and Middle East respiratory syndrome-related coronavirus (MERS-CoV).

In an embodiment, the peptide antigen is derived from HPV. In an embodiment, the HPV peptide antigen is one that is associated with HPV-related cervical cancer or HPV-related head and neck cancer. In an embodiment, the peptide antigen is a peptide comprising the sequence RAHYNIVTF (HPV16E7 (H-2Db) peptide 49-57; R9F; SEQ ID NO: 1). In an embodiment, the peptide antigen is a peptide comprising the sequence YMLNLGPET (HPV Y9T peptide; SEQ ID NO: 2).

In an embodiment, the peptide antigen is derived from HIV. In an embodiment, the HIV peptide antigen may be derived from the V3 loop of HIV-1 gp120. In an embodiment, the HIV peptide antigen may be RGP10 (RGPGRAFVTI; SEQ ID NO: 3). RGP10 may be purchased from Genscript (Piscataway, N.J.). In another embodiment, the peptide antigen may be AMQ9 (AMQMLKETI; SEQ ID NO: 4). AMQ9 peptide is the immunodominant MHC class I epitope of gag for mice of the H-2Kd haplotype. AMQ9 may also be purchased from Genscript.

In an embodiment, the peptide antigen is derived from RSV. The RSV virion, a member of the genus Paramyxoviridae, is composed of a single strand of negative-sense RNA with 15,222 nucleotides. The nucleotides encode three transmembrane surface proteins (F, G and small hydrophobic protein or SH), two matrix proteins (M and M2), three nucleocapsid proteins (N, P and L), and two non-structural proteins (NS1 and NS2). In an embodiment, the peptide antigen may be derived from any one or more of the RSV proteins. In a particular embodiment, the peptide antigen may be derived from the SH protein of RSV or any other paramyxovirus, or a fragment thereof. The RSV peptide antigen may be any one or more of the RSV peptides described or disclosed in WO 2012/065997.

The SH protein, present in a number of paramyxoviruses (Collins 1990), is a transmembrane protein with an ectodomain or “extracellular” component. The human RSV SH protein contains 64 amino acids (Subgroup A; SEQ ID NO: 5) and 65 amino acids (Subgroup B; SEQ ID NO: 6) and is highly conserved.

In an embodiment, the peptide antigen comprises or consists of the ectodomain of the SH protein (SHe) of a paramyxovirus, or a fragment or modified variant thereof. In an embodiment, SHe is derived from bovine RSV. In another embodiment, SHe is derived from a subgroup A human RSV strain or a subgroup B human RSV strain. In an embodiment, the peptide antigen is Subgroup A human RSV SHe (NKLCEYNVFHNKTFELPRARVNT; SEQ ID NO: 7). In an embodiment, the peptide antigen is Subgroup B human RSV SHe (NKLSEHKTFCNKTLEQGQMYQINT; SEQ ID NO: 8).

In an embodiment, the RSV peptide antigen may be in monomeric form, dimeric form, or another oligomeric form, or any combination thereof. In an embodiment, the peptide antigen comprising SHe A and/or SHe B is a monomer (e.g. a single polypeptide). In another embodiment, the peptide antigen comprising SHe A and/or SHe B is dimer (e.g. two separate polypeptides dimerized). Means of dimerization are known in the art. An exemplary procedure is to dissolve the RSV SHe peptide antigens in a mixture of 10% DMSO/0.5% acetic acid in water (w/w) and heat at 37° C. overnight.

In an embodiment, the peptide antigen derived from RSV may comprise or consist of any one or more of the following:

| Name | Sequence | SEQ ID NO |
|---|---|---|
| SheA | NKLCEYNVFHNKTFELPRARVNT | 7 |
| (monomer) | | |
| SheA | NKLCEYNVFHNKTFELPRARVNT | 7 |
| (dimer) | \| | 7 |
| | NKLCEYNVFHNKTFELPRARVNT | |
| SHeA | NKLSEYNVFHNKTFELPRARVNT | 9 |
| (C45S) | | |
| bSheA | NKLCDLNDHHTNSLDIRTRLRNDTQLITRAHEGSINQSSN | 10 |
| (monomer) | | |
| bSheA | NKLCDLNDHHTNSLDIRTRLRNDTQLITRAHEGSINQSSN | 10 |
| (dimer) | \| | 10 |
| | NKLCDLNDHHTNSLDIRTRLRNDTQLITRAHEGSINQSSN | |
| bSHeA | NKLSDLNDHHTNSLDIRTRLRNDTQLITRAHEGSINQSSN | 11 |
| (C45S) | | |
| SheB | NKLSEHKTFCNKTLEQGQMYQINT | 8 |
| (monomer) | | |
| SheB | NKLSEHKTFCNKTLEQGQMYQINT | 8 |
| (dimer) | \| | 8 |
| | NKLSEHKTFCNKTLEQGQMYQINT | |
| SHeB | NKLSEHKTFSNKTLEQGQMYQINT | 12 |
| (C51S) | | |
| SHeB | NKLCEHKTFSNKTLEQGQMYQINT | 13 |
| (C45S) | | |
| SHe B | NKLCEHKTFSNKTLEQGQMYQINT | 13 |
| (S45C) | \| | 13 |
| | NKLCEHKTFSNKTLEQGQMYQINT | |
| L-SHe B | CGGGSNKLSEHKTFSNKTLEQGQMYQINT | 14 |
| (C51S) | \| | 14 |
| | CGGGSNKLSEHKTFSNKTLEQGQMYQINT | |

As described for example in WO 2012/065997, the SHe peptide antigen may be genetically or chemically linked to a carrier. Exemplary embodiments of carriers suitable for presentation of peptide antigens are known in the art, some of which are described in WO 2012/065997. In another embodiment, the SHe peptide antigen may be linked to a sized lipid vesicle particle as described herein or a structure formed therefrom or resulting therefrom as a result of the methods of manufacture.

In another embodiment, the peptide antigen is derived from an influenza virus. Influenza is a single-stranded RNA virus of the family Orthomyxoviridae and is often characterized based on two large glycoproteins on the outside of the viral particle, hemagglutinin (HA) and neuraminidase (NA). Numerous HA subtypes of influenza A have been identified (Kawaoka 1990; Webster 1983). In some embodiments, the antigen may be derived from the HA or NA glycoproteins. In a particular embodiment, the antigen may be recombinant HA antigen (H5N1, A/Vietnam/1203/2004; Protein Sciences; USA), such as derived from the sequence found under GenBank Accession number AY818135 or any suitable sequence variant thereof.

Bacteria, or parts thereof, from which a peptide antigen may be derived include for example, and without limitation, Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0157: H7, Enterohemorrhagic Escherichia coli, Enterotoxigenic Escherichia coli, Haemophilus influenzae, Helicobacter pylon, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

In an embodiment, the peptide antigen is derived from a Bacillus anthracis. Without limitation, the peptide antigen may for example be derived from anthrax recombinant protective antigen (rPA) (List Biological Laboratories, Inc.; Campbell, Calif.) or anthrax mutant recombinant protective antigen (mrPA). rPA has an approximate molecular weight of 83,000 daltons (Da) and corresponds a cell binding component of the three-protein exotoxin produced by Bacillus anthracis. The protective antigen mediates the entry of anthrax lethal factor and edema factor into the target cell. In some embodiments, the antigen may be derived from the sequence found under GenBank Accession number P13423, or any suitable sequence variant thereof.

Protozoa, or parts thereof, from which a peptide antigen may be derived include for example, and without limitation, the genus Plasmodium (Plasmodium falciparum, Plasmodium malariae, Plasmodium vivax, Plasmodium ovale or Plasmodium knowlesi), which causes malaria.

In an embodiment, the peptide antigen is derived from a Plasmodium species. For example, and without limitation, the peptide antigen may be derived from the circumsporozoite protein (CSP), which is a secreted protein of the sporozoite stage of the malaria parasite (Plasmodium sp.). The amino-acid sequence of CSP consists of an immunodominant central repeat region flanked by conserved motifs at the N- and C-termini that are implicated in protein processing as the parasite travels from the mosquito to the mammalian vector. The structure and function of CSP is highly conserved across the various strains of malaria that infect humans, non-human primates and rodents. In an embodiment, the peptide antigen derived from CSP is a malaria virus-like particle (VLP) antigen which comprises circumsporozoite T and B cell epitopes displayed on the woodchuck hepatitis virus core antigen.

In another embodiment, the peptide antigen may be derived from a cancer or tumor-associated protein, such as for example, a membrane surface-bound cancer antigen.

In an embodiment, the cancer may be one that is caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. Thus, in an embodiment, the peptide antigen may be derived from a virus that is linked to the development of cancer.

In an embodiment, the peptide antigen is a cancer-associated antigen. Many cancer or tumor-associated proteins are known in the art such as for example, and without limitation, those described in WO 2016/176761. The methods, preparations, compositions, uses and kits disclosed herein may use or comprise any peptide antigen of a cancer-associated antigen, or a fragment or modified variant thereof.

In a particular embodiment, the peptide antigen is one or more survivin antigens. Survivin, also called baculoviral inhibitor of apoptosis repeat-containing 5 (BIRC5), is a protein involved in the negative regulation of apoptosis. It has been classed as a member of the family of inhibitors of apoptosis proteins (IAPs). Survivin is a 16.5 kDa cytoplasmic protein containing a single BIR motif and a highly charged carboxy-terminal coiled region instead of a RING finger. The gene coding for survivin is nearly identical to the sequence of Effector Cell Protease Receptor-1 (EPR-1), but oriented in the opposite direction. The coding sequence for the survivin (Homo sapiens) is 429 nucleotides long including stop codons (SEQ ID NO: 15). The encoded protein survivin (Homo sapiens) is 142 amino acids long (SEQ ID NO: 16).

In an embodiment, the peptide antigen is any peptide, polypeptide or variant thereof derived from a survivin protein, or a fragment thereof. In an embodiment, the peptide antigen may be a survivin antigen, such as for example and without limitation, those disclosed in WO 2016/176761.

In an embodiment, the survivin peptide antigen may comprise the full length survivin polypeptide. Alternatively, the survivin peptide antigen may be a survivin peptide comprising a fragment of any length of the survivin protein. Exemplary embodiments include a survivin peptide that comprises at least 5, 6, 7, 8, 9, 10, 11, 12, 13, 14, 15, 16, 17, 18, 19 or 20 amino acid residues. In specific embodiments, the survivin peptide consists of a heptapeptide, an octapeptide, a nonapeptide, a decapeptide or an undecapeptide, consisting of 7, 8, 9, 10, 11 consecutive amino acid residues of the survivin protein (e.g. SEQ ID NO: 16), respectively. Particular embodiments of the survivin antigen include survivin peptides of about 9 or 10 amino acids.

Survivin peptide antigens also encompass variants and functional equivalents of natural survivin peptides. Variants or functional equivalents of a survivin peptide encompass peptides that exhibit amino acid sequences with differences as compared to the specific sequence of the survivin protein, such as one or more amino acid substitutions, deletions or additions, or any combination thereof. The difference may be measured as a reduction in identity as between the survivin protein sequence and the survivin peptide variant or survivin peptide functional equivalent. In an embodiment, the peptide antigen may include any one or more of the survivin peptides, survivin peptide variants or survivin peptide functional equivalents disclosed in WO 2004/067023; WO 2006/081826 or WO 2016/176761. In a particular embodiment, the survivin peptide antigen may be any one or more of: FEELTLGEF (SEQ ID NO: 17); FTELTLGEF (SEQ ID NO: 18); LTLGEFLKL (SEQ ID NO: 19); LMLGEFLKL (SEQ ID NO: 20); RISTFKNWPF (SEQ ID NO: 21); RISTFKNWPK (SEQ ID NO: 22); STFKNWPFL (SEQ ID NO: 23); LPPAWQPFL (SEQ ID NO: 24).

In an embodiment, the peptide antigen is a self-antigen. As is well-known in the art, a self-antigen is an antigen that originates from within the body of a subject. The immune system is usually non-reactive against self-antigens under normal homeostatic conditions. These types of antigens therefore pose a difficulty in the development of targeted immune therapies. In an embodiment, the peptide antigen is a self-antigen or a fragment or modified variant thereof.

In an embodiment, the peptide antigen is a neoantigen. As used herein, the term “neoantigen” refers to a class of tumor antigens which arise from tumor-specific mutations in an expressed protein. The neoantigen can be derived from any cancer, tumor or cell thereof. In the context of neoantigens, the term “derived from” as used herein encompasses, without limitation: a neoantigen that is isolated or obtained directly from an originating source (e.g. a subject); a synthetic or recombinantly generated neoantigen that is identical in sequence to a neoantigen from an originating source; or a neoantigen which is made from a neoantigen of an originating source or a fragment thereof. The mutations in the expressed protein that create the neoantigen may be patient-specific. By “patient-specific”, it is meant that the mutation(s) are unique to an individual subject. However, it is possible that more than one subject will share the same mutation(s). Thus, a “patient-specific” mutation may be shared by a small or large sub-population of subjects.

A neoantigen may comprise one or more neoepitopes. As used herein, the term “epitope” refers to a peptide sequence which can be recognized by the immune system, specifically by antibodies, B cells or T cells. A “neoepitope” is an epitope of a neoantigen which comprises a tumor-specific mutation as compared to the native amino acid sequence. Generally, neoepitopes may be identified by screening neoantigens for anchor residues that have the potential to bind patient HLA. The neoepitopes are normally ranked using algorithms, such as NetMHC, that can predict peptide binding to HLA.

A “T-cell neoepitope” is to be understood as meaning a mutated peptide sequence which can be bound by the MHC molecules of class I or II in the form of a peptide-presenting MHC molecule or MHC complex. The T-cell neoepitope should typically be one that is amenable to recognition by T cell receptors so that a cell-mediated immune response can occur. A “B-cell neoepitope” is to be understood as meaning a mutated peptide sequence which can be recognized by B cells and/or by antibodies.

In some embodiments, at least one of the neoepitopes of the neoantigen is a patient-specific neoepitope. As used herein, by “patient-specific neoepitope”, it is meant that the mutation(s) in the neoepitope are unique to an individual subject. However, it is possible that more than one subject will share the same mutation(s). Thus, a “patient-specific neoepitope” may be shared by a small or large sub-population of subjects.

In an embodiment, the neoantigen may be selected from mutated somatic proteins of a cancer using selection algorithms such as NetMHC which look for motifs predicted to bind to MHC class I and/or MHC class II proteins. In an embodiment, the neoantigen may be derived from a mutated gene or protein that has previously been associated with cancer phenotypes, such as for example tumor suppressor genes (e.g. p53); DNA repair pathway proteins (e.g. BRCA2) and oncogenes. Exemplary embodiments of genes which often contain mutations giving rise to cancer phenotypes are described, for example, in Castle 2012. The skilled person will be well aware of other mutated genes and/or proteins associated with cancer, and these are available from other literature sources. In some embodiments, the neoantigen may comprise or consist of the neoantigens disclosed by Castle 2012. Castle 2012 does not provide the actual sequences of the neoantigens, but does provide the gene ID and location of the mutated peptide from which the actual sequence can be identified using e.g. the PubMed database available online from the National Center for Biotechnology Information (NCBI).

In an embodiment, the neoantigen may be one or more of the Mutl-50 neoantigens disclosed in Table 1 of Castle 2012, or a neoantigen of the same or related protein (e.g. a human homologue). In an embodiment, the neoantigen may be one or more of Mut25 (STANYNTSHLNNDVWQIFENPVDWKEK; SEQ ID NO: 25), Mut30 (PSKPSFQEFVDWENVSPELNSTDQPFL; SEQ ID NO: 26) and Mut44 (EFKHIKAFDRTFANNPGPMVVFATPGM; SEQ ID NO: 27), or a neoantigen of the same or related protein (e.g. a human homologue).

In a particular embodiment, the peptide antigen is one or more melanoma-associated antigen 9 (MAGE-A9) antigen. MAGE-A9 is a protein belonging to the melanoma-associated antigens (MAGE) group of proteins that are expressed in a wide variety of malignant tumors. In some embodiments, the peptide antigen is any peptide, polypeptide or variant thereof derived from MAGE-A9 protein, or a fragment thereof. In an embodiment, the MAGE-A9 peptide antigen may comprise the full length MAGE-A9 polypeptide. Alternatively, the MAGE-A9 peptide antigen may be a MAGE-A9 peptide comprising a fragment of the MAGE-A9 protein. In a particular embodiment, the MAGE-A9 peptide antigen may be any one or more of

(SEQ ID NO: 35)
KVAELVHFL;
(SEQ ID NO: 36)
GLMGAQEPT;
(SEQ ID NO: 37)
ALSVMGVYV;
(SEQ ID NO: 38)
FLWGSKAHA.

T-Helper Epitopes

In some embodiments, at least one agent is a T-helper epitope. A T-helper epitope may be incorporated into a composition according to the present invention as a hydrophobic phase agent and/or an aqueous phase agent. In some embodiments, a T-helper epitope is used when at least one other agent is an antigen.

T-helper epitopes are a sequence of amino acids (natural or non-natural amino acids) that have T-helper activity. T-helper epitopes are recognised by T-helper lymphocytes, which play an important role in establishing and maximising the capabilities of the immune system, and are involved in activating and directing other immune cells, such as for example cytotoxic T lymphocytes. A T-helper epitope can consist of a continuous or discontinuous epitope. Hence not every amino acid of a T-helper is necessarily part of the epitope.

Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis 1988, Demotz 1989, Chong 1992). The T-helper domain of the subject peptides may have from about 10 to about 50 amino acids, and more particularly about 10 to about 30 amino acids. When multiple T-helper epitopes are present, then each T-helper epitope acts independently.

In another embodiment, the T-helper epitope may be a T-helper epitope analog or a T-helper segment. T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

In a particular embodiment, the T-helper epitope may be the modified Tetanus toxin peptide A16L (amino acids 830 to 844; AQYIKANSKFIGITEL; SEQ ID NO: 28), with an alanine residue added to its amino terminus to enhance stability (Slingluff 2001).

Other sources of T-helper epitopes which may be used include, for example, hepatitis B surface antigen helper T cell epitopes, pertussis toxin helper T cell epitopes, measles virus F protein helper T cell epitope, Chlamydia trachomitis major outer membrane protein helper T cell epitope, diphtheria toxin helper T cell epitopes, Plasmodium falciparum circumsporozoite helper T cell epitopes, Schistosoma mansoni triose phosphate isomerase helper T cell epitopes, Escherichia coli TraT helper T cell epitopes and immune-enhancing analogs and segments of any of these T-helper epitopes.

In some embodiments, the T-helper epitope may be a universal T-helper epitope. A universal T-helper epitope as used herein refers to a peptide or other immunogenic molecule, or a fragment thereof, that binds to a multiplicity of MHC class II molecules in a manner that activates T cell function in a class II (CD4+ T cells)-restricted manner. An example of a universal T-helper epitope is PADRE (pan-DR epitope) comprising the peptide sequence AKXVAAWTLKAAA, wherein X may be cyclohexylalanyl (SEQ ID NO: 29). PADRE specifically has a CD4+ T-helper epitope, that is, it stimulates induction of a PADRE-specific CD4+ T-helper response.

In addition to the modified tetanus toxin peptide A16L mentioned earlier, Tetanus toxoid has other T-helper epitopes that work in the similar manner as PADRE. Tetanus and diphtheria toxins have universal epitopes for human CD4+ cells (Diethelm-Okita 2000). In another embodiment, the T-helper epitope may be a tetanus toxoid peptide such as F21E comprising the peptide sequence FNNFTVSFWLRVPKVSASHLE (amino acids 947 to 967; SEQ ID NO: 30).

In some embodiments, the T-helper epitope may form part of a peptide antigen described herein. In particular, if the peptide antigen is of sufficient size, it may contain an epitope that functions as a T-helper epitope. In other embodiments, the T-helper epitope is a separate molecule from the peptide antigen. In other embodiments, the T-helper epitope may be fused to the peptide antigen.

In an embodiment, the R9F peptide antigen (SEQ ID NO: 1) is fused to the PADRE T-helper epitope (SEQ ID NO: 29) to form a fusion peptide (FP; SEQ ID NO: 34).

Many other T-helper epitopes are known in the art, and any of these T-helper epitopes may be used in the practice of the methods, compositions, uses, and kits disclosed herein.

Adjuvants

In some embodiments, at least one agent is an adjuvant. An adjuvant may be incorporated into a composition according to the present invention as a hydrophobic phase agent and/or an aqueous phase agent. An “adjuvant”, as used herein, refers to a compound or substance that enhances the immune response to an antigen.

A large number of adjuvants have been described and are known to those skilled in the art. Exemplary adjuvants include, without limitation, alum, other compounds of aluminum, Bacillus of Calmette and Guerin (BCG), TiterMax™, Ribi™, Freund's Complete Adjuvant (FCA), CpG-containing oligodeoxynucleotides (CpG ODN), lipid A mimics or analogs thereof, lipopeptides and polyL:C polynucleotides.

In an embodiment, the adjuvant is a CpG ODN. CpG ODNs are DNA molecules that contain one or more unmethylated CpG motifs (consisting of a central unmethylated CG dinucleotide plus flanking regions). An exemplary CpG ODN is 5′-TCCATGACGTTCCTGACGTT-3′ (SEQ ID NO: 31). The skilled person can readily select other appropriate CpG ODNs on the basis of the target species and efficacy.

In an embodiment, the adjuvant is a polyL:C polynucleotide. PolyI:C polynucleotides are polynucleotide molecules (either RNA or DNA or a combination of DNA and RNA) containing inosinic acid residues (I) and cytidylic acid residues (C), and which induce the production of inflammatory cytokines, such as interferon. In an embodiment, the polyI:C polynucleotide is double-stranded. In such embodiments, they may be composed of one strand consisting entirely of cytosine-containing nucleotides and one strand consisting entirely of inosine-containing nucleotides, although other configurations are possible. For instance, each strand may contain both cytosine-containing and inosine-containing nucleotides. In some instances, either or both strands may additionally contain one or more non-cytosine or non-inosine nucleotides.

It has been reported that polyL:C can be segmented every 16 residues without an effect on its interferon activating potential (Bobst 1981). Furthermore, the interferon inducing potential of a polyL:C molecule mismatched by introducing a uridine residue every 12 repeating cytidylic acid residues (Hendrix 1993), suggests that a minimal double stranded polyL:C molecule of 12 residues is sufficient to promote interferon production. Others have also suggested that regions as small as 6-12 residues, which correspond to 0.5-1 helical turn of the double stranded polynucleotide, are capable of triggering the induction process (Greene 1978). If synthetically made, polyL:C polynucleotides are typically about 20 or more residues in length (commonly 22, 24, 26, 28 or 30 residues in length). If semi-synthetically made (e.g. using an enzyme), the length of the strand may be 500, 1000 or more residues.

Accordingly, as used herein, a “polyL:C”, “polyL:C polynucleotide” or “polyL:C polynucleotide adjuvant” is a double- or single-stranded polynucleotide molecule (RNA or DNA or a combination of DNA and RNA), each strand of which contains at least 6 contiguous inosinic or cytidylic acid residues, or 6 contiguous residues selected from inosinic acid and cytidylic acid in any order (e.g. IICIIC or ICICIC), and which is capable of inducing or enhancing the production of at least one inflammatory cytokine, such as interferon, in a mammalian subject. PolyI:C polynucleotides will typically have a length of about 8, 10, 12, 14, 16, 18, 20, 22, 24, 25, 26, 28, 30, 35, 40, 45, 50, 55, 60, 65, 70, 75, 80, 85, 90, 95, 100, 150, 200, 250, 300, 500, 1000 or more residues. Preferred polyL:C polynucleotides may have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides.

Each strand of a double-stranded polyL:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g. uridine), provided there is at least one contiguous region of 6 I, 6 C or 6 I/C residues as described above. Typically, each strand of a polyL:C polynucleotide will contain no more than 1 non-I/C residue per 6 I/C residues, more preferably, no more than 1 non-I/C residue per every 8, 10, 12, 14, 16, 18, 20, 22, 24, 26, 28 or 30 I/C residues.

The inosinic acid or cytidylic acid (or other) residues in the polyL:C polynucleotide may be derivatized or modified as is known in the art, provided the ability of the polyL:C polynucleotide to promote the production of an inflammatory cytokine, such as interferon, is retained. Non-limiting examples of derivatives or modifications include e.g. azido modifications, fluoro modifications, or the use of thioester (or similar) linkages instead of natural phosphodiester linkages to enhance stability in vivo. The polyL:C polynucleotide may also be modified to e.g. enhance its resistance to degradation in vivo by e.g. complexing the molecule with positively charged poly-lysine and carboxymethylcellulose, or with a positively charged synthetic peptide.

In an embodiment, the polyL:C polynucleotide may be a single-stranded molecule containing inosinic acid residues (I) and cytidylic acid residues (C). As an example, and without limitation, the single-stranded polyL:C may be a sequence of repeating dIdC. In a particular embodiment, the sequence of the single-stranded polyL:C may be a 26-mer sequence of (IC)₁₃, i.e. ICICICICICICICICICICICICIC (SEQ ID NO: 32). As the skilled person will appreciate, due to their nature (e.g. complementarity), it is anticipated that these single-stranded molecules of repeating dIdC would naturally form homodimers, so they are conceptually similar to polyI/polyC dimers.

In an embodiment, the polyL:C polynucleotide adjuvant is a traditional form of polyL:C with an approximate molecular weight of 989,486 Daltons, containing a mixture of varying strand lengths of polyI and polyC of several hundred base pairs (Thermo Scientific; USA).

In an embodiment, the adjuvant may be one that activates or increases the activity of TLR2. As used herein, an adjuvant which “activates” or “increases the activity” of a TLR2 includes any adjuvant, in some embodiments a lipid-based adjuvant, which acts as a TLR2 agonist. Further, activating or increasing the activity of TLR2 encompasses its activation in any monomeric, homodimeric or heterodimeric form, and particularly includes the activation of TLR2 as a heterodimer with TLR1 or TLR6 (i.e. TLR1/2 or TLR2/6). Exemplary embodiments of an adjuvant that activates or increases the activity of TLR2 include lipid-based adjuvants, such as those described in WO2013/049941.

In an embodiment, the adjuvant may be a lipid-based adjuvant, such as disclosed for example in WO2013/049941. In an embodiment, the lipid-based adjuvant is one that comprises a palmitic acid moiety such as dipalmitoyl-S-glyceryl-cysteine (PAM2Cys) or tripalmitoyl-S-glyceryl-cysteine (PAM3Cys). In an embodiment, the adjuvant is a lipopeptide. Exemplary lipopeptides include, without limitation, PAM2Cys-Ser-(Lys)4 (SEQ ID NO: 33) or PAM3Cys-Ser-(Lys)4 (SEQ ID NO: 33).

In an embodiment, the adjuvant is PAM3Cys-SKKKK (EMC Microcollections, Germany; SEQ ID NO: 33) or a variant, homolog and analog thereof. The PAM2 family of lipopeptides has been shown to be an effective alternative to the PAM3 family of lipopeptides.

In an embodiment, the adjuvant may be a lipid A mimic or analog adjuvant, such as for example those disclosed in WO2016/109880 and the references cited therein. In a particular embodiment, the adjuvant may be JL-265 or JL-266 as disclosed in WO2016/109880.

Further examples of adjuvants that may be used include, without limitation, chemokines, colony stimulating factors, cytokines, 1018 ISS, aluminum salts, Amplivax, AS04, AS15, ABM2, Adjumer, Algammulin, AS01B, AS02 (SBASA), ASO2A, BCG, Calcitriol, Chitosan, Cholera toxin, CP-870,893, CpG, polyL:C, CyaA, DETOX (Ribi Immunochemicals), Dimethyldioctadecylammonium bromide (DDA), Dibutyl phthalate (DBP), dSLIM, Gamma inulin, GM-CSF, GMDP, Glycerol, IC30, IC31, Imiquimod, ImuFact IMP321, IS Patch, ISCOM, ISCOMATRIX, JuvImmune, LipoVac, LPS, lipid core protein, MF59, monophosphoryl lipid A and analogs or mimics thereof, Montanide™ IMS1312, Montanide™ based adjuvants (e.g. Montanide™ ISA-51, -50, -70, and -720), OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, Saponin, synthetic polynucleotides, Zymosan, pertussis toxin.

Allergens

In some embodiments, at least one agent is an allergen. An allergen may be incorporated into a composition according to the present invention as a hydrophobic phase agent and/or an aqueous phase agent. The allergen, fragment, analog or variant thereof may be obtained from a natural source or be synthetically prepared.

An “allergen”, as used herein, refers to any substance that can cause an allergy. The allergen may be derived from, without limitation, cells, cell extracts, proteins, polypeptides, peptides, polysaccharides, polysaccharide conjugates, peptide and non-peptide mimics of polysaccharides and other molecules, small molecules, lipids, glycolipids, and carbohydrates of plants, animals, fungi, insects, food, drugs, dust, and mites. Allergens include but are not limited to environmental aeroallergens; plant pollens (e.g. ragweed/hayfever); weed pollen allergens; grass pollen allergens; Johnson grass; tree pollen allergens; ryegrass; arachnid allergens (e.g. house dust mite allergens); storage mite allergens; Japanese cedar pollen/hay fever; mold/fungal spore allergens; animal allergens (e.g. dog, guinea pig, hamster, gerbil, rat, mouse, etc., allergens); food allergens (e.g. crustaceans; nuts; citrus fruits; flour; coffee); insect allergens (e.g. fleas, cockroach); venoms: (Hymenoptera, yellow jacket, honey bee, wasp, homet, fire ant); bacterial allergens (e.g. streptococcal antigens; parasite allergens such as Ascaris antigen); viral allergens; drug allergens (e.g. penicillin); hormones (e.g. insulin); enzymes (e.g. streptokinase); and drugs or chemicals capable of acting as incomplete antigens or haptens (e.g. the acid anhydrides and the isocyanates).

DNA or RNA Polynucleotide

In some embodiments, at least one agent is a DNA polynucleotide or RNA polynucleotide. A DNA or RNA polynucleotide may be incorporated into a composition according to the present invention as a hydrophobic phase agent and/or an aqueous phase agent. In some embodiments, the DNA or RNA polynucleotide encodes a polypeptide. In some embodiments, the DNA or RNA polynucleotide encodes one or more of the peptide antigens described herein. In some embodiments, the DNA or RNA polynucleotide encodes a polypeptide to be expressed in vivo in a subject.

As used herein, the “DNA or RNA polynucleotide” encompasses a chain of nucleotides of any length (e.g. 9, 12, 15, 18, 21, 24, 27, 30, 60, 90, 120, 150, 300, 600, 1200, 1500 or more nucleotides) or number of strands (e.g. single-stranded or double-stranded). Polynucleotides may be DNA (e.g. genomic DNA, cDNA, plasmid DNA) or RNA (e.g. mRNA) or combinations thereof. The polynucleotide may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability, solubility or transcriptional/translational activity of the polynucleotide.

The polynucleotide may be used in various forms. In an embodiment, a naked polynucleotide may be used, either in linear form, or inserted into a plasmid, such as an expression plasmid. In other embodiments, a live vector such as a viral vector or bacterial vector may be used.

Depending on the nature of the polynucleotide and the intended use, one or more regulatory sequences that aid in transcription of DNA into RNA and/or translation of RNA into a polypeptide may be present. For example, if it is intended or not required that the polynucleotide be transcribed or translated, such regulatory sequences may be absent. In some instances, such as in the case of a polynucleotide that is a messenger RNA (mRNA) molecule, regulatory sequences relating to the transcription process (e.g. a promoter) are not required, and protein expression may be effected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject. The choice of expression cassette depends on the subject as well as the features desired for the expressed polypeptide. Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

Promoters suitable for expression of polynucleotides in a wide range of host systems are well-known in the art. Promoters suitable for expression of polynucleotides in mammals include those that function constitutively, ubiquitously or tissue-specifically. Examples of non-tissue specific promoters include promoters of viral origin. Examples of viral promoters include Mouse Mammary Tumor Virus (MMTV) promoter, Human Immunodeficiency Virus Long Terminal Repeat (HIV LTR) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters. Compatibility of viral promoters with certain polypeptides is a consideration since their combination may affect expression levels. It is possible to use synthetic promoter/enhancers to optimize expression (see e.g. US patent publication 2004/0171573). An example of a tissue-specific promoter is the desmin promoter which drives expression in muscle cells (Li 1989; Li & Paulin 1991; and Li & Paulin 1993). Other examples include artificial promoters such as a synthetic muscle specific promoter and a chimeric muscle-specific/CMV promoter (Li 1999; Hagstrom 2000).

As noted above, the polynucleotide of interest, together with any necessary regulatory sequences, may be delivered naked, e.g. either alone or as part of a plasmid, or may be delivered in a viral or bacterial or bacterial vector. Whether a plasmid-type vector, or a bacterial or viral vector is used, it may be desirable that the vector be unable to replicate or integrate substantially in the subject. Such vectors include those whose sequences are free of regions of substantial identity to the genome of the subject, as to minimize the risk of host-vector recombination. One way to do this is to use promoters not derived from the recipient genome to drive expression of the polypeptide of interest. For example, if the recipient is a mammal, the promoter is preferably non-mammalian derived though it should be able to function in mammalian cells, e.g. a viral promoter.

Viral vectors that may be used to deliver the polynucleotide include e.g. adenoviruses and poxviruses. Useful bacterial vectors include e.g. Shigella, Salmonella, Vibrio cholerae, Lactobacillus, Bacille bilie de Calmette-Guerin (BCG), and Streptococcus. An example of an adenovirus vector, as well as a method for constructing an adenovirus vector capable of expressing a polynucleotide, is described in U.S. Pat. No. 4,920,209. Poxvirus vectors include vaccinia and canary pox virus, described in U.S. Pat. Nos. 4,722,848 and 5,364,773, respectively. Also see, e.g., Tartaglia 1992 for a description of a vaccinia virus vector and Taylor 1995 for a reference of a canary pox. Poxvirus vectors capable of expressing a polynucleotide of interest may be obtained by homologous recombination as described in Kieny 1984, so that the polynucleotide is inserted in the viral genome under appropriate conditions for expression in mammalian cells.

With respect to bacterial vectors, non-toxicogenic Vibrio cholerae mutant strains that are useful for expressing a foreign polynucleotide in a host are known. Mekalanos 1983 and U.S. Pat. No. 4,882,278 describe strains which have a substantial amount of the coding sequence of each of the two ctxA alleles deleted so that no functional cholerae toxin is produced. WO 92/11354 describes a strain in which the irgA locus is inactivated by mutation; this mutation can be combined in a single strain with ctxA mutations. WO 94/01533 describes a deletion mutant lacking functional ctxA and attRSl DNA sequences. These mutant strains are genetically engineered to express heterologous proteins, as described in WO 94/19482. Attenuated Salmonella typhimurium strains, genetically engineered for recombinant expression of heterologous proteins are described in Nakayama 1988 and WO 92/11361. Other bacterial strains which may be used as vectors to express a foreign protein in a subject are described for Shigella flexneri in High 1992 and Sizemore 1995; for Streptococcus gordonii in Medaglini 1995; and for Bacille Calmette Guerin in Flynn 1994, WO 88/06626, WO 90/00594, WO 91/13157, WO 92/01796, and WO 92/21376. In bacterial vectors, the polynucleotide of interest may be inserted into the bacterial genome or remain in a free state as part of a plasmid.

In some embodiments, the RNA polynucleotide does not encode a polypeptide and is an antisense RNA. As used herein, an “antisense RNA” is any single-stranded RNA that is complementary to a messenger RNA (mRNA). The antisense RNA may exhibit 100% complementarity to the mRNA or less than 100% complementarity so long as the antisense RNA is still able to inhibit translation of the mRNA by base pairing to it, thereby obstructing the translation machinery. In an embodiment, the antisense RNA is highly structured, comprised of one or more stem-and-loop secondary structures, flanked or separated by single-stranded (unpaired) regions. In some embodiments, tertiary structures, such as pseudoknots, may form between two or more secondary structural elements.

In some embodiments, the RNA polynucleotide does not encode a polypeptide and is an interfering RNA, such as a small interfering RNA (siRNA), a microRNA (miRNA) or a small hairpin RNA (shRNA). RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. siRNA is a class of double-stranded RNA molecules that are typically 20-25 base pairs in length. It interferes with the expression of specific genes with complementary nucleotide sequences by degrading mRNA after transcription, thereby preventing translation. The natural structure of siRNA is typically a short 20-25 double-stranded RNA with two overhanging nucleotides on each end. The Dicer enzyme catalyzes production of siRNAs from long dsRNAs and small hairpin RNAs (shRNA). shRNA is an artificial RNA molecule with a tight hairpin turn. The design and production of siRNA molecules, and mechanisms of action, are known in the art. miRNAs resemble siRNAs, except miRNAs derive from regions of RNA transcripts that fold back on themselves to form short hairpins, whereas siRNAs derive from longer double-stranded RNA. In an embodiment, the therapeutic agent may be any one or more of these interfering RNAs (siRNA, miRNA or shRNA). The interfering RNA should be one which is capable of decreasing or silencing (preventing) the expression of a gene/mRNA of its endogenous cellular counterpart. In an embodiment, the interfering RNA derived from a naturally occurring interfering RNA. In an embodiment, the interfering RNA is synthetically produced. In an embodiment, the therapeutic agent may be an antagomir. Antagomirs (also known as anti-miRs or blockmirs) are synthetically engineered oligonucleotides that silence endogenous miRNA. It is unclear how antagomirization (the process by which an antagomir inhibits miRNA activity) operates, but it is believed to inhibit by irreversibly binding the miRNA. Because of the promiscuity of microRNAs, antagomirs could affect the regulation of many different mRNA molecules. Antagomirs are designed to have a sequence that is complementary to an mRNA sequence that serves as a binding site for microRNA.

Compositions

The compositions of the present invention comprise an emulsion of a hydrophobic phase comprising at least one hydrophobic phase agent, with the hydrophobic phase being emulsified in an aqueous phase comprising at least one aqueous phase agent.

A composition as disclosed herein may be administered to a subject in a therapeutically effect amount. As used herein, a “therapeutically effective amount” means an amount of the composition or agent contained therein effective to provide a therapeutic, prophylactic or diagnostic benefit to a subject, and/or an amount sufficient to activate or modulate an immune response in a subject. As used herein, to “activate” or “induce” an immune response means to to elicit and/or potentiate an immune response. Inducing an immune response encompasses instances where the immune response is initiated, enhanced, elevated, improved or strengthened to the benefit of the host relative to the prior immune response status. As used herein, to “modulate” an immune response is distinct and different from activating an immune response. By “modulate”, it is meant that the active agents and/or immunomodulatory agents herein enhance or suppress an immune response that is activated by other mechanisms or compounds (e.g. by an antigen or immunogen).

In some embodiments, a therapeutically effective amount of the composition is an amount capable of inducing a clinical response in a subject in the treatment of a particular disease or disorder. Determination of a therapeutically effective amount of the composition is well within the capability of those skilled in the art, especially in light of the disclosure provided herein. The therapeutically effective amount may vary according to a variety of factors such as the subject's condition, weight, sex and age.

In some embodiments, one or more components of the emulsion composition are provided as dried preparations or dried compositions for reconstitution in an aqueous solution or a hydrophobic substance. Various methods may be used to produce dried preparations or dried compositions which are known in the art. In an embodiment, the drying is performed by lyophilization, spray freeze-drying, or spray drying. The skilled person is well-aware of these drying techniques and how they may be performed. In an embodiment, the drying is performed by lyophilization. As used herein, “lyophilization”, “lyophilized” and “freeze-drying” are used interchangeably. As is well known in the art, lyophilization works by freezing the material and then reducing the surrounding pressure to allow the volatile solvent (e.g. water) in the material to sublime directly from the solid phase to the gas phase.

As used herein, the term “dried preparation” or “dried composition” does not necessarily mean that the preparation or composition is completely dry. For example, depending on the solvent or solvents used in the methods disclosed herein, it is possible that a small component of volatile and/or non-volatile material will remain in the dried preparation or dried composition. In an embodiment, the non-volatile material will remain. By “dried preparation” or “dried composition”, it is meant that the preparation or composition no longer contains substantial quantities of water. The process used to dry the preparation or composition should be capable of removing substantially all water from the sized lipid vesicle particle/therapeutic agent mixture. Thus, in an embodiment, the dried preparation or dried composition is completely free of water. In another embodiment, the dried preparation or dried composition may contain a residual moisture content based on the limitations of the drying process (e.g. lyophilization). This residual moisture content will typically be less than 2%, less than 1%, less than 0.5%, less than 0.25%, less than 0.1%, less than 0.05% or less by weight of the dried preparation. This residual moisture content will not be more than 5% by weight of the dried preparation as this would result in a product that is not clear.

When needed, dried preparations or dried compositions can be reconstituted in a suitable solvent, carrier, or liquid. As used herein, by “reconstituted”, it is meant that a dried preparation or dried composition is brought into solution or suspension by the addition of a suitable solvent, solution, carrier, or liquid to the dried preparation or dried composition. As used herein, the terms “reconstituted” and “resuspended” may be used interchangeably. For example, a suitable volume of a hydrophobic substance (e.g. mannide oleate in mineral oil) may be added to a dried composition of lipid, cholesterol, and at least one hydrophobic agent to reconstitute the dried composition. In another example, a suitable volume of water may be added to a dried preparation of at least one aqueous phase agent to reconstitute the dried preparation. During reconstitution, the dried preparation or dried composition can be left to soak for a period of time in the solvent, carrier, or liquid and/or mixed by agitation until the dried preparation or dried composition is fully dissolved or suspended.

Kits

The compositions disclosed herein are optionally provided to a user as a kit. In an embodiment, the kit is for preparing a composition for the treatment, prevention and/or diagnosis of a disease, disorder or condition. In an embodiment, the kit is for preparing a composition for inducing an antibody and/or CTL immune response. In an embodiment, the kit is for preparing a composition for the delivery of at least two active, pharmaceutical, or therapeutic agents. In an embodiment, the kit is for preparing a composition for providing a therapeutic combination therapy.

In some embodiments, ingredients of the composition are provided in the kit as dried preparations or dried compositions for resuspension in a hydrophobic substance or an aqueous solution as disclosed herein. The provision of dried preparations or dried compositions may be advantageous for the storage and/or stability of the ingredients.

In an embodiment, a kit of the present disclosure comprises a container comprising a dried preparation of at least one hydrophobic phase agent. In an embodiment, a kit of the present disclosure comprises a container comprising a dried composition of at least one hydrophobic phase agent, lipids, and cholesterol. In such embodiments, a hydrophobic substance is required for resuspending the dried preparation or dried composition. The hydrophobic substance may be provided in the kit in a separate container, supplied separately, or is already in possession by the end user.

In an embodiment, a kit of the present disclosure comprises a container comprising an aqueous phase, wherein the aqueous phase comprises water and/or an aqueous solution, and at least one aqueous phase agent.

In an embodiment, a kit of the present disclosure comprises a container comprising a dried preparation of at least one aqueous phase agent. In such embodiments, water and/or an aqueous solution is required for resuspending the dried preparation. The water and/or aqueous solution may be provided in the kit in a separate container, supplied separately, or is already in possession by the end user.

The kits can further comprise one or more additional reagents, packaging materials, and an instruction set or user manual detailing preferred methods of using the kit components. In some embodiments, the kits comprise one or more syringes for mixing and/or administering the composition. In such embodiments, the kits may further contain a connector to connect the syringes. In an embodiment, the containers are vials.

Methods and Uses

The compositions disclosed herein may find application in any instance in which it is desired to administer at least two active, pharmaceutical, or therapeutic agents to a subject. The subject may be a vertebrate, such as a fish, bird or mammal. In an embodiment, the subject is a mammal. In an embodiment, the subject is a human.

In an embodiment, the compositions may be used in methods for treating, preventing or diagnosing a disease, disorder or condition. In an embodiment, the method comprises administering to a subject the composition as described herein.

In an embodiment, the compositions may be used in methods for modulating an immune response in a subject. As used herein, the term “modulating” is intended to refer to both immunostimulation (e.g. enhancing an immune response) and immunosuppression (e.g. preventing or decreasing an immune response). Typically, the method would involve one or the other of immunostimulation or immunosuppression, but it is possible that the method could be directed to both. As referred to herein, the “immune response” may either be a cell-mediated (CTL) immune response or an antibody (humoral) immune response.

In some embodiments, the compositions disclosed herein may be used in methods for inducing a cell-mediated immune response to an antigen (e.g. peptide antigens) provided in the composition. In some embodiments, the composition further comprises an agent to enhance the immune response to antigen (e.g. anti-CTLA-4 antibody).

As used herein, the terms “cell-mediated immune response”, “cellular immunity”, “cellular immune response” or “cytotoxic T-lymphocyte (CTL) immune response” (used interchangeably herein) refer to an immune response characterized by the activation of macrophages and natural killer cells, the production of antigen-specific cytotoxic T lymphocytes and/or the release of various cytokines in response to an antigen. Cytotoxic T lymphocytes are a sub-group of T lymphocytes (a type of white blood cell) which are capable of inducing the death of infected somatic or tumor cells; they kill cells that are infected with viruses (or other pathogens), or that are otherwise damaged or dysfunctional. Most cytotoxic T cells express T cell receptors that can recognise a specific peptide antigen bound to Class I MHC molecules. Typically, cytotoxic T cells also express CD8 (i.e. CD8+ T cells), which is attracted to portions of the Class I MHC molecule. This affinity keeps the cytotoxic T cell and the target cell bound closely together during antigen-specific activation. Cellular immunity protects the body by, for example, activating antigen-specific cytotoxic T-lymphocytes (e.g. antigen-specific CD8+ T cells) that are able to lyse body cells displaying epitopes of foreign or mutated antigen on their surface, such as cancer cells displaying tumor-specific antigens (e.g. neoantigens); activating macrophages and natural killer cells, enabling them to destroy intracellular pathogens; and stimulating cells to secrete a variety of cytokines that influence the function of other cells involved in adaptive immune responses and innate immune responses.

Cellular immunity is an important component of the adaptive immune response and following recognition of antigen by cells through their interaction with antigen-presenting cells such as dendritic cells, B lymphocytes and to a lesser extent, macrophages, protect the body by various mechanisms such as:

1. activating antigen-specific cytotoxic T-lymphocytes that are able to induce apoptosis in body cells displaying epitopes of foreign or mutated antigen on their surface, such as cancer cells displaying tumor-specific antigens;

2. activating macrophages and natural killer cells, enabling them to destroy intracellular pathogens; and

3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Since cell-mediated immunity involves the participation of various cell types and is mediated by different mechanisms, several methods could be used to demonstrate the induction of immunity following vaccination. These could be broadly classified into detection of i) specific antigen presenting cells; ii) specific effector cells and their functions and iii) release of soluble mediators such as cytokines.

i) Antigen presenting cells: Dendritic cells and B cells (and to a lesser extent macrophages) are equipped with special immunostimulatory receptors that allow for enhanced activation of T cells, and are termed professional antigen presenting cells (APC). These immunostimulatory molecules (also called co-stimulatory molecules) are up-regulated on these cells following infection or vaccination, during the process of antigen presentation to effector cells such as CD4 and CD8 cytotoxic T cells. Such co-stimulatory molecules (such as CD40, CD80, CD86, MHC class I or MHC class II) can be detected, for example, by using flow cytometry with fluorochrome-conjugated antibodies directed against these molecules along with antibodies that specifically identify APC (such as CD11c for dendritic cells).

ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens. These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perform and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cell-mediated immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flowcytometric measurement of intracellular IFN-γ in these cells.

CD4+“helper” T cells: CD4+ lymphocytes, or helper T cells, are immune response mediators, and play an important role in establishing and maximizing the capabilities of the adaptive immune response. These cells have no cytotoxic or phagocytic activity; and cannot kill infected cells or clear pathogens, but, in essence “manage” the immune response, by directing other cells to perform these tasks. Two types of effector CD4+ T helper cell responses can be induced by a professional APC, designated Th1 and Th2, each designed to eliminate different types of pathogens.

Helper T cells express T cell receptors (TCR) that recognize antigen bound to Class Il MHC molecules. The activation of a naive helper T cell causes it to release cytokines, which influences the activity of many cell types, including the APC that activated it. Helper T cells require a much milder activation stimulus than cytotoxic T cells. Helper T cells can provide extra signals that “help” activate cytotoxic cells. Two types of effector CD4+ T helper cell responses can be induced by a professional APC, designated Th1 and Th2, each designed to eliminate different types of pathogens. The two Th cell populations differ in the pattern of the effector proteins (cytokines) produced. In general, Th1 cells assist the cell-mediated immune response by activation of macrophages and cytotoxic T cells; whereas Th2 cells promote the humoral immune response by stimulation of B cells for conversion into plasma cells and by formation of antibodies. For example, a response regulated by Th1 cells may induce IgG2a and IgG2b in mouse (IgG1 and IgG3 in humans) and favor a cell mediated immune response to an antigen. If the IgG response to an antigen is regulated by Th2 type cells, it may predominantly enhance the production of IgG1 in mouse (IgG2 in humans). The measure of cytokines associated with Th1 or Th2 responses will give a measure of successful vaccination. This can be achieved by specific ELISA designed for Th1-cytokines such as IFN-γ, IL-2, IL-12, TNF-α and others, or Th2-cytokines such as IL-4, IL-5, IL-10 among others.

iii) Measurement of cytokines: released from regional lymph nodes gives a good indication of successful immunization. As a result of antigen presentation and maturation of APC and immune effector cells such as CD4 and CD8 T cells, several cytokines are released by lymph node cells. By culturing these LNC in vitro in the presence of antigen, an antigen-specific immune response can be detected by measuring release if certain important cytokines such as IFN-γ, IL-2, IL-12, TNF-α and GM-CSF. This could be done by ELISA using culture supernatants and recombinant cytokines as standards.

Successful immunization may be determined in a number of ways known to the skilled person including, but not limited to, hemagglutination inhibition (HAIJ) and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g. in the identification of activated or memory lymphocytes. A skilled person may also determine if immunization with a composition as disclosed herein elicited an antibody and/or cell mediated immune response using other known methods.

In an embodiment, the compositions disclosed herein may be used in methods for inducing an antibody immune response to an antigen (e.g. peptide antigens) provided in the composition. In some embodiments, the composition further comprises an agent to enhance the immune response to antigen (e.g. anti-CTLA-4 antibody).

An “antibody immune response” or “humoral immune response” (used interchangeably herein), as opposed to cell-mediated immunity, is mediated by secreted antibodies which are produced in the cells of the B lymphocyte lineage (B cells). Such secreted antibodies bind to antigens, such as for example those on the surfaces of foreign substances, pathogens (e.g. viruses, bacteria, etc.) and/or cancer cells, and flag them for destruction.

As used herein, “humoral immune response” refers to antibody production and may also include, in addition or alternatively, the accessory processes that accompany it, such as for example the generation and/or activation of T-helper 2 (Th2) or T-helper 17 (Th17) cells, cytokine production, isotype switching, affinity maturation and memory cell activation. “Humoral immune response” may also include the effector functions of an antibody, such as for example toxin neutralization, classical complement activation, and promotion of phagocytosis and pathogen elimination. The humoral immune response is often aided by CD4+ Th2 cells and therefore the activation or generation of this cell type may also be indicative of a humoral immune response.

An “antibody” is a protein comprising one or more polypeptides substantially or partially encoded by immunoglobulin genes or fragments of immunoglobulin genes. The recognized immunoglobulin genes include the κ, λ, α, γ, δ, ε and μ constant region genes, as well as myriad immunoglobulin variable region genes. Light chains are classified as either κ or λ. Heavy chains are classified as γ, μ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigenic peptide to T cells, such as T helper CD4 or cytotoxic CD8+ T cells, thus propagating the immune response. B cells, as well as T cells, are part of the adaptive immune response. During an active immune response, induced for example by either vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the TLRs.

Antigen presenting cells, such as dendritic cells and B cells, are drawn to vaccination sites and can interact with antigens and adjuvants contained in a vaccine composition. Typically, the adjuvant stimulates the cells to become activated and the antigen provides the blueprint for the target. Different types of adjuvants may provide different stimulation signals to cells. For example, polyLC (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle 2008; So 2012).

A humoral immune response is one of the common mechanisms for effective infectious disease vaccines (e.g. to protect against viral or bacterial invaders). However, a humoral immune response can also be useful for combating cancer. Whereas a cancer vaccine is typically designed to produce a cell-mediated immune response that can recognize and destroy cancer cells, B cell mediated responses may target cancer cells through other mechanisms which may in some instances cooperate with a cytotoxic T cell for maximum benefit. Examples of B cell mediated (e.g. humoral immune response mediated) anti-tumor responses include, without limitation: 1) Antibodies produced by B cells that bind to surface antigens (e.g. neoantigens) found on tumor cells or other cells that influence tumorigenesis. Such antibodies can, for example. induce killing of target cells through antibody-dependant cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with a tumor or tumor-associated cells to modulate a signaling or cellular pathway that supports cancer; and 4) Antibodies that bind to intracellular targets and mediate anti-tumor activity through a currently unknown mechanism.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Without limitation, other assays that may be used to detect the presence of an antigen-specific antibody include immunological assays (e.g. radioimmunoassay (RIA)), immunoprecipitation assays, and protein blot (e.g. Western blot) assays; and neutralization assays (e.g., neutralization of viral infectivity in an in vitro or in vivo assay).

The compositions disclosed herein may be used in methods for treating or preventing diseases and/or disorders ameliorated by a cell-mediated immune response or a humoral immune response. The compositions and methods disclosed herein may find application in any instance in which it is desired to administer agents (e.g. peptide antigens) to a subject to induce a cell-mediated immune response or a humoral immune response. In an embodiment, the compositions may find application for the delivery of a personalized vaccine, e.g. comprising neoantigens.

In an embodiment, the present disclosure relates to a method comprising administering the composition as described herein to a subject in need thereof. In an embodiment, the method is for the treatment and/or prevention of a disease, disorder or condition in a subject. In an embodiment, the method is for the treatment and/or prevention of an infectious disease or cancer.

In an embodiment, the method is for inducing an antibody immune response and/or cell-mediated immune response to the therapeutic agents (e.g. peptide antigens) in said subject. In an embodiment, such method is for the treatment and/or prevention of an infectious disease or cancer.

Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc.

Treating” may be distinguished from “preventing” in that “treating” typically occurs in a subject who already has a disease or disorder, or is known to have already been exposed to an infectious agent, whereas “preventing” typically occurs in a subject who does not have a disease or disorder, or is not known to have been exposed to an infectious agent. As will be appreciated, there may be overlap in treatment and prevention. For example, it is possible to be “treating” a disease in a subject, while at same time “preventing” symptoms or progression of the disease. Moreover, at least in the context of vaccination, “treating” and “preventing” may overlap in that the treatment of a subject is to induce an immune response that may have the subsequent effect of preventing infection by a pathogen or preventing the underlying disease or symptoms caused by infection with the pathogen. These preventive aspects are encompassed herein by expressions such as “treatment of an infectious disease” or “treatment of cancer”.

In an embodiment, the compositions disclosed herein may be used for treating and/or preventing an infectious disease, such as caused by a viral infection, in a subject in need thereof. The subject may be infected with a virus or may be at risk of developing a viral infection. Viral infections that may be treated and/or prevented by the use or administration of a composition as disclosed herein, without limitation, Cowpoxvirus, Vaccinia virus, Pseudocowpox virus, Human herpesvirus 1, Human herpesvirus 2, Cytomegalovirus, Human adenovirus A-F, Polyomavirus, Human papillomavirus (HPV), Parvovirus, Hepatitis A virus, Hepatitis B virus, Hepatitis C virus, Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus, varicella, severe acute respiratory syndrome coronavirus (SARS-CoV), severe acute respiratory syndrome coronavirus 2 (SARS-CoV-2), and Middle East respiratory syndrome-related coronavirus (MERS-CoV).

In an embodiment, the compositions disclosed herein may be used for treating and/or preventing an infectious disease, such as caused by a non-viral pathogen (such as a bacterium or protozoan) in a subject in need thereof. The subject may be infected with the pathogen or may be at risk of developing an infection by the pathogen. Without limitation, exemplary bacterial pathogens may include Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica. In a particular embodiment, the bacterial infection is Anthrax. Without limitation, exemplary protozoan pathogens may include those of the genus Plasmodium (Plasmodium falciparum, Plasmodium malariae, Plasmodium vivax, Plasmodium ovale or Plasmodium knowlesi), which cause malaria.

In an embodiment, the compositions disclosed herein may be for use in treating and/or preventing cancer in a subject in need thereof. The subject may have cancer or may be at risk of developing cancer.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

Without limitation, cancers that may be capable of being treated and/or prevented by the use or administration of a composition as disclosed herein include carcinoma, adenocarcinoma, lymphoma, leukemia, sarcoma, blastoma, myeloma, and germ cell tumors. Without limitation, particularly suitable embodiments may include glioblastoma, multiple myeloma, ovarian cancer, breast cancer, fallopian tube cancer, prostate cancer or peritoneal cancer. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. In an embodiment, the cancer is one that expresses one or more tumor-specific neoantigens.

In a particular embodiment, the cancer is breast cancer, ovarian cancer, prostate cancer, fallopian tube cancer, peritoneal cancer, glioblastoma or diffuse large B cell lymphoma.

The methods and compositions disclosed herein may be useful for either the treatment or prophylaxis of cancer; for example, a reduction of the severity of cancer (e.g. size of the tumor, aggressiveness and/or invasiveness, malignancy, etc.) or the prevention of cancer recurrences.

In an embodiment, the method for treating and/or preventing cancer first comprises identifying one or more neoantigens or neoepitopes in the patients' tumor cells. The skilled person will understand methods known in the art that can be used to identify the one or more neoantigens (see, for example, Srivastava 2015). As an exemplary embodiment, whole genome/exome sequencing may be used to identify mutated neoantigens that are uniquely present in a tumor of an individual patient. The collection of identified neoantigens can be analyzed to select (e.g. based on algorithms) a specific, optimized subset of neoantigens and/or neoepitopes for use as a personalized cancer vaccine.

Having identified and selected one or more neoantigens, one of skill in the art will appreciate that there are a variety of ways in which to produce such neoantigens either in vitro or in vivo. The neoantigenic peptides may be produced by any method known the art and then may be formulated into a composition or kit as described herein and administered to a subject.

In an embodiment, upon administration to a subject, the composition induces a tumor-specific immune response in the treatment of cancer. By this it is meant that the immune response specifically targets the tumor cells without a significant effect on normal cells of the body which do not express the neoantigen. Further, in an embodiment, the composition may comprise at least one patient-specific neoepitope such that the tumor-specific immune response is patient-specific for the subject or a subset of subjects, i.e. a personalized immunotherapy.

In an embodiment, the compositions disclosed herein may be used for neutralizing toxins, neutralizing viruses, neutralizing bacteria, or neutralizing allergens by providing neutralizing antibodies or by inducing a humoral immune response that generates neutralizing antibodies.

Using the methods as disclosed herein, the composition as disclosed herein may be administered by any suitable route allowing for the at least one hydrophobic phase agent to be targeted to immune cells, lymph nodes, or lymphoid cells in a lymphatic tissue. In an embodiment, the route of administration is sub-cutaneous injection. In an embodiment, the route of administration is intra-muscular injection.

### EMBODIMENTS

Particular embodiments of the disclosure include, without limitation, the following:

1. A composition for delivering at least two agents to a subject comprising:

i) a hydrophobic phase; and

ii) an aqueous phase;

wherein the composition is an emulsion of the hydrophobic phase in the aqueous phase, wherein the hydrophobic phase comprises at least one hydrophobic phase agent, and wherein the aqueous phase comprises at least one aqueous phase agent.

2. The composition of embodiment 1, wherein the ratio of the hydrophobic phase to the aqueous phase is between 70:30 v/v to 50:50 v/v.
3. The composition of embodiment 1 or 2, wherein the hydrophobic phase comprises one or more hydrophobic substances selected from vegetable oil, nut oil, mannide oleate in mineral oil, and sorbitan monooleate in mineral oil.
4. The composition of embodiment 3, wherein the hydrophobic substance comprises mineral oil, mannide oleate in mineral oil, or sorbitan monooleate in mineral oil.
5. The composition of embodiment 4, wherein the hydrophobic substance comprises mannide oleate in mineral oil.
6. The composition of embodiment 4, wherein the hydrophobic substance comprises sorbitan monooleate in mineral oil.
7. The composition of any one of embodiments 3 to 6, wherein the hydrophobic phase comprises a dried preparation of the at least one hydrophobic phase agent reconstituted in the hydrophobic substance.
8. The composition of embodiment 1 or 2, wherein the hydrophobic phase comprises a lipid and cholesterol in mineral oil.
9. The composition of embodiment 8, wherein the lipid is a phospholipid.
10. The composition of embodiment 9, wherein the phospholipid is DOPC.
11. The composition of any one of embodiments 8-10, wherein the hydrophobic phase comprises a dried composition of the lipid, cholesterol, and the at least one hydrophobic phase agent reconstituted in mineral oil.
12. The composition of any one of embodiments 8 to 11, wherein the lipid and cholesterol form lipid vesicle particles in the hydrophobic phase.
13. The composition of embodiment 12, wherein one or more of the at least one hydrophobic phase agent is encapsulated in the lipid vesicle particles.
14. The composition of embodiment 1 or 2, wherein the hydrophobic phase comprises a phospholipid and cholesterol in a hydrophobic substance selected from vegetable oil, nut oil, mineral oil, mannide oleate in mineral oil, and sorbitan monooleate in mineral oil.
15. The composition of embodiment 14, wherein the phospholipid is DOPC.
16. The composition of embodiment 14 or 15, wherein the hydrophobic phase comprises a dried composition of DOPC, cholesterol, and the at least one hydrophobic phase agent reconstituted in mannide oleate in mineral oil or sorbitan monooleate in mineral oil.
17. The composition of embodiment 1 or 2, wherein the hydrophobic phase comprises DOPC and cholesterol in mannide oleate in mineral oil.
18. The composition of embodiment 17, wherein the hydrophobic phase comprises a dried composition of DOPC, cholesterol, and the at least one hydrophobic phase agent reconstituted in mannide oleate in mineral oil.
19. The composition of embodiment 1 or 2, wherein the hydrophobic phase comprises DOPC and cholesterol in sorbitan monooleate in mineral oil.
20. The composition of embodiment 19, wherein the hydrophobic phase comprises a dried composition of DOPC, cholesterol, and the at least one hydrophobic phase agent reconstituted in sorbitan monooleate in mineral oil.
21. The composition of any one of embodiments 15 to 20, wherein the DOPC and cholesterol form lipid vesicle particles in the hydrophobic phase.
22. The composition of embodiment 21, wherein one or more of the at least one hydrophobic phase agent is encapsulated in the lipid vesicle particles.
23. The composition of any one of embodiments 1 to 22, wherein the at least one hydrophobic phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.
24. The composition of embodiment 23, wherein the at least one hydrophobic phase agent is an antigen and an adjuvant.
25. The composition of embodiment 23, wherein the at least one hydrophobic phase agent is an antigen, a T-helper epitope, and an adjuvant.
26. The composition of embodiment 23, wherein the at least one hydrophobic phase agent is peptide antigen of SEQ ID NO: 1, T-helper epitope of SEQ ID NO: 30, and DNA based polyL:C.
27. The composition of embodiment 23, wherein the at least one hydrophobic phase agent is peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C.
28. The composition of embodiment 23, wherein the at least one hydrophobic phase agent is fusion peptide of SEQ ID NO: 34, and DNA based polyL:C.
29. The composition of embodiment 23, wherein the at least one hydrophobic phase agent is peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C.
30. The composition of any one of embodiments 1 to 29, wherein the aqueous phase comprises water, an aqueous solution, or a combination thereof.
31. The composition of embodiment 30, wherein the aqueous phase further comprises an emulsifier.
32. The composition of embodiment 31, wherein the emulsifier is polysorbate 20, polysorbate 80, sorbitan monolaurate, or sorbitan monooleate.
33. The composition of any one of embodiments 30 to 32, wherein the aqueous phase comprises a dried preparation of the at least one aqueous phase agent reconstituted in water, an aqueous solution, or a combination thereof.
34. The composition of any one of embodiments 30 to 33, wherein the aqueous phase further comprises a lipid.
35. The composition of embodiment 34, wherein the lipid is a phospholipid.
36. The composition of embodiment 35, wherein the aqueous phase comprises a dried preparation of the lipid and the at least one aqueous phase agent reconstituted in water, an aqueous solution, or a combination thereof.
37. The composition of any one of embodiments 1 to 36, wherein the at least one aqueous phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.
38. The composition of embodiment 37, wherein the at least one aqueous phase agent is an antibody, a functional equivalent of an antibody, a functional fragment of an antibody, or an antibody mimetic.
39. The composition of embodiment 38, wherein the at one aqueous phase agent is an antibody, a functional equivalent of an antibody, a functional fragment of an antibody, or an antibody mimetic that binds to CTLA-4.
40. The composition of embodiment 39, wherein the at least one aqueous phase agent is an antibody that binds to CTLA-4.
41. A composition for delivering at least two agents to a subject comprising:

i) a hydrophobic phase comprising mannide oleate in mineral oil, DOPC, cholesterol, peptide antigen of SEQ ID NO: 1, T-helper epitope of SEQ ID NO: 30, and DNA based polyL:C; and

ii) an aqueous phase comprising water and/or an aqueous solution, polysorbate 20, and an antibody that binds to CTLA-4; wherein the composition is an emulsion of the hydrophobic phase in the aqueous phase.

42. A composition for delivering at least two agents to a subject comprising:

i) a hydrophobic phase comprising mannide oleate in mineral oil, DOPC, cholesterol, peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C; and

ii) an aqueous phase comprising water and/or an aqueous solution, polysorbate 20, and an antibody that binds to CTLA-4;

wherein the composition is an emulsion of the hydrophobic phase in the aqueous phase.
43. A composition for delivering at least two agents to a subject comprising:

i) a hydrophobic phase comprising mannide oleate in mineral oil, DOPC, cholesterol, fusion peptide of SEQ ID NO: 34, and DNA based polyL:C; and

ii) an aqueous phase comprising water and/or an aqueous solution, polysorbate 20, and an antibody that binds to CTLA-4;

wherein the composition is an emulsion of the hydrophobic phase in the aqueous phase.
44. A composition for delivering at least two agents to a subject comprising:

i) a hydrophobic phase comprising mannide oleate in mineral oil, DOPC, cholesterol, peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C; and

ii) an aqueous phase comprising water and/or an aqueous solution, polysorbate 20, and an antibody that binds to CTLA-4;

wherein the composition is an emulsion of the hydrophobic phase in the aqueous phase.
45. The composition of any one of embodiments 41 to 44, wherein the DOPC and cholesterol form lipid vesicle particles in the hydrophobic phase.
46. The composition of embodiment 45, wherein one or more of the at least one hydrophobic phase agent is encapsulated in the lipid vesicle particles.
47. The composition of any one of embodiments 1 to 46, wherein the emulsion is stable for at least 1 hour.
48. The composition of embodiment 47, wherein the emulsion is stable for at least 4 hours.
49. The composition of any one of embodiments 1 to 48, wherein the composition is for administration to a subject by injection.
50. The composition of embodiment 49, wherein the injection is sub-cutaneous or intra-muscular.
51. A method for making a composition for delivering at least two agents to a subject, said method comprising:

i) providing a hydrophobic phase comprising at least one hydrophobic phase agent;

ii) providing an aqueous phase comprising at least one aqueous phase agent;

iii) mixing the hydrophobic phase and the aqueous phase to produce an emulsion of the hydrophobic phase in the aqueous phase.

52. The method of embodiment 51, wherein the ratio of the hydrophobic phase to the aqueous phase is between 70:30 v/v to 50:50 v/v.
53. The method of embodiment 51 or 52, wherein the hydrophobic phase comprises one or more hydrophobic substances selected from vegetable oil, nut oil, mineral oil, mannide oleate in mineral oil, and sorbitan monooleate in mineral oil.
54. The method of embodiment 53, wherein the hydrophobic substance comprises mineral oil, mannide oleate in mineral oil, or sorbitan monooleate in mineral oil.
55. The method of embodiment 54, wherein the hydrophobic substance comprises mannide oleate in mineral oil.
56. The method of embodiment 54, wherein the hydrophobic substance comprises sorbitan monooleate in mineral oil.
57. The method of embodiment 55 or 56, wherein the hydrophobic phase is produced by reconstituting a dried preparation of the at least one hydrophobic phase agent in the hydrophobic substance.
58. The method of embodiment 51 or 52, wherein the hydrophobic phase comprises a lipid and cholesterol in mineral oil.
59. The method of embodiment 58, wherein the lipid is a phospholipid.
60. The method of embodiment 59, wherein the phospholipid is DOPC.
61. The method of embodiment 58 or 59, wherein the hydrophobic phase is produced by reconstituting a dried composition of the lipid, cholesterol, and the at least one hydrophobic phase agent in mineral oil.
62. The method of any one of embodiments 58 to 61, wherein the lipid and cholesterol form lipid vesicle particles in the hydrophobic phase.
63. The method of embodiment 62, wherein one or more of the at least one hydrophobic phase agent is encapsulated in the lipid vesicle particles.
64. The method of embodiment 51 or 52, wherein the hydrophobic phase comprises a phospholipid and cholesterol in a hydrophobic substance selected from vegetable oil, nut oil, mineral oil, mannide oleate in mineral oil, and sorbitan monooleate in mineral oil.
65. The method of embodiment 64, wherein the phospholipid is DOPC.
66. The method of embodiment 64 or 65, wherein the hydrophobic phase is produced by reconstituting a dried composition of DOPC, cholesterol, and the at least one hydrophobic phase agent in mannide oleate in mineral oil or sorbitan monooleate in mineral oil.
67. The method of embodiment 51 or 52, wherein the hydrophobic phase comprises DOPC and cholesterol in mannide oleate in mineral oil.
68. The method of embodiment 67, wherein the hydrophobic phase is produced by reconstituting a dried composition of DOPC, cholesterol, and the at least one hydrophobic phase agent in mannide oleate in mineral oil.
69. The method of embodiment 51 or 52, wherein the hydrophobic phase comprises DOPC and cholesterol in sorbitan monooleate in mineral oil.
70. The method of embodiment 69, wherein the hydrophobic phase is produced by reconstituting a dried composition of DOPC, cholesterol, and the at least one hydrophobic phase agent in sorbitan monooleate in mineral oil.
71. The method of any one of embodiments 65 to 70, wherein the DOPC and cholesterol form lipid vesicle particles in the hydrophobic phase.
72. The method of embodiment 71, wherein one or more of the at least one hydrophobic phase agent is encapsulated in the lipid vesicle particles.
73. The method of any one of embodiments 51 to 72, wherein the at least one hydrophobic phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.
74. The method of embodiment 73, wherein the at least one hydrophobic phase agent is an antigen and an adjuvant.
75. The method of embodiment 73, wherein the at least one hydrophobic phase agent is an antigen, a T-helper epitope, and an adjuvant.
76. The method of embodiment 73, wherein the at least one hydrophobic phase agent is peptide antigen of SEQ ID NO: 1, T-helper epitope of SEQ ID NO: 30, and DNA based polyL:C.
77. The method of embodiment 73, wherein the at least one hydrophobic phase agent is peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C.
78. The method of embodiment 73, wherein the at least one hydrophobic phase agent is fusion peptide of SEQ ID NO: 34, and DNA based polyL:C.
79. The method of embodiment 73, wherein the at least one hydrophobic phase agent is peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C.
80. The method of any one of embodiments 51 to 79, wherein the aqueous phase comprises water, an aqueous solution, or a combination thereof.
81. The method of embodiment 80, wherein the aqueous phase further comprises an emulsifier.
82. The method of embodiment 81, wherein the emulsifier is polysorbate 20, polysorbate 80, sorbitan monolaurate, or sorbitan monooleate.
83. The method of any one of embodiments 80 to 82, wherein the aqueous phase is produced by reconstituting a dried preparation of the at least one aqueous phase agent in water, an aqueous solution, or a combination thereof.
84. The method of any one of embodiments 80 to 82, wherein the aqueous phase further comprises a lipid.
85. The method of embodiment 84, wherein the lipid is a phospholipid.
86. The method of embodiment 84 or 85, wherein the aqueous phase is produced by reconstituting a dried composition of the lipid and the at least one aqueous phase agent in water, an aqueous solution, or a combination thereof.
87. The method of any one of embodiments 51 to 86, wherein the at least one aqueous phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.
88. The method of embodiment 87, wherein the at one aqueous phase agent is an antibody, a functional equivalent of an antibody, a functional fragment of an antibody, or an antibody mimetic.
89. The method of embodiment 88, wherein the at one aqueous phase agent is an antibody, a functional equivalent of an antibody, a functional fragment of an antibody, or an antibody mimetic that binds to CTLA-4.
90. The method of embodiment 89, wherein the at least one aqueous phase agent is an antibody that binds to CTLA-4.
91. The method of embodiment 51 or 52, wherein:

i) the hydrophobic phase comprises mannide oleate in mineral oil, DOPC, cholesterol, peptide antigen of SEQ ID NO: 1, T-helper epitope of SEQ ID NO: 30, and DNA based polyL:C; and

ii) the aqueous phase comprises water and/or an aqueous solution, polysorbate 20, and an antibody that binds to CTLA-4.

92. The method of embodiment 91, wherein the hydrophobic phase is produced by reconstituting a dried composition of DOPC, cholesterol, peptide antigen of SEQ ID NO: 1, T-helper epitope of SEQ ID NO: 30, and DNA based polyL:C in mannide oleate in mineral oil.
93. The method of embodiment 51 or 52, wherein:

i) the hydrophobic phase comprises mannide oleate in mineral oil, DOPC, cholesterol, peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C; and

ii) the aqueous phase comprises water and/or an aqueous solution, polysorbate 20, and an antibody that binds to CTLA-4.

94. The method of embodiment 93, wherein the hydrophobic phase is produced by reconstituting a dried composition of DOPC, cholesterol, peptide antigen of SEQ ID NO: 18, peptide antigen of SEQ ID NO: 20, peptide antigen of SEQ ID NO: 22, peptide antigen of SEQ ID NO: 23, peptide antigen of SEQ ID NO: 24, T-helper epitope of SEQ ID NO: 28, and DNA based polyL:C in mannide oleate in mineral oil.
95. The method of embodiment 51 or 52, wherein:

i) the hydrophobic phase comprises mannide oleate in mineral oil, DOPC, cholesterol, fusion peptide of SEQ ID NO: 34, and DNA based polyL:C; and

ii) the aqueous phase comprises water and/or an aqueous solution, polysorbate 20, and an antibody that binds to CTLA-4.

96. The method of embodiment 95, wherein the hydrophobic phase is produced by reconstituting a dried composition of DOPC, cholesterol, fusion peptide of SEQ ID NO: 34, and DNA based polyL:C in mannide oleate in mineral oil.
97. The method of embodiment 51 or 52, wherein:

i) the hydrophobic phase comprises mannide oleate in mineral oil, DOPC, cholesterol, peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C; and

ii) the aqueous phase comprises water and/or an aqueous solution, polysorbate 20, and an antibody that binds to CTLA-4.

98. The method of embodiment 97, wherein the hydrophobic phase is produced by reconstituting a dried composition of DOPC, cholesterol, peptide antigen of SEQ ID NO: 35, peptide antigen of SEQ ID NO: 36, peptide antigen of SEQ ID NO: 37, peptide antigen of SEQ ID NO: 38, peptide antigen of SEQ ID NO: 20, peptide antigen of SEQ ID NO: 23, T-helper epitope of SEQ ID NO: 28, and DNA based polyL:C in mannide oleate in mineral oil.
99. The method of any one of embodiments 93 to 98, wherein the DOPC and cholesterol form lipid vesicle particles in the hydrophobic phase.
100. The method of embodiment 99, wherein one or more of the at least one hydrophobic phase agent is encapsulated in the lipid vesicle particles.
101. The method of any one of embodiments 91 to 100, wherein the aqueous phase is produced by reconstituting a dried preparation of the antibody that binds to CTLA-4 in water and/or an aqueous solution.
102. The method of any one of embodiments 51 to 101, wherein the emulsion is stable for at least 1 hour.
103. The method of embodiment 102, wherein the emulsion is stable for at least 4 hours.
104. The method of any one of embodiments 51 to 103, wherein the hydrophobic phase and the aqueous phase are mixed by placing the hydrophobic phase and the aqueous phase in a vessel and agitating the vessel with a vortex mixer.
105. The method of any one of embodiments 51 to 103, wherein the hydrophobic phase and the aqueous phase are mixed by aspirating the hydrophobic phase into a first syringe, aspirating the aqueous phase into a second syringe, connecting the first syringe and the second syringe to a connector, and applying alternating pressure to the first and second syringes to repeatedly pass the phases through the connector.
106. A composition produced by the method of any one of embodiments 51 to 105.
107. A method for delivering at least two agents to a subject, said method comprising administering to the subject the composition of any one of embodiments 1 to 50.
108. A method for inducing an immune response in a subject, comprising administering to the subject the composition of any one of embodiments 1 to 50.
109. The method of embodiment 108, wherein the immune response is an antibody response and/or a cell-mediated response.
110. A method for treating, preventing or diagnosing a disease, disorder or condition in a subject, comprising administering to the subject the composition of any one of embodiments 1 to 50.
111. A method for modulating an immune response in a subject, comprising administering to the subject the composition of any one of embodiments 1 to 50.
112. A method for treating or preventing diseases and/or disorders ameliorated by a cell-mediated immune response or a humoral immune response in a subject, comprising administering to the subject the composition of any one of embodiments 1 to 50.
113. A method for treating and/or preventing an infectious disease caused by a virus, bacteria, or protozoa in a subject, comprising administering to the subject the composition of any one of embodiments 1 to 50.
114. A method for treating and/or preventing cancer in a subject, comprising administering to the subject the composition of any one of embodiments 1 to 50.
115. The method of embodiment 114, wherein the cancer is carcinoma, adenocarcinoma, lymphoma, leukemia, sarcoma, blastoma, myeloma, ovarian cancer, breast cancer, fallopian tube cancer, prostate cancer or peritoneal cancer.
116. A method for neutralizing a toxin, virus, bacterium, or allergen with an antibody in a subject, said method comprising administering to the subject the composition of any one of embodiments 1 to 50.
117. The method of any one of embodiments 107 to 116 wherein the composition is administered to the subject by injection.
118. The method of embodiment 117, wherein the injection is sub-cutaneous or intra-muscular.
119. A kit comprising:

a) a first container comprising a dried preparation of at least one hydrophobic phase agent;

b) a second container comprising one or more hydrophobic substances; and

c) a third container comprising an aqueous solution comprising at least one aqueous phase agent.

120. A kit comprising:

a) a first container comprising a dried preparation of at least one hydrophobic phase agent;

b) a second container comprising one or more hydrophobic substances;

c) a third container comprising a dried preparation of at least one aqueous phase agent; and

d) a fourth container comprising water, an aqueous solution, or a combination thereof.

121. The kit of embodiments 119 or 120, wherein the kit further comprises at least two syringes.
122. The kit of any one of embodiments 119 to 121, wherein the kit further comprises a connector for connecting the at least two syringes.
123. The kit of any one of embodiments 119 to 122, wherein the dried preparation of at least one hydrophobic phase agent further comprises DOPC and cholesterol.
124. The kit of any one of embodiments 119 to 123, wherein the one or more hydrophobic substances comprises vegetable oil, nut oil, or mineral oil.
125. The kit of embodiment 124, wherein the hydrophobic substance comprises mannide oleate in mineral oil.
126. The kit of embodiment 124, wherein the hydrophobic substance comprises sorbitan monooleate in mineral oil.
127. The kit of any one of embodiments 119 to 126, wherein the aqueous solution comprises phosphate buffered saline.
128. Use of the composition of any one of embodiments 1 to 50 for delivering at least two agents to a subject.
129. Use of the composition of any one of embodiments 1 to 50 for inducing an immune response in a subject.
130. The use of embodiment 129, wherein the immune response is an antibody response and/or a cell-mediated response.
131. Use of the composition of any one of embodiments 1 to 50 for treating, preventing or diagnosing a disease, disorder or condition in a subject.
132. Use of the composition of any one of embodiments 1 to 50 for modulating an immune response in a subject.
133. Use of the composition of any one of embodiments 1 to 50 for treating or preventing diseases and/or disorders ameliorated by a cell-mediated immune response or a humoral immune response in a subject.
134. Use of the composition of any one of embodiments 1 to 50 for treating and/or preventing an infectious disease caused by a virus, bacteria, or protozoa in a subject.
135. Use of the composition of any one of embodiments 1 to 50 for treating and/or preventing cancer in a subject.
136. The use of embodiment 135, wherein the cancer is carcinoma, adenocarcinoma, lymphoma, leukemia, sarcoma, blastoma, myeloma, ovarian cancer, breast cancer, fallopian tube cancer, prostate cancer or peritoneal cancer.
137. Use of the composition of any one of embodiments 1 to 50 for neutralizing a toxin, virus, bacterium, or allergen with an antibody in a subject.
138. The use of any one of embodiments 128 to 137 wherein the composition is for administration to the subject by injection.
139. The use of embodiment 138, wherein the injection is sub-cutaneous or intra-muscular.

### EXAMPLES

The invention will now be described by way of non-limiting examples having regard to the appended drawings.

### Example 1

Preparation of O/W emulsion using mineral oil and water with surfactant.

0.7 mL of mineral oil (hydrophobic phase) was taken up in a syringe, while 0.3 mL of water with surfactant (aqueous phase) was taken up in another syringe. The two syringes were connected using a Vygon™ connector three-way stopcock, and the phases were mixed by passing them 120 times between the syringes through the stopcock to form an emulsion using a 70:30 hydrophobic:aqueous ratio, beginning by passing the hydrophobic phase into the aqueous phase. Four emulsions were prepared using water containing either 0.5% Tween™ 20, 0.25% Tween™ 80, 0.5% Tween™ 80, or 0.5% PEG 400 by weight as the aqueous phase.

To evaluate emulsion stability, the emulsion was observed for phase separation or cracking for 4 hours. The emulsions were also subjected to drop tests and cobalt chloride paper test to identify whether the mixtures formed oil-in-water (O/W) or water-in-oil (W/O) emulsions. A water drop test consists of filling a 250 mL beaker with −200 mL of distilled water and placing a drop (˜3 mg) of the prepared emulsion on top of the water without stirring. Dispersion of the drop into the water indicates that the emulsion is O/W. If the drop floats on the water and the water remains clear after gentle hand stirring then the emulsion is W/O. An oil drop test consists of filling a 5 mL glass vial with ˜4 mL of mineral oil and placing a drop (˜3 mg) of the prepared emulsion on top of the oil without stirring. If the drop does not disperse in the oil then the emulsion is O/W. Dispersion of the drop into the oil indicates that the emulsion is W/O. Cobalt chloride paper test: When a drop of emulsion is placed on cobalt chloride filter paper strip, it turns the paper strip from blue to pink, indicating that the formed emulsion is O/W.

The results are shown below in Table 1. The emulsions formed using 0.5% Tween™ 20, 0.25% Tween™ 80, or 0.5% Tween™ 80 were stable for more than 4 hours, whereas the emulsion formed using PEG 400 separated within 30 minutes. Water drop test showed that all emulsions were O/W emulsions.

TABLE 1

| Emulsion Formulation | Emulsion Stability | Emulsion Type |
|---|---|---|
| 30% (0.5% Tween 20 | Stable for more | Oil-in-Water |
| in Sterile water H2O): | than 4 hours | |
| 70% Mineral oil | | |
| 30% (0.25% Tween 80 | Stable for more | Oil-in-Water |
| in Sterile water H2O): | than 4 hours | |
| 70% Mineral oil | | |
| 30% (0.5% Tween 80 | Stable for more | Oil-in-Water |
| in Sterile water H2O): | than 4 hours | |
| 70% Mineral oil | | |
| 30% (0.5% PEG400 | Separated in less | Oil-in-Water |
| in Sterile water H2O): | than 0.5 hour | |
| 70% Mineral oil | | |

These results indicate that a stable O/W emulsion can be formed using mineral oil and an aqueous phase containing surfactants.

### Example 2

Preparation of O/W emulsion using DPX reconstituted in MS80 oil and mixed with water containing surfactant.

A hydrophobic phase was prepared by reconstituting a freeze-dried composition (DPX) in MS80 oil. DPX is a composition comprising amphiphilic lipids (DOPC), cholesterol, peptides, and adjuvant. DPX-R9F (0.1 mg/mL HPV16E7₄₉₋₅₇ peptide antigen [R9F; SEQ ID NO: 1], 0.1 mg/mL universal T helper epitope derived from tetanus toxin₉₄₇₋₉₆₇ [F21E; SEQ ID NO: 30], 0.4 mg/mL poly dIdC polynucleotide) was reconstituted in 0.7 mL MS80 oil to form a hydrophobic phase. The hydrophobic phase was then mixed with an aqueous phase containing either 0.5% Tween™ 20, 0.25% Tween™ 80, 0.5% Tween™ 80, or 0.5% PEG 400 by weight at 90:10, 80:20, and 70:30 hydrophobic:aqueous ratios by volume and mixed by vortex for 2 minutes to form an emulsion. To evaluate emulsion stability, the emulsion was observed for phase separation or cracking for 4 hours. The emulsions were also subjected to drop tests to identify whether the mixtures formed O/W or W/O emulsions.

The results are shown below in Table 2. Using DPX-R9F/MS80 oil as the hydrophobic phase, only a 70:30 hydrophobic:aqueous ratio produced a stable emulsion, with the other ratios separating either immediately or within 30 minutes. However, a water drop test indicated that the emulsion was W/O. Without being bound by theory, it was possible that the presence of Span™ 80 surfactant in the MS80 oil can invert or inhibit the formation of O/W emulsions due to the low HLB value of Span™ 80.

TABLE 2

| Emulsion Formulation | Emulsion Stability | Emulsion Type |
|---|---|---|
| 30% (0.5% Tween 20 in | Stable for more | Water-in-Oil |
| Sterile water H2O): | than 4 hours | |
| 70% DPX-R9F in MS80 | | |
| 30% (0.25% Tween 80 in | Stable for more | Water-in-Oil |
| Sterile water H2O): | than 4 hours | |
| 70% DPX-R9F in MS80 | | |
| 30% (0.5% Tween 80 in | Stable for more | Water-in-Oil |
| Sterile water H2O): | than 4 hours | |
| 70% DPX-R9F in MS80 | | |
| 30% (0.5% PEG400 in | Stable for more | Water-in-Oil |
| Sterile water H2O): | than 4 hours | |
| 70% DPX-R9F in MS80 | | |

Mixtures using a 50:50 hydrophobic:aqueous ratio where prepared. DPX-Survivac freeze-dried composition (1 mg/mL 5 survivin peptide antigens [SEQ ID NOs: 18, 20, 22, 23, and 24], 0.5 mg/mL A16L tetanus toxin helper peptide [SEQ ID NO: 28], 0.4 mg/mL poly dIdC polynucleotide) was reconstituted in MS80 oil to form a hydrophobic phase and mixed with an aqueous phase containing either 0.5% Tween™ 20 or 0.5% Tween™ 80 by weight at a 50:50 hydrophobic:aqueous ratio by repeated passing through a Vygon™ connector. The results are shown below in Table 3. The mixtures formed emulsions that were stable for more than 4 hours. A water drop test indicated that the emulsions were O/W that dispersed quickly in water.

TABLE 3

| | | | Dispersion in |
|---|---|---|---|
| Emulsion Formulation | Emulsion Stability | Emulsion Type | Water |
| 50% (0.5% Tween 20 in | Stable for more than 4 | Oil-in-Water | Fast dispersion |
| Sterile water H2O): 50% | hours | | in water |
| DPX-Survivac in MS80 | | | |
| 50% (0.5% Tween 80 in | Stable for more than 4 | Oil-in-Water | Fast dispersion |
| Sterile water H2O): 50% | hours | | in water |
| DPX-Survivac in MS80 | | | |

These results indicate that a stable O/W emulsion can be formed using an amphiphilic lipid-containing DPX composition in oil containing surfactant and an aqueous phase containing surfactant.

### Example 3

Preparation of O/W emulsion using DPX reconstituted in mineral oil and mixed with water containing surfactant.

A hydrophobic phase was prepared by reconstituting DPX-R9F freeze-dried composition with 0.7 mL of mineral oil. The hydrophobic phase was then mixed with 0.3 mL of an aqueous phase containing either 0.5% Tween™ 20, 0.25% Tween™ 80, 0.5% Tween™ 80, or 0.5% PEG 400 by weight and mixed by vortex mixing for 2 minutes to form an emulsion with a 70:30 hydrophobic:aqueous ratio.

The results are shown below in Table 4. A water drop test indicated that the emulsions were O/W. The emulsions containing Tween™ 20 or Tween™ 80 were stable for more than 4 hours, while the emulsion containing PEG 400 separated within 1 hour.

TABLE 4

| | | | Dispersion in |
|---|---|---|---|
| Emulsion Formulation | Emulsion Stability | Emulsion Type | Water |
| 30% (0.5% Tween 20 in | Stable for more than 4 | Oil-in-Water | Slow dispersion |
| Sterile water H2O): 70% | hours | | in water |
| DPX-R9F in Mineral oil | | | |
| 30% (0.25% Tween 80 in | Stable for more than 4 | Oil-in-Water | Slow dispersion |
| Sterile water H2O): 70% | hours | | in water |
| DPX-R9F in Mineral oil | | | |
| 30% (0.5% Tween 80 in | Stable for more than 4 | Oil-in-Water | Slow dispersion |
| Sterile water H2O): 70% | hours | | in water |
| DPX-R9F in Mineral oil | | | |
| 30% (0.5% PEG400 in | Phase separation in less | Oil-in-Water | Fast dispersion |
| Sterile water H2O): 70% | than 1 hour | | in water |
| DPX-R9F in Mineral oil | | | |

These results indicate that a stable O/W emulsion can be formed using an amphiphilic lipid-containing DPX composition in oil and an aqueous phase containing surfactant.

### Example 4

Preparation of O/W emulsions using DPX reconstituted in mineral oil mixed with water containing surfactant, using different mixing methods.

A hydrophobic phase was prepared by reconstituting DPX-Survivac freeze-dried composition with 0.7 mL of mineral oil and mixed with 0.3 mL of an aqueous phase containing either 0.5% Tween™ 20, 0.25% Tween™ 80, 0.5% Tween™ 80, or 0.5% PEG 400 by weight. The phases were mixed either by vortex mixing for 2 minutes or by repeated passing of the phases between syringes through a Vygon™ connector.

The results using emulsification by vortex mixing are shown below in Table 5. The emulsions containing Tween™ 20 or Tween™ 80 were stable for more than 4 hours, while the emulsion containing PEG 400 separated within 1 hour. A water drop test indicated that all emulsions were O/W. The results using emulsification by passing through a connector are shown below in Table 6. As with the vortex mixing method, all of the emulsions were O/W and all were stable for more than 4 hours except for the emulsion containing PEG 400 which separated within 1 hour. The O/W emulsions formed by passing through a connector dispersed faster in water than the emulsions formed by vortex mixing.

TABLE 5

| | | | Dispersion in |
|---|---|---|---|
| Emulsion Formulation | Emulsion Stability | Emulsion Type | Water |
| 30% (0.5% Tween 20 in | Stable for more than 4 | Oil-in-Water | Slow dispersion |
| Sterile water H2O): 70% | hours | | in water |
| DPX- Survivac in Mineral | | | |
| oil | | | |
| 30% (0.25% Tween 80 in | Stable for more than 4 | Oil-in-Water | Slow dispersion |
| Sterile water H2O): 70% | hours | | in water |
| DPX- Survivac in Mineral | | | |
| oil | | | |
| 30% (0.5% Tween 80 in | Stable for more than 4 | Oil-in-Water | Slow dispersion |
| Sterile water H2O): 70% | hours | | in water |
| DPX- Survivac in Mineral | | | |
| oil | | | |
| 30% (0.5% PEG400 in | Phase separation in | Oil-in-Water | Slow dispersion |
| Sterile water H2O): 70% | less than 1 hour | | in water |
| DPX- Survivac in Mineral | | | |
| oil | | | |

TABLE 6

| | | | Dispersion in |
|---|---|---|---|
| Emulsion Formulation | Emulsion Stability | Emulsion Type | Water |
| 50% (0.5% Tween 20 in | Stable for more than 4 | Oil-in-Water | Fast dispersion |
| Sterile water H2O): 50% | hours | | in water |
| DPX- Survivac in Mineral | | | |
| oil | | | |
| 30% (0.25% Tween 80 in | Stable for more than 4 | Oil-in-Water | Fast dispersion |
| Sterile water H2O): 70% | hours | | in water |
| DPX- Survivac in Mineral | | | |
| oil | | | |
| 30% (0.5% Tween 80 in | Stable for more than 4 | Oil-in-Water | Fast dispersion |
| Sterile water H2O): 70% | hours | | in water |
| DPX- Survivac in Mineral | | | |
| oil | | | |
| 30% (0.5% PEG400 in | Phase separation in less | Oil-in-Water | Fast dispersion |
| Sterile water H2O): 70% | than 1 hour | | in water |
| DPX- Survivac in Mineral | | | |
| oil | | | |

### Example 5

Preparation of O/W emulsions using DPX reconstituted in Montanide™ ISA51 VG oil mixed with water containing surfactant, using different surfactants, mixing methods, and ratios.

A hydrophobic phase was prepared by reconstituting DPX-Survivac freeze-dried composition with Montanide™ ISA51 VG oil and mixed with an aqueous phase containing either 0.5% Tween™ 20 or 0.5% Tween™ 80 by weight. The phases were mixed either in a 70:30 or a 50:50 hydrophobic:aqueous ratio, and mixed either by vortex mixing or by repeated passing between syringes through a Vygon™ connector to form an emulsion.

The results are shown below in Table 7 (mixing by passage) and Table 8 (mixing by vortex). Using either method of mixing, the 70:30 hydrophobic:aqueous ratio emulsions were more viscous than the 50:50 ratio emulsions. All emulsions were stable for more than 4 hours and were O/W as indicated by water drop tests, oil drop tests, and cobalt paper test. The emulsions formed by vortex mixing using Tween™ 20 sank in oil during the oil drop test, indicating that they were higher density than the emulsions formed by passing through a connector. The emulsion formed by mixing by passage using a 70:30 ratio with Tween 80™ dispersed more slowly in water than a similar emulsion using Tween 20™.

TABLE 7

| Emulsion | | Emulsion | Drop Test | Drop Test | Cobalt |
|---|---|---|---|---|---|
| Formulation | pH | Stability | (water) | (oil) | Paper test |
| 0.5% Tween 20 in | 5.88 | Stable for | Disperses | Floats (oil-in- | Pink |
| S•H₂O + DPX- | | more than 4 | (oil-in-water) | water) | |
| Survivac in ISA51 | | hours | | | |
| Ratio: 0.5 mL: 0.5 mL | | | | | |
| 0.5% Tween 20 in | 6.22 | Stable for | Disperses | Floats (oil-in- | Pink |
| S•H₂O + DPX- | | more than 4 | (oil-in-water) | water) | |
| Survivac in ISA51 | | hours | | | |
| Ratio: 0.3 mL: 0.7 mL | | | | | |
| 0.5% Tween 80 in | 6.75 | Stable for | Disperses | Floats (oil-in- | Pink |
| S•H₂O + DPX- | | more than 4 | (oil-in-water) | water) | |
| Survivac in ISA51 | | hours | | | |
| Ratio: 0.3 mL: 0.7 mL | | | | | |

TABLE 8

| Emulsion | | Emulsion | Drop Test | Drop Test | Cobalt |
|---|---|---|---|---|---|
| Formulation | pH | Stability | (water) | (oil) | Paper test |
| 0.5% Tween 20 in | 6.05 | Stable for | Disperses | Sinks (oil-in- | Pink |
| S•H₂O + DPX- | | more than 4 | (oil-in-water) | water) | |
| Survivac in ISA51 | | hours | | | |
| Ratio: 0.5 mL: 0.5 mL | | | | | |
| 0.5% Tween 20 in | 7.26 | Stable for | Disperses | Sinks (oil-in- | Pink |
| S•H₂O + DPX- | | more than 4 | (oil-in-water) | water) | |
| Survivac in ISA51 | | hours | | | |
| Ratio: 0.3 mL: 0.7 mL | | | | | |
| 0.5% Tween 80 in | 6.68 | Stable for | Disperses | Floats (oil-in- | Pink |
| S•H₂O + DPX- | | more than 4 | (oil-in-water) | water) | |
| Survivac in ISA51 | | hours | | | |
| Ratio: 0.3 mL: 0.7 mL | | | | | |

These results indicate that a stable O/W emulsion can be formed using an amphiphilic lipid-containing DPX composition in Montanide™ ISA51 oil and an aqueous phase containing surfactant.

### Example 6

Preparation of O/W emulsion using DPX reconstituted in mineral oil or MS80 oil with water containing anti-CTLA-4 antibody or albumin.

A hydrophobic phase was prepared by reconstituting DPX-FP (0.2 mg/mL FP antigen [SEQ ID NO: 34], 0.4 mg/mL poly dIdC polynucleotide) with 0.7 mL of either mineral oil or MS80 oil. The hydrophobic phase was mixed with 0.3 mL of an aqueous phase containing 0.5% Tween™ 20 and 6.7 mg/mL anti-CTLA-4 antibody and mixed by vortex mixing for 2 minutes to form an emulsion with a 70:30 hydrophobic:aqueous ratio. The final concentration of anti-CTLA-4 antibody in the emulsion was 2.0 mg/mL. The results are shown below in Table 9. The MS80 oil emulsion was W/O and separated in less than 1 hour. This is consistent with the results of Example 2 showing that a lower hydrophobic:aqueous ratio (e.g. 50:50) is necessary to form an O/W emulsion with MS80 oil in the hydrophobic phase. The mineral oil emulsion was O/W and remained stable for over 4 hours.

TABLE 9

| Emulsion Formulation | Emulsion Stability | Emulsion Type |
|---|---|---|
| 30% (0.5% Tween 20 containing anti- | Phase separation in less than 1 | Water-in-Oil |
| CTLA4): 70% (DPX-FP in MS80) | hour | |
| 30% (0.5% Tween 20 containing anti- | Stable for more than 4 hours | Oil-in-Water |
| CTLA4): 70% (DPX-FP in Mineral | | |
| oil) | | |

A different O/W emulsion was produced by preparing a hydrophobic phase of DPX-FP in Montanide™ ISA51 VG oil. An aqueous phase was prepared by mixing a solution of 8.47 mg/mL albumin in PBS with Tween™ 20 in sterile water to obtain an aqueous phase of 0.5% Tween™ 20 by weight and 6.7 mg/mL albumin. The phases were then mixed either by repeated passing between syringes through a Vygon™ connector or by vortex mixing to form an emulsion. The results are shown below in Table 10. Both emulsions were O/W. The emulsion formed by passing through a connector was stable for more than 4 hours, while the emulsion formed by vortex mixing was stable for 2 hours.

TABLE

| Emulsion | | Emulsion | Drop Test | Drop Test | Cobalt |
|---|---|---|---|---|---|
| Formulation | pH | Stability | (water) | (oil) | Paper test |
| 6.7 mg/mL Albumin | 7.40 | Stable for | Disperses | Sinks (oil-in- | Pink |
| and 0.5% Tween 20 | | more than 4 | (oil-in-water) | water) | |
| in PBS and S•H₂O + | | hours | | | |
| DPX-FP in ISA51 | | | | | |
| Ratio: 0.3 mL: 0.7 mL | | | | | |
| Mixing by Passage | | | | | |
| 6.7 mg/mL | 6.81 | Stable for 2 | Disperses | Sinks (oil-in- | Pink |
| Albumin and 0.5% | | hours | (oil-in-water) | water) | |
| Tween 20 in PBS | | | | | |
| and S•H₂O + DPX- | | | | | |
| FP in ISA51 | | | | | |
| Ratio: 0.3 mL: 0.7 mL | | | | | |
| Mixing by Vortex | | | | | |

These results indicate that a stable O/W emulsion can be formed using an amphiphilic lipid-containing DPX composition in Montanide™ ISA51 oil and an aqueous phase containing surfactant and a water-soluble molecule.

### Example 7

Treatment of tumor-challenged mice with an O/W emulsion to simultaneously deliver DPX-FP and anti-CTLA-4 antibody.

The efficacy of an O/W emulsion to simultaneously deliver DPX-FP and anti-CTLA-4 antibody in controlling tumor growth was assessed and compared to control treatments. It was evaluated whether anti-CTLA-4 antibody, delivered in an O/W emulsion with DPX-FP can control tumor growth in the same way as systemic delivery of anti-CTLA-4 via intraperitoneal (i.p.) injection or by delivery of anti-CTLA-4 antibody in a hydrophobic carrier. The study was conducted in C3 tumor-bearing mice and included combination therapy with metronomic cyclophosphamide (mCPA).

An O/W emulsion according to the invention was prepared using DPX-FP freeze-dried composition. DPX-FP was prepared by adding FP (NeoMPS; SEQ ID NO: 34) and DNA based polyL:C polynucleotide adjuvant stock (Biospring) to a lipid-mixture solution, mixing well and freeze-drying. A lipid-mixture (132 mg/mL) containing DOPC and cholesterol in a 10:1 ratio (w:w) (Lipoid GmBH, Germany) was dissolved in 40% tertiary-butanol by shaking well at 300 RPM at room temperature for 1 hour or until dissolved. Next, FP stock (10 mg/mL) was prepared in DMSO and DNA based polyL:C polynucleotide adjuvant stock (10 mg/mL) was prepared in sterile water. To a 0.8 mL aliquot of lipid-mixture solution, 16 μL of FP stock was added to obtain 0.1 mg/mL final fill concentration, shaken well at 300 RPM for 5 minutes. To the formed FP-lipid-mixture solution, 32 μL of DNA based polyL:C polynucleotide adjuvant stock was added to obtain 0.2 mg/mL final fill concentration, shaken well at 300 RPM for 5 minutes. Q.S to 1.6 mL with 40% tertiary-butanol and freeze-dried.

An O/W emulsion according to the invention was prepared using a 70:30 hydrophobic:aqueous ratio. A hydrophobic phase was prepared by reconstituting three vials of DPX-FP (IMV, DPX20-181109-1) by adding 1.0 mL of ISA 51 oil (SEPPIC) to each vial. Freeze-dried DPX-FP was soaked in the oil for 5 mins and vortexed well for 2 mins to form a clear solution. An aqueous phase was prepared by first adding 409.23 mg of Tween™ 20 (Sigma Aldrich, SLBZ5913) to a 15-mL falcon tube, then adding 9.815 g of sterile water (Baxter, W7F0520) to the tube and mixing by vortex to form a 4% Tween™ 20 stock solution. Then, 0.265 mL of anti-CTLA-4 (Biocell, 702418A2B, 7.54 mg/mL in PBS, pH 7.0) was added to a 1.5-mL Eppendorf tube along with 35 μL of 4% Tween™ 20 stock solution (IMV, 2jan.2019BM-1) and mixed by vortex to form an aqueous phase containing 0.5% Tween™ 20 by weight and 6.7 mg/mL anti-CTLA-4. The O/W emulsion was formed by filling a Normject syringe (Henke Sass Wolf, 18D30C8) with 0.7 mL of hydrophobic phase, filling another Normject syringe with 0.3 mL of the prepared aqueous phase, connecting each syringe using a Vyclic adapter (Vygon™, 210317FC), and passing the phase 120 times back and forth through the connector (passing the hydrophobic phase into the aqueous phase first). The final concentration of anti-CTLA-4 antibody in the emulsion was 2.0 mg/mL. The emulsion was confirmed to be O/W by water drop test, oil drop test, and cobalt strip test as shown below in Table 11. The final O/W emulsion formulation contained 2 mg/mL anti-CTLA-4, 66 mg/mL DOPC/Cholesterol, 0.1 mg/mL FP and 0.2 mg/mL dIdC.

TABLE 11

| Emulsion | | | Cobalt |
|---|---|---|---|
| Formulation | Drop Test (water) | Drop Test (oil) | Paper test |
| 6.7 mg/mL Anti- | Disperses (oil-in- | Sinks (oil-in-water) | Pink |
| CTLA4 and 0.5% | water) | | |
| Tween 20 in PBS and | | | |
| S•H₂O + | | | |
| DPX-FP in ISA51 | | | |
| Ratio: 0.3 mL: 0.7 mL | | | |

Formulations were prepared for treatment of the control groups. Mice in control groups 1, 2, 3, and 5 were treated with DPX-FP in ISA51 oil (i.e. hydrophobic phase only). Mice in control group 6 were treated with DPX-FP in ISA51 oil along with DPX-anti-CTLA-4 in ISA51 oil (containing anti-CTLA-4 antibody in a hydrophobic phase instead of peptide and adjuvant). Mice in group 7 were treated with DPX-FP/anti-CTLA-4 comprising anti-CTLA-4 antibody in addition to the peptides and adjuvant of DPX-FP. Mice in control groups 3, 4, and 5 received anti-CTLA-4 via i.p. injection. Mice in control group 9 received no treatment after tumor implantation. Mice in group 8 were treated with sub-cutaneous (s.c.) injections of the O/W emulsion according to the invention, with DPX-FP in the hydrophobic phase and the anti-CTLA-4 antibody in the aqueous phase. All treatments with DPX were by s.c. injection. The formulation of the treatments is shown in Table 12:

TABLE 12
Treatments

| | | | | O/W |
|---|---|---|---|---|
| | | | | emulsion |
| | | | | DPX-FP |
| | | | DPX- | (oil)/Anti- |
| | | DPX-Anti- | FP/Anti- | CTLA-4 |
| Formulation | DPX-FP | CTLA-4 | CTLA-4 | (water) |
| Antigens | FP (10 ug) | — | FP (10 ug) | FP (10 ug) |
| Drug | — | Anti-CTLA- | Anti-CTLA- | Anti-CTLA- |
| | | 4 (2 mg/ml) | 4 (2 mg/ml) | 4 (2 mg/ml) |
| Adjuvant | dIdC (20 ug) | — | dIdC (20 ug) | dIdC (20 ug) |
| Lipids | DOPC/chol | DOPC/chol | DOPC/chol | DOPC/chol |
| | (66 mg/mL) | (66 mg/mL) | (66 mg/mL) | (66 mg/mL) |
| Oil/Diluent | Montanide ™ | Montanide ™ | Montanide ™ | Montanide ™ |
| | ISA51 VG | ISA51 VG | ISA51 VG | ISA51 VG/ |
| | | | | PBS |
| Type | DPX | DPX | DPX | O/W |
| | | | | Emulsion |
| Groups | 1, 2, 3, 5, 6 | 6 | 7 | 8 |

Groups 2, 5, 6, 7, and 8 were also treated with mCPA as outlined in Table 13:

TABLE 13
Metronomic cyclophosphamide

| | Group 2, 5, 6, 7, 8 |
|---|---|
| Agent | CPA (Sigma) |
| Preparation | 0.133 mg/mL* |
| Dose | 20 mg/kg/day |
| Dose Volume | ~3 mL |
| Route | Drinking water (PO) |
| # of cycles | 2 |
| # of treatments per cycle | 7 × 24-hour daily periods (metronomic) |
| Location of treatment | Oral |
| Total Needed for Study | 20 aliquots |

The freeze-dried DPX-FP was prepared with the raw materials outlined in Table 14:

TABLE 14
DPX raw materials

| Material Type | Description | Source |
|---|---|---|
| Antigen | FP | NeoMPS |
| Drug | Anti-CTLA-4 mAb (Clone | BioXCell |
| | 9D9) | |
| Adjuvant | dIdC | Biospring |
| Lipids | DOPC/chol | Lipoid |
| Oil/Diluent | Montanide ™ ISA51 VG | SEPPIC |
| | Phosphate buffered saline | |
| | (PBS) | |

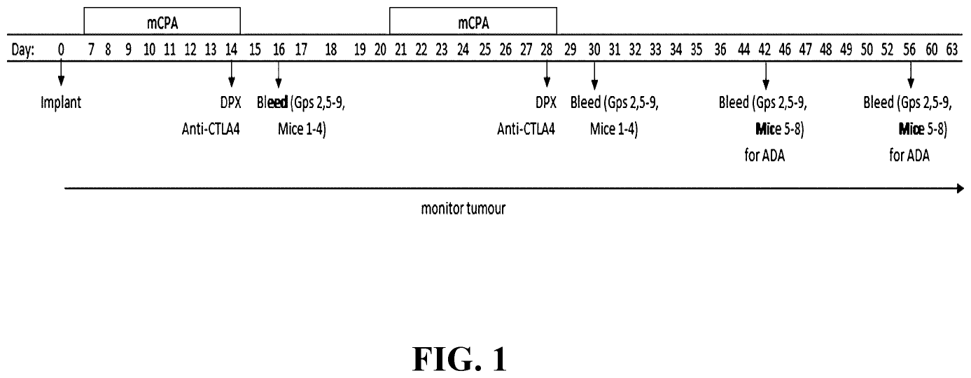

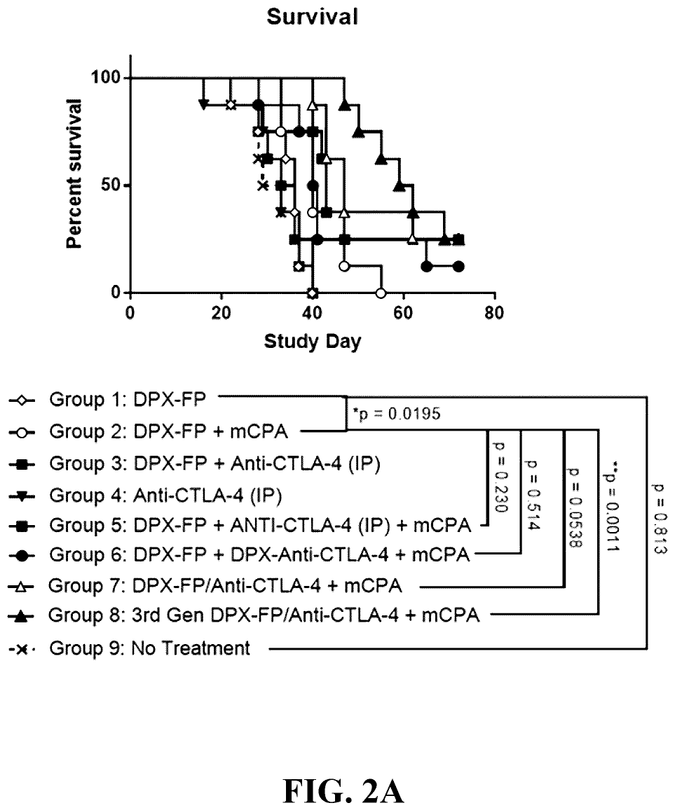

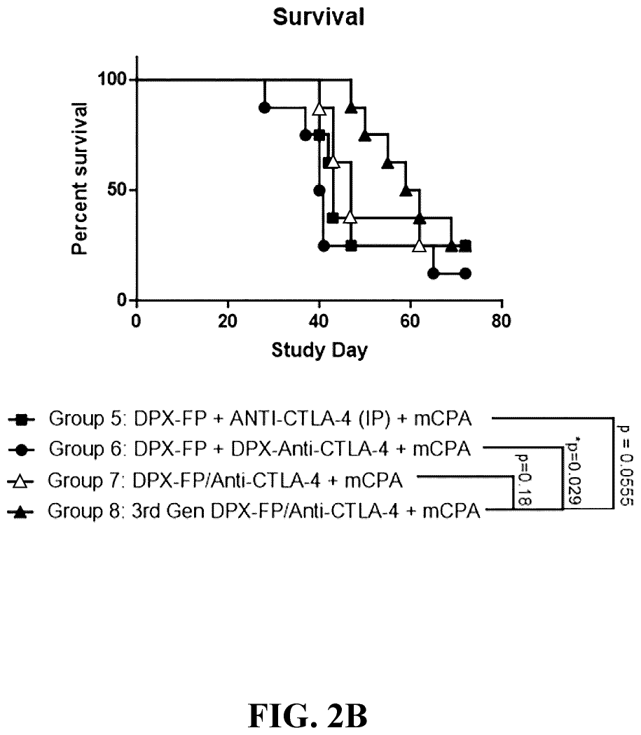

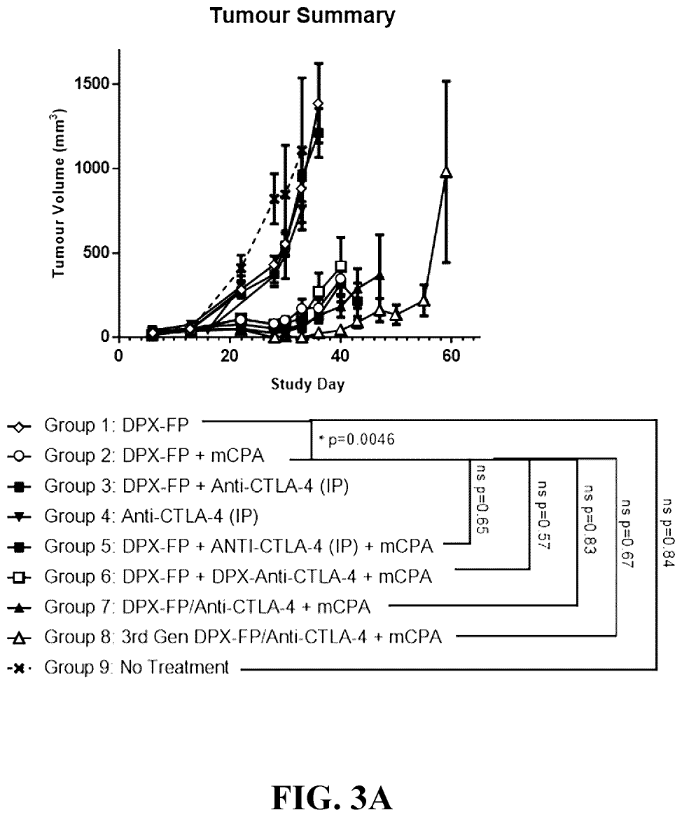

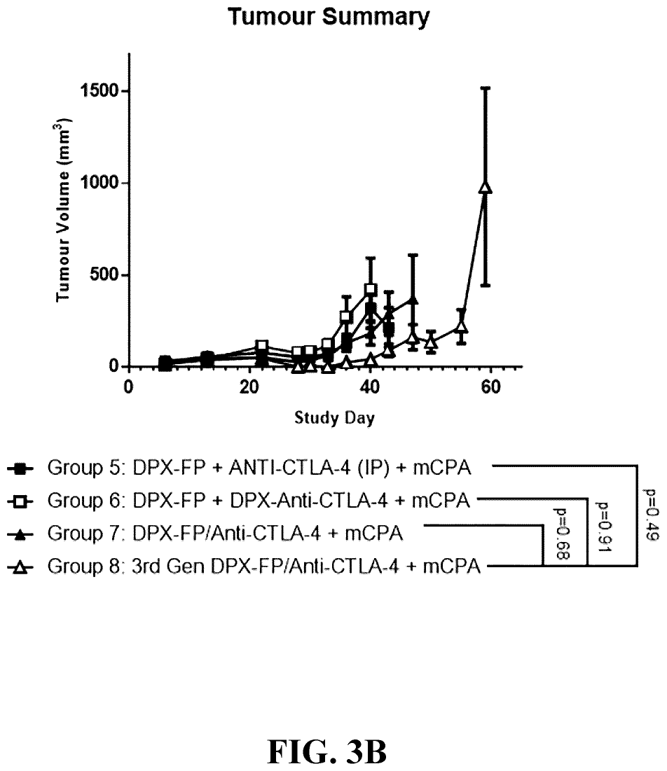

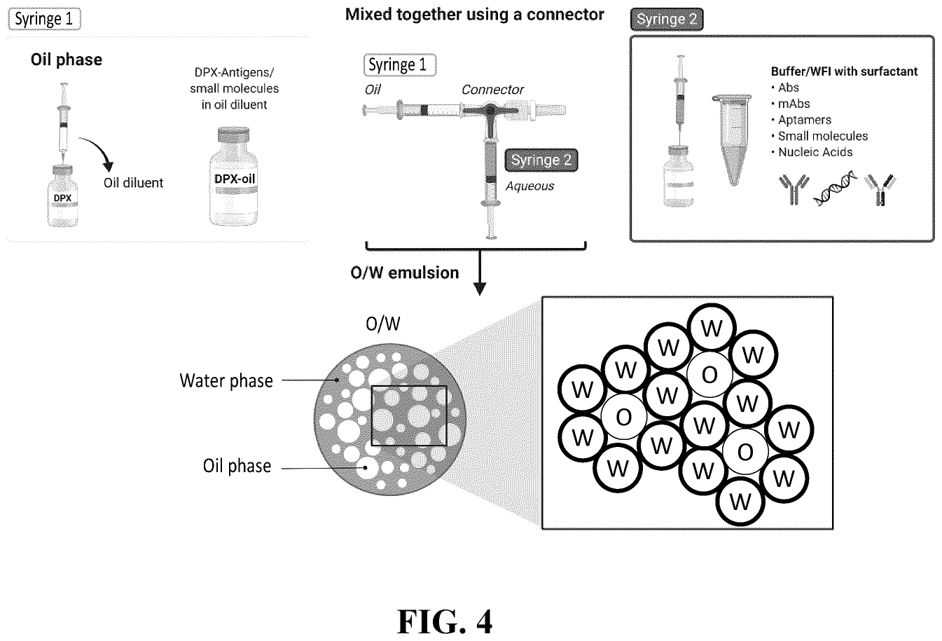

The study timeline is shown in FIG. 1. Mice (n=8 per group) were implanted with C3-10 cells at study day 0 (SD0) and treated with mCPA in their drinking water at 20 mg/kg/day for seven days starting at SD7 and SD21. Mice were either unadministered or administered with a combination of DPX-FP and/or anti-CTLA-4 (0.1 mg) by i.p. injection or by s.c. injection in DPX on SD14 and SD28. Results up to SD72 (FIG. 2A) show that mice treated DPX-FP+mCPA (Group 2) had a significant survival advantage over treatment with DPX-FP alone (Group 1; p=0.0195), and that anti-CTLA-4 delivered in an O/W emulsion according to the invention (Group 8) significantly improved survival compared to Group 2 (p=0.0011). There was a trend of increased survival in anti-CTLA-4 delivered by either i.p. injection (Group 5) or by s.c. injection in DPX (Groups 6, 7) compared to DPX-FP+mCPA alone (Group 2). Importantly, mice treated with the O/W emulsion formulation of the invention (group 8) had higher overall survival between SD48-SD60 than mice in control groups 5, 6, and 7 (FIG. 2B). Similarly, mice treated with the O/W emulsion formulation of the invention (group 8) had slower tumor growth compared to mice in control groups 5, 6, and 7 (FIGS. 3A and B). Survival statistical analysis was performed using the Mantel-Cox and Gehan-Breslow-Wilcoxon tests, ***p<0.001, *p<0.05. Tumor volume statistical analysis was performed by linear regression comparison, ***p<0.0001.

These results indicate that the DPX-FP in combination with anti-CTLA-4 delivered in an O/W emulsion formulation according to the invention improved survival and tumor control in treated mice compared to control mice receiving anti-CTLA-4 in hydrophobic DPX or by separate i.p. injection.

### Example 8

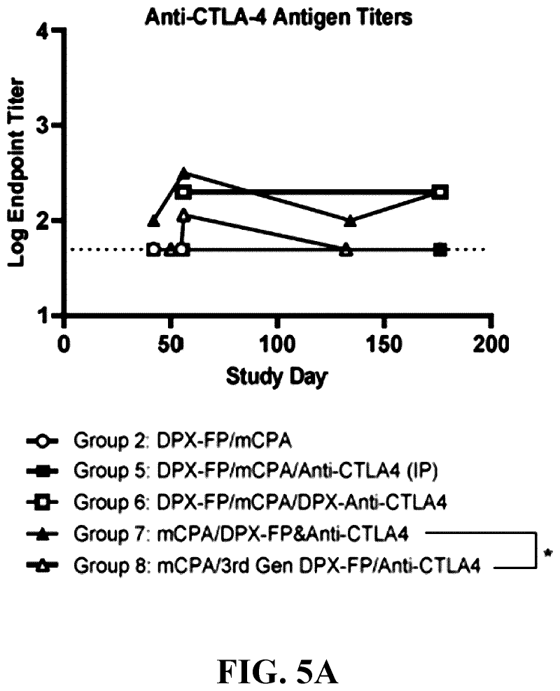

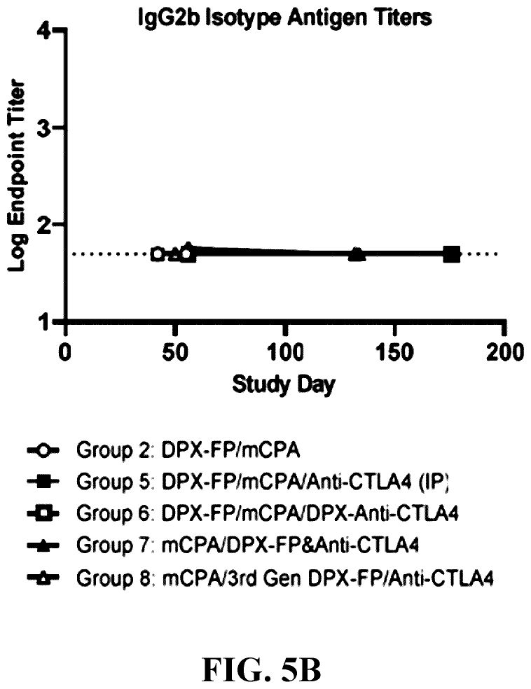

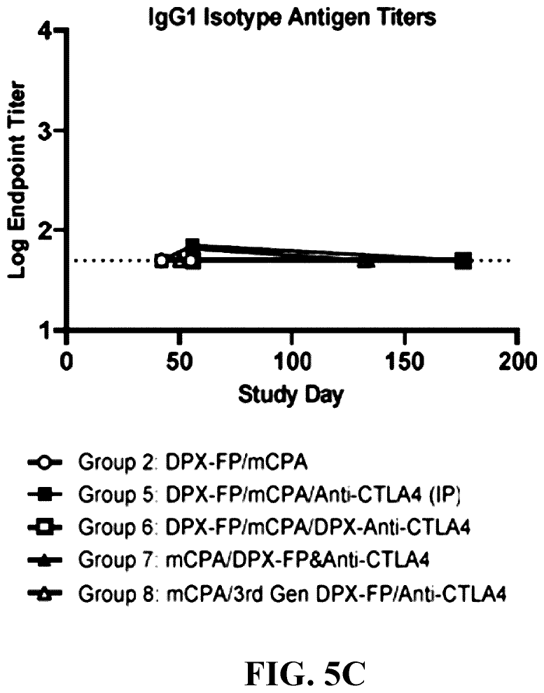

The mice from Example 7 were further tested for the formation of anti-drug antibody (ADA) against anti-CTLA4 antibody. Blood serum was collected for assessment of ADA formation on SD42 (Group 2, n=2, Group 5, n=2, Group 6, n=0, Group 7, n=3, Group 8, n=4) and SD55/56 (Group 2, n=1, Group 5, n=2, Group 6, n=2, Group 7, n=3, Group 8, n=6), and at the end of study (EOS) for mice that survived the initial tumour challenge and were re-challenged and not treated further (Group 2, n=0, Group 5, n=2, Group 6, n=1, Group 7, n=2, Group 8, n=2). ADA formation was detected by bridging ELISA with anti-CTLA-4 coating and detection antibody (FIG. 5A), IgG2b isotype control coating antibody and anti-CLA-4 detection antibody (FIG. 5B), and IgG1 isotype control coating antibody and anti-CTLA-4 detection antibody (FIG. 5C). Mice from Group 6, 7, and 8 developed ADA by SD55/56 which then either decreased (Groups 7 and 8) or remained constant (Group 6) to EOS. Statistical significance was assessed by one-way ANOVA using Tukey's multiple comparisons test, *p<0.05.

The results shown in FIG. 5 demonstrate that mice administered a composition of the present invention (Group 8) developed lower titers of unwanted ADA against the anti-CTLA4 antibody compared to control compositions (Groups 6 and 7). Indeed, when the anti-CTLA4 antibody was provided as an aqueous phase agent in a composition of the present invention (Group 8), significantly lower titers of ADA were generated compared to a control composition that did not comprise an O/W emulsion and in which the anti-CTLA4 antibody was provided in a hydrophobic carrier (Group 7).

### Example 9

Evaluation of the stability of the aqueous phase agent in the aqueous phase of the O/W emulsion.

O/W emulsions were prepared by combining the hydrophobic phase (DPX-empty in Montanide™ ISA 51 oil) and the aqueous phase (containing an aqueous phase agent) with 120 passes through a Vygon™ connector. Three exemplary O/W emulsions were prepared as shown in Table 15:

TABLE 15
Formulation Preparations

| Formulation 1 | Formulation 2 | Formulation 3 |
|---|---|---|
| DPX-Empty in oil + Oligonucleotide | DPX-Empty in oil + Cyclophosphamide | DPX-Empty in oil + anti-CTLA-4 |
| in aqueous O/W emulsion | in aqueous O/W emulsion | in aqueous O/W emulsion |
| CE20-191113-1 | CE20-200128-2 | CE20-200902-1 |
| 70% oil: Reconstitute 1 vial of | 70% oil: Reconstitute 1 vial of | 70% oil: Reconstitute 1 vial |
| DPX-Empty with 1 mL | DPX-empty with 1 mL | of DPX-Empty with 1 mL |
| Montanide ® ISA 51-remove | Montanide ® ISA 51 VG-remove | Montanide ® ISA 51 VG-remove |
| 700 μL and transfer to a 1-mL syringe | 700 μL and transfer to a 1-mL syringe | 700 μL and transfer to a 1-mL syringe |
| 30% aqueous with | 30% aqueous with | 30% aqueous with |
| 0.5% Tween 20: Sodium acetate, 0.1M, | 0.5% Tween 20: 1X PBS = 198.5 uL | 0.5% Tween 20: 4% Tween 20 = 37.5 uL |
| pH 7.0 = 242.5 uL 4% Tween 20 = 37.5 uL | 4% Tween 20 = 37.5 uL | anti-CTLA-4: |
| Oligonucleotide: | CPA: | |
| $0.2 mg / mL = \frac{0.2 mg / mL}{10 mg / mL} \times 1. mL = 20 µL$ | $8 mg / mL = \frac{8 mg / mL}{125 mg / mL} \times 1. mL = 64 µL$ | $2 mg / mL = \frac{2 mg / mL}{7.54 mg / mL} \times 1. mL = 265 µL$ |

As the aqueous phase agent, formulation 1 comprised oligonucleotide, formulation 2 comprised cyclophosphamide, and formulation 3 comprised anti-CTLA4 antibody. Montanide™ ISA 51 oil formed the hydrophobic phase of the formulations. To confirm that the formulations were O/W emulsions, the formulations were subjected to water drop tests, oil drop tests, and cobalt paper tests as described in Example 1. Both drop tests and the cobalt strip test were used for emulsion identification with oligonucleotide, cyclophosphamide and anti-CTLA4 formulations. All tests confirmed the formulations formed O/W emulsions.

For analysis by High Performance Liquid Chromatography (HPLC), the O/W emulsion formulations were centrifuged at 15,000 RPM for 30 minutes to separate oil (top) and aqueous (bottom) layers, and samples from both layers were analysed. Samples were prepared using n-Butanol Extraction method (for oil phase) and Total solubilization method (for aqueous phase). n-Butanol Extraction method (for oil phase): To 100 μL of the top layer oil sample 300 μL of 0.1M NaHCO₃ and 400 μL of water saturated 1-Butanol were added. Sample was vortexed to mix and centrifuged for 2 mins at 5000 RPM. The bottom layer was taken for analysis. Total solubilization method (for aqueous phase): To 75 μL of the bottom layer aqueous sample, up to 5 mL of mobile phase A was added. The mobile phases used for cyclophosphamide, oligonucleotide and anti-CTLA4 analysis were as follows, Cyclophosphamide: Mobile Phase A: 30% acetonitrile in water; Mobile Phase B: methanol. Oligonucleotide: Mobile Phase A: mix of tris, acetonitrile, water; Mobile Phase B: mix of tris, NaCL, acetonitrile, water. Anti-CTLA4: Mobile Phase A: 0.1% TFA/water; Mobile Phase B: 0.1% TFA/acetonitrile; Mobile Phase C: methanol. For the formulation containing cyclophosphamide aqueous phase agent, only the aqueous phase was tested as the HPLC method is not established to test the cyclophosphamide from oil phase.

HPLC analysis of the hydrophobic (oil) and aqueous (water) phases of formulation 1 (oligonucleotide aqueous phase agent) is shown in Table 16:

TABLE 16
Oligonucleotide HPLC Analysis

| | Polynucleotide Adjuvant | | |
|---|---|---|---|
| Acceptance Criteria (mg/mL) | | Amount | |
| Sample Assays (mg/mL) | Amount | sum | Recovery |
| Oligonucleotide control T = 0 | 0.3 | 0.3 | 75% |
| CE20-191113-1 T = 0 | 0 | 0.4 | 100% |
| Top Layer-Oil (sample prep 1) | | | |
| CE20-191113-1 T = 0 | 0.4 | | |
| Bottom Layer-Water | | | |
| (sample prep 1) | | | |
| CE20-191113-1 T = 0 | 0 | 0.44 | 110% |
| Top Layer-Oil (sample prep 2) | | | |
| CE20-191113-1 T = 0 | 0.44 | | |
| Bottom Layer-Water | | | |
| (sample prep 2) | | | |
| CE20-191113-1 T = 0 | 0 | 0.42 | 105% |
| Top Layer-Oil (sample prep 3) | | | |
| CE20-191113-1 T = 0 | 0.42 | | |
| Bottom Layer-Water | | | |
| (sample prep 3) | | | |
| CE20-191113-1 T = 2H | 0 | 0.32 | 80% |
| Top Layer-Oil | | | |
| CE20-191113-1 T = 2H | 0.32 | | |
| Bottom Layer-Water | | | |
| CE20-191113-1 T = 2H | 0 | 0.41 | 102.5% |
| Top Layer-Oil | | | |
| CE20-191113-1 T = 2H | 0.41 | | |
| Bottom Layer-Water | | | |

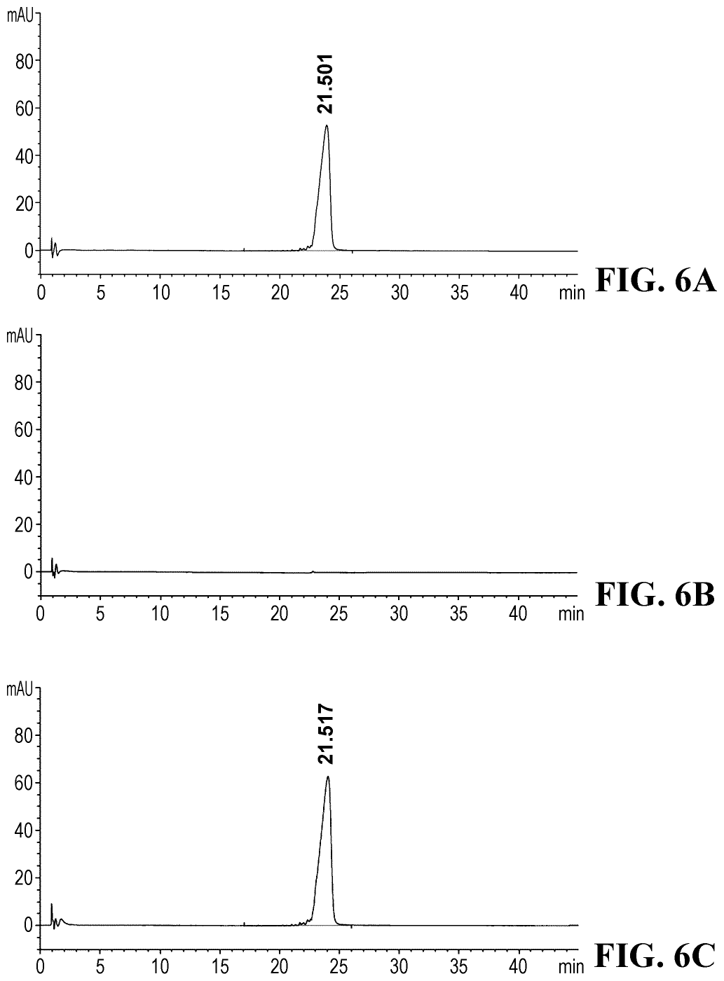

As seen in Table 16 the oligonucleotide aqueous phase agent remained in the aqueous phase (bottom layer-water) with 000 being found in the hydrophobic phase (top layer-oil) both immediately after formation of the O/W emulsion (T=0) and 2 hours after emulsification (T=2H). Each sample preparation was tested in duplicate. HPLC chromatograms of formulation 1 are shown in FIG. 6.

HPLC analysis of the aqueous (water) phase of formulation 2 (cyclophosphamide aqueous phase agent) is shown in Table 17:

TABLE 17
Cyclophosphamide HPLC Analysis

| | Cyclophosphamide | | |
|---|---|---|---|
| | Concentration | Target | % |
| ID | (mg/mL) | (mg/mL) | Recovery |
| Cyclophosphamide | 6.76 | 8.00 | 84.50 |
| control | | | |
| CE20-200128-2 | 7.34 | 8.00 | 90.00 |
| Bottom layer-Water | | | |

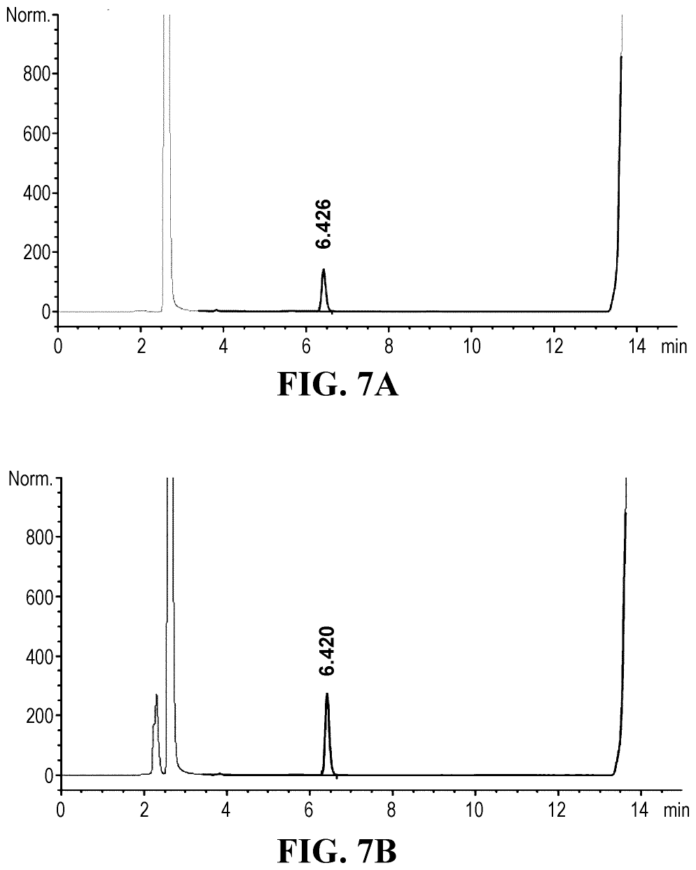

As seen in Table 17, the cyclophosphamide remained in the aqueous phase (bottom layer-water) of the sample. HPLC chromatograms of formulation 2 are shown in FIG. 7.

HPLC analysis of the hydrophobic (oil) and aqueous (water) phases of formulation 3 (anti-CTLA4 antibody aqueous phase agent) is shown in Table 18:

TABLE 18
Anti-CTLA4 HPLC Analysis

| | Anti-CTLA-4 | | |
|---|---|---|---|
| | Concentration | Target | |
| ID | (mg/mL) | (mg/mL) | % Recovery |
| CE20-200902-1 | 1.71 | 2.00 | 85.74 |
| Bottom layer- | | | |
| Water | | | |
| CE20-200902-1 | 0.00 | 0.00 | 0.00 |
| Top layer-oil | | | |

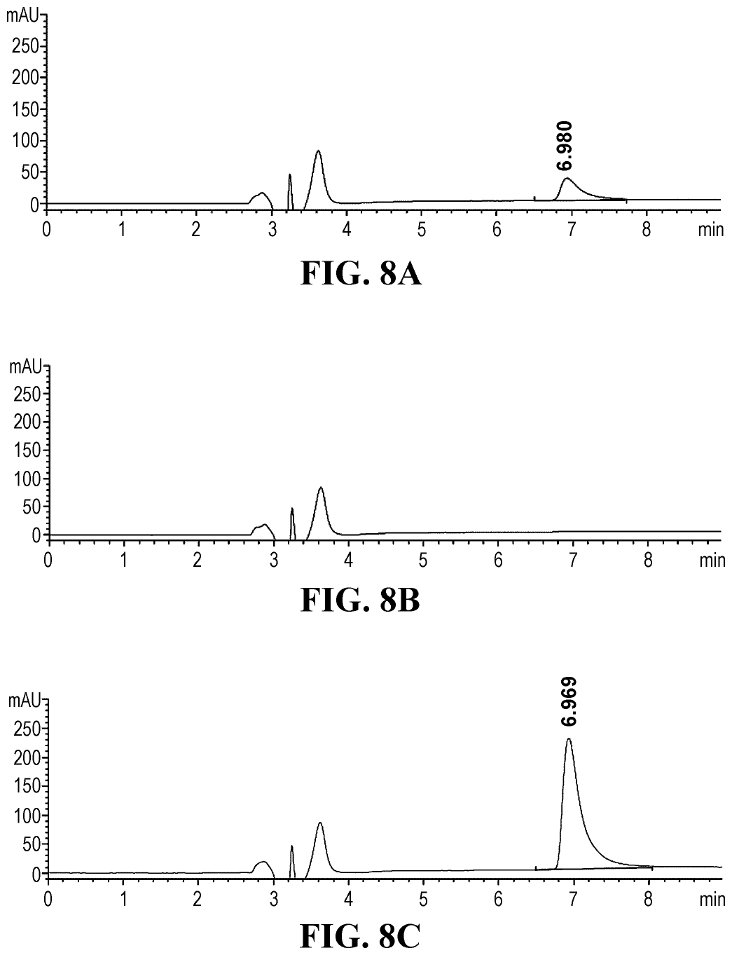

As seen in Table 18, the anti-CTLA-4 remained in the aqueous phase (bottom layer-water) of the sample. HPLC chromatograms of formulation 3 are shown in FIG. 8.

These results indicate that O/W formulations according to the present invention form emulsions in which the aqueous phase agent remains stably in the aqueous phase of the emulsion.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

### REFERENCES

1) Akbarzadeh, A.; Rezaei-Sadabady, R.; Davaran, S.; Joo, S. W.; Zarghami, N.; Hanifehpour, Y.; Samiei, M.; Kouhi, M.; and Nejati-Koshki, K. (2013) Liposome: classification, preparation, and applications, Nanoscale Res Lett. 8(1), 102.
2) Banga, A. K. Therapeutic Peptides and Proteins, Formulation, Processing and Delivery Systems (Lancaster, Pa.: Technomic Publishing Co., 1995).
3) Bertschinger, J.; Grabulovski, D.; and Neri, D. (2007) “Selection of single domain binding proteins by covalent DNA display”, Protein Eng Des Sel 20(2):57-68.
4) Beste, G.; Schmidt, F. S.; Stibora, T.; and Skerra, A. (1999) “Small antibody-like proteins with prescribed ligand specificities derived from the lipocalin fold”, Proc Natl Acad Sci USA. 96(5):1898-1903.
5) Bobst, A. M.; Langemeier, P. W.; Torrence, P. F.; De Clercq, E. (1981) Interferon Induction by Poly(inosinic acid) Poly(cytidylic acid) Segmented by Spin-Labels, Biochemistry 20(16), 4798-4803.
6) Boschek, C. B.; Apiyo, D. O.; Soares, T. A.; Engelmann, H. E.; Pefaur, N. B.; Straatsma, T. P.; and Baird, C. L. (2009) “Engineering an ultra-stable affinity reagent based on Top7”, Protein Engineering, Design and Selection 22(5):325-332.
7) Caruthers, M. H.; Beaucage, S. L.; Efcavitch, J. W.; Fisher, E. F.; Matteucci, M. D.; Stabinsky, Y. (1980) New chemical methods for synthesizing polynucleotides, Nucleic Acids Res. Symp. Ser. (7), 215-223.
8) Castle, J. C.: Kreiter, S.; Diekmann, J.; Lower, M.; van de Roemer, N.; de Graaf, J.; Selmi, A.; Diken, M.; Boegel, S.; Paret, C.; Koslowski, M.; Kuhn, A. N.; Britten, C. M.; Huber, C.; Turesi, O.; and Sahin, U. (2012) Exploiting the Mutanome for Tumor Vaccination. Cancer Res, 72(5): 1081-1091.
9) Celis, E.; Ou, D.; Otvos, L. Jr. (1988) Recognition of hepatitis B surface antigen by human T lymphocytes. Proliferative and cytotoxic responses to a major antigenic determinant defined by synthetic peptides, J Immunol. 140, 1808-1815.
10) Chong, P.; Zobrist, G.; Sia, C.; Loosmore, S.; Klein, M. (1992) Identification of T- and B-cell epitopes of the S2 and S3 subunits of pertussis toxin by use of synthetic peptides, Infect Immun. 60, 4640-4647.
11) Collins, P. L.; Olmsted, R. A.; Johnson, P. R. (1990) The small hydrophobic protein of human respiratory syncytial virus: comparison between antigenic subgroups A and B, J Gen Virol. 71(7), 1571-6.
12) Demotz, S.; Lanzavecchia, A.; Eisel, U.; Niemann, H.; Widmann, C.; Corradin, G. P. (1989) Delineation of several DR-restricted epitopes in tetanus toxin, J Immunol. 142, 394-402.
13) Desmet, J.; Verstraete, K.; Bloch, Y.; Lorent, E.; Wen, Y.; Devreese, B.; Vandenbroucke, K.; Loverix, S.; Hettmann, T.; Deroo, S.; Somers, K.; Henderikx, P.; Lasters, I.; and Savvides, S. N. (2014). “Structural basis of IL-23 antagonism by an Alphabody protein scaffold”, Nature Communications 5:5237.
14) Diethelm-Okita, B. M.; Okita, D. K.; Banaszak, L.; Conti-Fine, B. M. (2000) Universal epitopes for human CD4+ cells on tetanus and diphtheria toxins, J. Infect. Dis. 181, 1001-1009.
15) Feldwisch, J.; and Tolmachev, V. (2012) “Engineering of affibody molecules for therapy and diagnostics”, Methods Mol Biol 899:103-26.
16) Flynn, J. L. (1994) Recombinant BCG as an antigen delivery system, Cell. Mol. Biol. 40 (suppl. 1), 31-36.
17) Frezard, F. (1999) Liposomes: from biophysics to the design of peptide vaccines, Braz. J. Med. Bio. Res. 32, 181-189.
18) Gebauer, M.; and Skerra, A. (2009) “Engineered protein scaffolds as next-generation antibody therapeutics”, Curr Opinion in Chemical Biology 13:245-255.
19) Grabulovski, D.; Kaspar, M.; and Neri, D. (2007) “A Novel, Non-immunogenic Fyn SH3-derived Binding Protein with Tumor Vascular Targeting Properties”, J Biol Chem 282:3196-3204.
20) Green, L. L.; Hardy, M. C.; Maynard-Currie, C. E.; Tsuda, H.; Louie, D. M.; Mendez, M. J.; Abderrahim, H.; Noguchi, M.; Smith, D. H.; Zeng, Y.; David, N. E.; Sasai, H.; Garza, D.; Brenner, D. G.; Hales, J. F.; McGuinness, R. P.; Capon, D. J.; Klapholz, S.; Jakobovits, A. (1994) “Antigen-specific human monoclonal antibodies from mice engineered with human Ig heavy and light chain YACs”, Nature Genet 7:13-21.
21) Greene, J. J.; Alderfer, J. L.; Tazawa, I.; Tazawa, S.; Ts'o, P. O.; O'Malley, J. A.; Carter, W. A. (1978) Interferon induction and its dependence on the primary and secondary structure of poly(inosinic acid).poly(cytidylic acid), Biochemistry 17(20), 4214-4220.
22) Greenfield, E. A. (ed.) (2014) “Antibodies: A Laboratory Manual”, Cold Spring Harbor Laboratory Press, 2^{nd} Edition.
23) Gregoriadis G. (1990) Immunological adjuvants: a role for liposomes, Immunol. Today 11, 89-97.
24) Hagstrom, N. J.; Couto, L. B.; Scallan, C.; Burton, M.; McCleland, M. L.; Fields, P. A.; Arruda, V. R.; Herzog, R. W. and High, K. A. (2000) Blood, 95, 2536-2542.
25) Hendrix, C. W.; Margolick, J. B.; Petty, B. G.; Markham, R. B.; Nerhood, L.; Farzadegan, H.; Ts'o, P. O.; Lietman, P. S. (1993) Biologic effects after a single dose of poly(I):poly(C12U) in healthy volunteers, Antimicrob. Agents Chemother. 37(3), 429-435.
26) High, N.; Mounier, J.; Prévost, M. C.; Sansonetti, P. J. (1992) IpaB of Shigella flexneri causes entry into epithelial cells and escape from the phagocytic vacuole, EMBO J 11, 1991-1999.
27) Hollinger, P.; and Hudson, P. J. (2005) “Engineered antibody fragments and the rise of single domains”, Nat Biotechnol 23(9):1126-1136.
28) Horn, T.; Vasser, M. P.; Struble, M. E.; Crea, R. (1980) Synthesis of oligonucleotides on cellulose. Part II: Design and synthetic strategy to the synthesis of 22 oligodeoxynucleotides coding for gastric inhibitory polypeptide (GIP), Nucleic Acids Symp. Ser. (7), 225-232.
29) Johnson, K. S.; and Chiswell, D. J. (1993) “Human antibody engineering”, Current Opinion in Structural Biology 3:564-571.
30) Kawaoka, Y.; Yamnikova, S.; Chambers, T. M.; Lvov, D. K.; Webster, R. G. (1990) Molecular characterization of a new hemagglutinin, subtype H14, of influenza A virus, Virology 179(2), 759-767.
31) Kieny, M. P.; Lathe, R.; Drillien, R.; Spehner, D.; Skory, S.; Schmitt, D.; Wiktor, T.; Koprowski, H. and Lecocq, J. P. (1984) Expression of rabies virus glycoprotein from a recombinant vaccinia virus, Nature 312, 163-166.
32) Li, Z. L. and Paulin, D. (1991) High level desmin expression depends on a muscle-specific enhancer, J. Biol. Chem. 266, 6562-6570.
33) Li, Z. L. and Paulin, D. (1993) Different factors interact with myoblast-specific and myotube-specific enhancer regions of the human desmin gene, J. Biol. Chem. 268, 10403-10415.
34) Li, X.; Eastman, E. M.; Schwartz, R. J.; and Draghia-Akli, R. (1999) Synthetic muscle promoters: activities exceeding naturally occurring regulatory sequences, Nat. Biotechnol 17, 241-245.
35) Lonberg, N.; Taylor, L. D.; Harding, F. A.; Trounstine, M.; Higgins, K. M.; Schramm, S. R.; Kuo, C.-C.; Mashayekh, R.; Wymore, K.; McCabe, J. G.; O'Regan, D. M.; O'Donnell, S. L.; Lapachet, E. S. G.; Bengoechea, T.; Fishwild, D. M.; Carmack, C. E.; Kay, R. M.; and Huszar, D. (1994) “Antigen-specific human antibodies from mice comprising four distinct genetic modifications”, Nature 368:856-859.
36) McCafferty, J.; Griffiths, A. D.; Winter, G.; and Chiswell, D. J. (1990) “Phage antibodies: filamentous phage displaying antibody variable domains”, Nature 348:552-553.
37) Medaglini, D.; Pozzi, G.; King, T. P. and Fischetti, V. A. (1995) Mucosal and systemic immune responses to a recombinant protein expressed on the surface of the oral commensal bacterium Streptococcus gordonii after oral colonization, Proc. Natl. Acad. Sci. USA 92, 6868-6872.
38) Mekalanos, J. J.; Swartz, D. J.; Pearson, G. D. N.; Harford, N.; Groyne, F. and de wilde, M. (1983) Cholera toxin genes: nucleotide sequence, deletion analysis and vaccine development, Nature 306, 551-557.
39) Merrifield, B. (1997) Concept and early development of solid-phase peptide synthesis, Methods Enzymol. 289, 3-13.
40) Mouratou, B.; Behar, G.; Paillard-Laurance, L.; Colinet, S.; and Pecorari, F. (2012) “Ribosome display for the selection of Sac7d scaffolds”, Methods Mol Biol 805:315-31.
41) Moyle, P. M.; Toth. I. (2008) Self-adjuvanting lipopeptide vaccines, Curr. Med. Chem.
   15(5), 506-516.
42) Nakayama, K.; Kelly, S. M. and Curtis, R. III (1988) Construction of an Asd+ expression cloning vector: stable maintenance and high level expression of cloned genes in a Salmonella strain, Bio/Technology 6, 693-697.
43) van der Neut Kolfschoten, M.; Schuurman, J.; Losen. M.; Bleeker, W. K.; Martinez-Martinez, P.; Vermeulen, E.; den Bleker, T. H.; Wiegman, L.; Vink, T.; Aarden, L. A.; De Baets, M. H.; van de Winkel, J. G.; Aalberse, R. C.; Parren, P. W. (2007) “Anti-inflammatory activity of human IgG4 antibodies by dynamic Fab arm exchange”, Science 317(5844):1554-1557.
44) Pardoll, D. M. (2012) “The blockade of immune checkpoints in cancer immunotherapy”, Nature Reviews Cancer 12:252-264.
45) Roberge, J. Y.; Beebe, X.; Danishefsky, S. J. (1995) A strategy for a convergent synthesis of N-linked glycopeptides on a solid support, Science 269(5221), 202-204.
46) Schlatter, D.; Brack, S.; Banner, D. W.; Batey, S.; Benz, J.; Bertschinger, J.; Huber, W.; Joseph, C.; Rufer, A. C.; van der Klooster, A.; Weber, M.; Grabulovski, D.; and Hennig, D. (2012) “Generation, characterization and structural data of chymase binding proteins based on the human Fyn kinase SH3 domain”, MAbs 4(4):497-508.
47) Sizemore, D. R.; Branstrom, A. A.; and Sadoff, J. C. (1995) Attenuated Shigella as a DNA Delivery Vehicle for DNA-Mediated Immunization, Science 270, 299-303.
48) Slingluff, C. L. Jr.; Yamshchikov, G.; Neese, P.; Galavotti, H.; Eastham, S.; Engelhard, V. H.; Kittlesen, D.; Deacon, D.; Hibbitts, S.; Grosh, W. W.; Petroni, G.; Cohen, R.; Wiemasz, C.; Patterson, J. W.; Conway, B. P.; Ross, W. G. (2001) Phase I trial of a melanoma vaccine with gp100(280-288) peptide and tetanus helper peptide in adjuvant: Immunologic and clinical outcomes, Clin Cancer Res. 7, 3012-3024.
49) So, N. S. Y.; Ostrowski, M. A.; Gray-Owen, S. D. (2012) Vigorous Response of Human Innate Functioning IgM Memory B Cells upon Infection by Neisseria gonorrhoeae, J. Immunol. 188(8), 4008-4022.
50) Srivastava, P. (2015) Neoepitopes of Cancers: Looking Back, Looking Ahead, Cancer Immunol. Res. 3, 969-977.
51) Suderman, R. J., Rice, D. A., Gibson, S. D., Strick, E. J., Chao, D. M. (2017) “Development of polyol-responsive antibody mimetics for single-step protein purification”, Protein Expression and Purification 134:114-124.
52) Tartaglia, J.; Perkus, M. E.; Taylor, J.; Norton, E. K.; Audonnet, J. C.; Cox, W. I.; Davis, S. W.; van der Hoeven, J.; Meignier, B.; Riviere, M.; et al. (1992) NYVAC: a highly attenuated strain of vaccinia virus, Virology 188, 217-232.
53) Taylor, L. D.; Carmack, C. E.; Huszar, D.; Higgins, K. M.; Mashayekh, R.; Sequar, G.; Schramm, S. R.; Kuo, C. C.; O'Donnell, S. L.; Kay, R. M.; et al. (1994) “Human immunoglobulin transgenes undergo rearrangement, somatic mutation and class switching in mice that lack endogenous IgM”, Int. Immun. 6:579-591.
54) Taylor, J.; Meignier, B.; Tartaglia, J.; Languet, B.; VanderHoeven, J.; Franchini, G.; Trimarchi, C.; and Paoletti E. (1995) Biological and immunogenic properties of a canarypox-rabies recombinant, ALVAC-RG (vCP65) in non-avian species, Vaccine 13, 539-549.
55) van Doom, E.; Liu, H.; Huckriede, A.; Hak, E. (2016) Safety and tolerability evaluation of the use of Montanide ISA™ 51 as vaccine adjuvant: A systematic review, Human Vaccines and Immunotherapeutics 12(1):159-169.
56) Webster, R. G.; Laver, W. G.; Air, G. M. Antigenic variation among type A influenza viruses, p. 127-168. In: Palese, P. & Kingsbury, D. W., eds. Genetics of influenza viruses. (New York: Springer-Verlag, 1983).
57) Weidle, U. H.; Auer, J.; Brinkmann, U.; Georges, G.; Tiefenthaler, G. (2013) “The emerging role of new protein scaffold-based agents for treatment of cancer” Cancer Genomics Proteomics 10(4):155-68.
58) Li, Z. L.; Lilienbaum, A.; Butler-Browne, G.; Paulin, D. (1989) Human desmin-coding gene: complete nucleotide sequence, characterization and regulation of expression during myogenesis and development, Gene 78, 243-254.

What is claimed:

1. A composition for delivering at least two agents to a subject comprising:
i) a hydrophobic phase; and
ii) an aqueous phase;
wherein the composition is an emulsion of the hydrophobic phase in the aqueous phase, wherein the hydrophobic phase comprises at least one hydrophobic phase agent, and wherein the aqueous phase comprises at least one aqueous phase agent.

2. The composition of claim 1, wherein the ratio of the hydrophobic phase to the aqueous phase is between 70:30 v/v to 50:50 v/v.

3. The composition of claim 1 or 2, wherein the hydrophobic phase comprises one or more hydrophobic substances selected from vegetable oil, nut oil, mineral oil, mannide oleate in mineral oil, and sorbitan monooleate in mineral oil.

4. The composition of claim 3, wherein the hydrophobic phase comprises a dried preparation of the at least one hydrophobic phase agent reconstituted in the hydrophobic substance.

5. The composition of claim 1 or 2, wherein the hydrophobic phase comprises a phospholipid and cholesterol in a hydrophobic substance selected from vegetable oil, nut oil, mineral oil, mannide oleate in mineral oil, and sorbitan monooleate in mineral oil.

6. The composition of claim 5, wherein the phospholipid is DOPC.

7. The composition of claim 5 or 6, wherein the hydrophobic phase comprises a dried composition of DOPC, cholesterol, and the at least one hydrophobic phase agent reconstituted in mannide oleate in mineral oil or sorbitan monooleate in mineral oil.

8. The composition of any one of claims 1 to 7, wherein the at least one hydrophobic phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.

9. The composition of claim 8, wherein the at least one hydrophobic phase agent is an antigen and an adjuvant.

10. The composition of claim 8, wherein the at least one hydrophobic phase agent is an antigen, a T-helper epitope, and an adjuvant.

11. The composition of any one of claims 1 to 10, wherein the aqueous phase comprises water, an aqueous solution, or a combination thereof.

12. The composition of claim 11, wherein the aqueous phase further comprises an emulsifier.

13. The composition of claim 12, wherein the emulsifier is polysorbate 20, polysorbate 80, sorbitan monolaurate, or sorbitan monooleate.

14. The composition of any one of claims 11 to 13, wherein the aqueous phase comprises a dried preparation of the at least one aqueous phase agent reconstituted in water, an aqueous solution, or a combination thereof.

15. The composition of any one of claims 1 to 14, wherein the at least one aqueous phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.

16. The composition of claim 15, wherein the at one aqueous phase agent is an antibody, a functional equivalent of an antibody, a functional fragment of an antibody, or an antibody mimetic that binds to CTLA-4.

17. A method for making a composition for delivering at least two agents to a subject, said method comprising:
i) providing a hydrophobic phase comprising at least one hydrophobic phase agent;
ii) providing an aqueous phase comprising at least one aqueous phase agent;
iii) mixing the hydrophobic phase and the aqueous phase to produce an emulsion of the hydrophobic phase in the aqueous phase.

18. The method of claim 17, wherein the ratio of the hydrophobic phase to the aqueous phase is between 70:30 v/v to 50:50 v/v.

19. The method of claim 17 or 18, wherein the hydrophobic phase comprises one or more hydrophobic substances selected from vegetable oil, nut oil, mineral oil, mannide oleate in mineral oil, and sorbitan monooleate in mineral oil.

20. The method of claim 19, wherein the hydrophobic phase is produced by reconstituting a dried preparation of the at least one hydrophobic phase agent in the hydrophobic substance.

21. The method of claim 17 or 18, wherein the hydrophobic phase comprises a phospholipid and cholesterol in a hydrophobic substance selected from vegetable oil, nut oil, mineral oil, mannide oleate in mineral oil, and sorbitan monooleate in mineral oil.

22. The method of claim 21, wherein the phospholipid is DOPC.

23. The method of claim 21 or 22, wherein the hydrophobic phase is produced by reconstituting a dried composition of DOPC, cholesterol, and the at least one hydrophobic phase agent in mannide oleate in mineral oil or sorbitan monooleate in mineral oil.

24. The method of any one of claims 17 to 23, wherein the at least one hydrophobic phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.

25. The method of any one of claims 17 to 24, wherein the aqueous phase comprises water, an aqueous solution, or a combination thereof.

26. The method of claim 25, wherein the aqueous phase further comprises an emulsifier.

27. The method of claim 26, wherein the emulsifier is polysorbate 20, polysorbate 80, sorbitan monolaurate, or sorbitan monooleate.

28. The method of any one of claims 25 to 27, wherein the aqueous phase is produced by reconstituting a dried preparation of the at least one aqueous phase agent in water, an aqueous solution, or a combination thereof.

29. The method of any one of claims 17 to 28, wherein the at least one aqueous phase agent is a small molecule drug, an antibody, a functional fragment of an antibody, a functional equivalent of an antibody, an antibody mimetic, an immunomodulatory agent, an antigen, a T-helper epitope, an adjuvant, an allergen, a DNA polynucleotide, or an RNA polynucleotide.

30. A composition produced by the method of any one of claims 17 to 29.

31. A method for delivering at least two agents to a subject, said method comprising administering to the subject the composition of any one of claims 1 to 16.

32. A kit comprising:
a) a first container comprising a dried preparation of at least one hydrophobic phase agent;
b) a second container comprising one or more hydrophobic substances; and
c) a third container comprising an aqueous solution comprising at least one aqueous phase agent.

33. A kit comprising:
a) a first container comprising a dried preparation of at least one hydrophobic phase agent;
b) a second container comprising one or more hydrophobic substances;
c) a third container comprising a dried preparation of at least one aqueous phase agent; and
d) a fourth container comprising water, an aqueous solution, or a combination thereof.
